(19) United States
(12) Patent Application Publication
(10) Pub. No.: US 2012/0277180 A1
(43) Pub. Date: Nov. 1, 2012

(54) Cofactors and Methods for Use for Individuals

(75) Inventors: Nicholas Marini, Greenbrae, CA (US); Jasper Rine, Point Richmond, CA (US); Dennis Austin Gilbert, San Francisco, CA (US); Bruce Cohen, Los Altos, CA (US)

(73) Assignees: VITAPATH GENETICS, INC, Foster City, CA (US); THE REGENTS OF THE UNIVERSITY OF CALIFORNIA, Oakland, CA (US)

(21) Appl. No.: 13/499,391

(22) PCT Filed: Sep. 30, 2010
(86) PCT No.: PCT/US10/51006

§ 371 Date: Jul. 12, 2012

Related U.S. Application Data
(60) Provisional application No. 61/247,485, filed on Sep. 30, 2009.

(51) Int. Cl. A61K 31/519; C40B 40/06; C12Q 1/68; C40B 30/04; C07C 403/08; C07C 50/14; C07C 401/00; C07D 307/62; A61K 31/07; A61K 31/122; A61K 31/59; A61K 31/375; C07D 311/72; A61K 31/355; C07D 415/00; A61K 31/51; A61K 31/525; C07D 487/04; A61K 31/455; C07D 213/80; A61K 31/4415; C07D 213/67; C07D 475/04; A61K 31/714; C07H 23/00; C07D 495/04; A61K 31/4188; C07C 235/12; A61K 31/197; C07C 215/40; A61K 31/14; A61P 25/00; A61P 43/00; A61P 15/00; G09B 19/00; C07H 21/00

(52) U.S. Cl. 514/52; 536/23.2; 506/16; 435/6.11; 506/9; 568/824; 552/299; 552/653; 549/315; 514/725; 514/681; 514/168; 514/167; 514/474; 549/408; 514/458; 544/327; 514/276; 514/251; 544/251; 514/356; 546/318; 514/345; 546/301; 514/249; 544/261; 536/26.4; 548/303.7; 514/387; 562/569; 514/563; 564/293; 514/642; 434/127

(57) ABSTRACT

Provided herein are methods and systems for identifying one or more cofactors such as vitamins for individuals based on the genetic makeup of the individual by detecting the presence or absence of at least one genetic variant, determining a predisposition to cofactor remediable condition, generating a personalized nutritional advice plan based on the genetic variant. Also provided herein are formulations of cofactors determined by the genetic make-up of the individual and methods of determining and producing these formulations.

### BACKGROUND

The folate/homocysteine metabolic pathway constitutes a network of enzymes and enzymatic pathways that metabolize folate and/or affect homocysteine. The pathways are linked via the methionine synthase reaction, and marginal folate deficiencies in cell cultures, animal model systems and in humans impair homocysteine remethylation (see, for example, Stover P J. 2004. Physiology of folate and vitamin B₁₂ in health and disease. Nutr Rev 62:S3-12).

Folate inadequacy has been linked to neural tube defects (“NTDs”) as well as other birth defects and adverse pregnancy outcomes, such as orofacial clefts, pre-eclampsia, pre-term delivery/low birth weight, and recurrent early spontaneous abortion (see, for example, Mills et al., 1995. Homocysteine metabolism in pregnancies complicated by neural tube defects. Lancet 345:149-1151), Folate inadequacy has also been associated with cardiovascular disease, coronary artery disease, ischemic stroke, atherosclerosis, thrombosis, retinal artery occlusion, Down's Syndrome, colorectal cancer, breast cancer, lung cancer, prostate cancer, depression, schizophrenia, Alzheimer's Disease/Dementia, age-related macular degeneration, and glaucoma.

All the metabolic steps in the folate/homocysteine metabolic pathway are potentially relevant to conditions and diseases associated with folate inadequacy and/or homocysteine metabolism. Enzymes involved in folate/homocysteine metabolism that are implicated include, e.g., bifunctional enzyme AICAR Transformylase and IMP Cyclohydrolase (ATIC), glycinamide ribonucleotide transformylase (GART), methionine adenosyltransferase I, alpha (MAT1A), methionine adenosyltransferase II, alpha (MAT2A), methylenetetrahydrofolate reductase (MTHFR), and methenyltetrahydrofolate synthetase (MTHFS). Folate inadequacy also impairs methylation mediated by S-adenosyl-methionine (“SAM”), which is an allosteric inhibitor of both MTHFR and CBS (see, for example, Kraus et al, 1999, Cystathionine 3-synthase mutations in homocystinuria. Hum Mut 13:362-375; Daubner et al, 1982. In Flavins and Flavoproteins, eds. Massey, V. & Williams, C. H, (Elsevier, New York), pp. 165-172). Elevations in the S-adenosyl-homocysteine:S-adenosylmethionine (SAH/SAM) ratios have been proposed in the mechanism of NTD development.

5,10-Methylenetetrahydrofolate reductase (MTHFR) is involved in the folate-dependent multistep pathway in which homocysteine is converted to methionine. Decreased conversion of homocysteine can lead to hyperhomocysteinemia.

Several rare mutations of MTHFR have been identified that are associated with clinical MTHFR deficiency, an autosomal recessive disorder. The clinical symptoms of MTHFR deficiency are highly variable and include developmental delay, motor and gait abnormalities, seizures, and premature vascular disease.

Common polymorphisms of MTHFR have also been described, including the functionally impaired allele A222V. The genetic association of common polymorphisms with disease has not been consistent. This may be due in part to compensatory effects of folate availability that mask an underlying risk of disease, as well as the contribution of as yet unidentified low frequency impaired alleles to such diseases. Interestingly, common polymorphisms have been associated with individual variation in the efficacy and toxicity of chemotherapeutics, such as methotrexate and 5-fluorouracil.

An assay for functional complementation of the yeast gene met11 has been described (Shan et al., JBC, 274:3261 3-32618, 1999). In this assay, wildtype human MTHFR was shown to complement a met11 mutation in S. cerevisiae. However, this assay was not sensitive to quantitative changes in activity due to MTHFR mutations, as demonstrated by the similar ability of the functionally impaired allele A222V to complement the yeast mutation as compared to the wild-type enzyme; nor was this assay sensitive to the effects of folate availability.

In addition to folate utilizing enzymes, a handful of vitamin B₆- and B₁₂-dependent enzymes and enzymatic pathways are relevant to homocysteine metabolism, NTDs and other birth defects and adverse pregnancy outcomes. For example, defects in the B₆ utilizing enzyme cystathionine-13-synthase (“CBS”) lead to accumulation of homocysteine (Kraus et al, 1999. Cystathionine 13-synthase mutations in homocystinuria. Hum Mut 13:362-375). As well, single nucleotide polymorphisms (“SNPs”) of the B₆ utilizing enzyme cystathionine-γ-lyase (“CTH”) have also been associated with homocysteinemia (Wang et al., 2004. Single nucleotide polymorphism in CTH associated with variation in plasma homocysteine concentration. Clin Genet 65:483-486).

### SUMMARY

The invention derives in part from the development of novel in vivo assays for identifying impaired alleles of enzyme-encoding genes within metabolic pathways and determining their sensitivity to cofactor remediation. Compound yeast mutants, comprising a first mutation allowing for complementation by a functionally homologous enzyme of interest, and a second mutation (or group of mutations) rendering the strain dependent upon supplementation with a cofactor, provide for the study of enzyme complementation as a function of cofactor availability. Cofactor-sensitive impaired alleles, including remediable alleles, may be identified and the cofactor-availability:enzyme-activity relationship may be analyzed using assays disclosed herein, The results obtained may be used to inform prophylactic and therapeutic nutrient supplementation approaches to prevent and treat conditions and diseases associated with metabolic enzyme dysfunction and aberrant metabolism.

The present invention also derives in part from the demonstration for the first time herein that cofactor remediation of low-frequency impaired alleles in enzyme-encoding genes is surprisingly common. As exemplified herein, multiple cofactor-sensitive genes in a metabolic pathway can each have multiple low frequency mutations in the population. Taken together, these mutations collectively have a more significant impact on the metabolic pathway than would be apparent from examination of a single low frequency impaired allele of a single gene. Moreover, since cells heterozygous for a plurality of such low frequency impaired alleles display quantitative defects, the aggregate frequencies of such individually rare alleles may contribute to common phenotypes even in the absence of more common polymorphism(s). Such low-frequency impaired alleles having impact on the pathway may also contribute to the phenotypic variation that is observed with common polymorphisms. Accordingly, the present invention contemplates diagnostic and prognostic methods focused in particular on the detection and characterization of such low frequency impaired alleles in enzyme-encoding genes, and determination of their effective remediation.

The present invention also derives in part from the specific application of these assays to identify and characterize novel low frequency impaired alleles in enzyme-encoding genes involved in folate/homocysteine metabolism in particular. As demonstrated herein with respect to MTHFR, a number of low-frequency impaired alleles exist that can cumulatively contribute to enzyme deficiency but can also be resolved by cofactor supplementation. The invention also derives in part from the finding that impaired alleles of MTHFR comprise sequence changes that map to the coding sequence of the N-terminal catalytic domain of the enzyme.

In one aspect, the invention therefore provides in vivo assays for detecting impaired but remediable alleles of enzyme-encoding genes involved in folate/homocysteine metabolism including, e.g., ATIC, GART, MAT1A, MAT2A, MTHFR, and MTHFS. A complementation assay in which wildtype human MTHFR activity complemented met11 deficiency (Shan et al., JBC, 274:32613-32618, 1999) described, was not highly sensitive and could not detect all functionally impaired human MTHFR alleles. For example, the assay was not capable of distinguishing between wildtype MTHFR and the functionally impaired common polymorphism A222V. Further, this assay revealed nothing about the relationship between folate levels and enzyme activity.

The in vivo assays disclosed herein are highly sensitive and capable of unmasking impaired alleles of genes involved in folate/homocysteine metabolism, as demonstrated herein with respect to MTHFR, while simultaneously determining the sensitivity thereof to folate. The alleles identified include low frequency alleles, dominant or codominant alleles that exhibit phenotypes as heterozygotes, alleles that are folate-sensitive, including alleles that are folate remediable, and alleles which possess combinations of these characteristics. Importantly, these impaired alleles are associated with the risk of a variety of conditions and diseases, as well as the varied efficacy and toxicity of chemotherapeutic agents. The deficiency of these impaired alleles may not manifest as a condition, disease, or varied response to chemotherapy in some individuals due to the compensatory effect of folate availability. The ability to unmask functionally impaired alleles of MTHFR provides for methods of screening for a risk of such conditions and diseases, as well as for the potential therapeutic efficacy and toxicity of chemotherapeutics.

The invention also provides in vivo assays for detecting impaired alleles of CTH and CBS. The ability to unmask functionally impaired alleles of these genes similarly provides for methods of screening for risk of associated diseases and conditions.

Accordingly, in one aspect, the invention provides in vivo assays for detecting impaired alleles of enzyme-encoding genes in metabolic pathways, and determining their sensitivity to cofactors. The assays comprise the use of yeast strains that comprise a first mutation in a first gene that may be complemented by the wildtype enzyme-encoding gene, and a second mutation in a second gene (or group of genes) that renders the yeast strain dependent on supplementation with the cofactor (or precursor thereof) for an assayable phenotype related to function of the first gene.

The methods comprise (i) introducing into a yeast cell a test allele of an enzyme-encoding gene, wherein the yeast cell comprises a first mutation in a first gene that is functionally homologous to the enzyme-encoding gene, and a second mutation in a second gene (or group of genes) that renders the yeast cell dependent upon supplementation with a cofactor required for enzyme function, wherein the first mutation alters a measurable characteristic of the yeast related to the function of the first gene; (ii) supplementing the growth medium with the cofactor; and (iii) detecting less restoration of the measurable characteristic in the presence of the test allele than in the presence of the wildtype enzyme, thereby detecting incomplete complementation of the first gene mutation by the test allele and identifying the test allele as an impaired allele. By titrating the amount of supplemented cofactor, the sensitivity of the impaired allele to cofactor availability is determined.

In one embodiment, diploid yeast cells are used. The diploid yeast may be homozygous or heterozygous for a test allele. Diploid yeast may comprise a wildtype gene and a test allele. Diploid yeast may comprise a combination of test alleles.

In a preferred embodiment, the enzyme-encoding gene corresponds in sequence to a naturally occurring allele, or to a compilation of individual naturally occurring alleles. In a preferred embodiment, the enzyme-encoding gene comprises an allele of a human enzyme-encoding gene, or a compilation of individual human alleles.

In a preferred embodiment, the yeast is S. cerevisiae.

In one embodiment, the first yeast gene is met13 and the second yeast gene is fol3. Such a yeast strain may be used to determine the activity of MTHFR alleles, and the response thereof to folate status. Accordingly, in one embodiment, the invention provides in vivo assays for determining the activity of MTHFR alleles, which are further capable of determining activity as a function of folate status. In a preferred embodiment, the enzyme-encoding gene comprises a naturally occurring human MTHFR allele. In another preferred embodiment, the enzyme-encoding gene comprises a compilation of individual human MTHFR alleles.

In a preferred embodiment, the assay method comprises comparing the activity of an MTHFR allele of interest to that of wildtype MTHFR.

In a preferred embodiment, the assay method comprises titrating the amount of folate to determine whether an MTHFR enzyme is sensitive to folate availability.

In one embodiment, the yeast is diploid. In one embodiment, the diploid yeast is heterozygous with respect to the MTHFR allele being tested for complementation. In one embodiment, the diploid yeast comprises wildtype MTHFR and a mutant MTHFR allele.

In a preferred embodiment, the measured output of the assay is growth.

In one embodiment, the first yeast gene is ade16 or ade17 and the second yeast gene is foI3. Such a yeast strain may be used to determine the activity of bifunctional enzyme AICAR Transformylase and IMP Cyclohydrolase (ATIC) alleles, and the response thereof to folate status. Accordingly, in one embodiment, the invention provides in vivo assays for determining the activity of ATIC alleles, which are further capable of determining activity as a function of folate status. In a preferred embodiment, the enzyme-encoding gene comprises a naturally occurring human ATIC allele. In another preferred embodiment, the enzyme-encoding gene comprises a compilation of individual human ATIC alleles.

In one embodiment, the first yeast gene is ade7 and the second yeast gene is fol3. Such a yeast strain may be used to determine the activity of glycinamide ribonucleotide transformylase (GART) alleles, and the response thereof to folate status. Accordingly, in one embodiment, the invention provides in vivo assays for determining the activity of GART alleles, which are further capable of determining activity as a function of folate status. In a preferred embodiment, the enzyme-encoding gene comprises a naturally occurring human GART allele. In another preferred embodiment, the enzyme-encoding gene comprises a compilation of individual human GART alleles.

In one embodiment, the first yeast gene is sam1 or sam2 and the second yeast gene is fol3. Such a yeast strain may be used to determine the activity of methionine adenosyltransferase I, alpha (MAT1A) alleles, and the response thereof to folate status. Accordingly, in one embodiment, the invention provides in vivo assays for determining the activity of MAT1A alleles, which are further capable of determining activity as a function of folate status. In a preferred embodiment, the enzyme-encoding gene comprises a naturally occurring human MAT1A allele. In another preferred embodiment, the enzyme-encoding gene comprises a compilation of individual human MAT1A alleles.

In one embodiment, the first yeast gene is sam1 or sam2 and the second yeast gene is fol3. Such a yeast strain may be used to determine the activity of methionine adenosyltransferase II, alpha (MAT2A) alleles, and the response thereof to folate status. Accordingly, in one embodiment, the invention provides in vivo assays for determining the activity of MAT2A alleles, which are further capable of determining activity as a function of folate status. In a preferred embodiment, the enzyme encoding gene comprises a naturally occurring human MAT2A allele. In another preferred embodiment, the enzyme-encoding gene comprises a compilation of individual human MAT2A alleles.

In one embodiment, the first yeast gene is fau1 and the second yeast gene is fol3. Such a yeast strain may be used to determine the activity of methenyltetrahydrofolate synthetase (MTHFS) alleles, and the response thereof to folate status. Accordingly, in one embodiment, the invention provides in vivo assays for determining the activity of MTHFS alleles, which are further capable of determining activity as a function of folate status. In a preferred embodiment, the enzyme-encoding gene comprises a naturally occurring human MTHFS allele. In another preferred embodiment, the enzyme-encoding gene comprises a compilation of individual human MTHFS alleles.

In another embodiment, the first yeast gene is cys3, and the second group of yeast genes is sextuple-delete sno1Δ sno2Δ sno3Δ snz1Δ snz2Δ snz3Δ. Such a yeast strain may be used to determine the activity of CTH alleles, and the response thereof to vitamin B₆ status. Accordingly, in one embodiment, the invention provides in vivo assays for determining the activity of CTH alleles, which are further capable of determining activity as a function of vitamin B₆ status. In a preferred embodiment, the enzyme-encoding gene comprises a naturally occurring human CTH allele. In another preferred embodiment, the enzyme-encoding gene comprises a compilation of individual human CTH alleles.

In another embodiment, the first yeast gene is cys4, and the second group of yeast genes is sextuple-delete sno1Δ sno2Δ sno3Δ snz1Δ snz2Δ snz3. Such a yeast strain may be used to determine the activity of CBS alleles, and the response thereof to vitamin B₆ status. Accordingly, in one embodiment, the invention provides in vivo assays for determining the activity of CBS alleles, which are further capable of determining activity as a function of vitamin B₆ status. In a preferred embodiment, the enzyme-encoding gene comprises a naturally occurring human CBS allele. In another preferred embodiment, the enzyme-encoding gene comprises a compilation of individual human CBS alleles.

In one aspect, the invention provides yeast strains capable of detecting impaired alleles of genes involved in folate/homocysteine metabolism and the sensitivity thereof to cofactors.

In one embodiment, the invention provides yeast strains capable of detecting impaired alleles of enzyme-encoding genes selected from the group consisting of ATIC, GART, MAT1A, MAT2AMTHFR, and MTHFS, and determining the responsiveness thereof to folate. In some embodiments, the yeast comprises the respective mutations and additions described hereinabove for each such enzyme-encoding gene.

In one embodiment, the invention provides yeast strains capable of detecting impaired alleles of CTH and determining the responsiveness thereof to vitamin B₆.

In one embodiment, the invention provides yeast strains capable of detecting impaired alleles of CBS and determining the responsiveness thereof to vitamin B₆.

In one aspect, the invention provides methods for detecting an impaired allele of an enzyme encoding gene in a metabolic pathway, such as, e.g. folate/homocysteine metabolism. In one embodiment, the impaired allele(s) are naturally-occurring in human ATIC, GART, MAT1A, MAT2A, MTHFR, and/or MTHFS. In one embodiment, the impaired allele is a CBS allele. In one embodiment, the impaired allele is a CTH allele. In some embodiments, the methods comprise detecting an impaired allele in a metabolic enzyme-encoding gene which has been shown to be cofactor-remediable using the in vivo assays and methods provided herein.

In another aspect, the invention provides methods for identifying and/or characterizing a metabolic enzyme deficiency in a subject, comprising obtaining a sample from the subject and detecting the presence or absence of a plurality of impaired alleles in said sample, wherein the presence of at least one impaired allele indicates that the subject is at risk of an enzyme deficiency. The plurality of impaired alleles may be from the same enzyme-encoding gene in the metabolic pathway, or may be alleles from multiple genes in the same pathway.

In some embodiments, one or more of the impaired alleles are low-frequency alleles, e.g., generally expressed in less than 4% of the general population, more generally in less than 3% of the general population, preferably less than 2.5% to 2%, and most preferably in less than 1% of the general population. In some embodiments, one or more of the impaired alleles are cofactor remediable alleles. In particularly preferred embodiments, the cofactor-remediable impaired alleles are identified by the in vivo assays and methods provided herein.

In another aspect, methods for detecting a predisposition to a cofactor-dependent enzyme deficiency in a subject are provided, comprising obtaining a sample from the subject and detecting the presence or absence of a plurality of impaired alleles in said sample, wherein the presence of at least one impaired allele indicates that the subject may have a remediable enzyme deficiency. The plurality of impaired alleles may be from the same enzyme-encoding gene in the metabolic pathway, or may be alleles from multiple genes in the same pathway.

In some embodiments, one or more of the impaired alleles are low-frequency alleles, e.g., generally expressed in less than 4% of the general population, more generally in less than 3% of the general population, preferably less than 2.5% to 2%, and most preferably in less than 1% of the general population. In some embodiments, one or more of the impaired alleles are cofactor remediable alleles. In particularly preferred embodiments, the cofactor-remediable impaired alleles are identified by the in vivo assays and methods provided herein.

The detection of specific alleles in samples is common in the art and any conventional detection protocol may be advantageously employed in the subject methods including protocols based on, e.g., hybridization, amplification, sequencing, RFLP analysis, and the like, as described herein. Also contemplated for use herein are protocols and/or materials developed in the future having particular utility in the detection of alleles in nucleic acid samples.

In a further aspect, methods for treating a metabolic enzyme deficiency in a subject are provided, comprising obtaining a sample from a subject having or suspected of having such a deficiency, detecting the presence or absence of a plurality of cofactor-remediable impaired alleles in the sample, and administering an appropriate cofactor supplement to the subject based on the number and type of impaired allele(s) detected in the sample, as described herein.

In one embodiment, the methods further comprise use of an in vivo assay for determining enzyme activity, as described herein,

In one embodiment, the methods further comprise use of an in vivo assay for determining enzyme activity, as described herein, and detecting a mutation in an enzyme-encoding nucleic acid.

In one embodiment, the methods further comprise use of an in vivo assay for determining enzyme activity, as described herein, and a temperature sensitivity assay to determine enzyme stability at an elevated temperature.

In one embodiment, the methods further comprise use of an in vivo assay for determining enzyme activity, as described herein, and an in vitro assay for determining the specific activity of the enzyme.

In one aspect, the invention provides methods of screening for risk of a disease or condition associated with aberrant homocysteine metabolism. The methods comprise screening for an impaired allele of a gene involved in homocysteine metabolism, as disclosed herein. In a preferred embodiment, the methods comprise detecting an impaired allele which has been characterized as such using an in vivo assay described herein. In a preferred embodiment, the disease or condition is selected from the group consisting of cardiovascular disease, coronary artery disease, ischemic stroke, atherosclerosis, neural tube defects, orofacial clefts, pre-eclampsia, pre-term delivery/low birth weight, recurrent early spontaneous abortion, thrombosis, retinal artery occlusion, down's syndrome, colorectal cancer, breast cancer, lung cancer, prostate cancer, depression, schizophrenia, Alzheimer's disease/dementia, age-related macular degeneration, and glaucoma

In one embodiment, the methods comprise screening for an impaired allele of ATIC, GART, MAT1A, MAT2A, MTHFR, and/or MTHFS, as described herein.

In one embodiment, the methods comprise screening for an impaired allele of CBS, as described herein.

In one embodiment, the methods comprise screening for an impaired allele of CTH, as described herein.

In one aspect, the invention provides methods for determining the chemotherapeutic response potential of an individual. The methods comprise use of a method for detecting an impaired allele of a gene involved in folate/homocysteine metabolism, as described herein. In a preferred embodiment, the gene is selected from the group consisting of MTHFR, ATIC, MTHFS, MAT1A, MAT2A, and GART. Detection of an impaired allele in the individual by the in vivo assay methods described herein and/or by application of detection methods for specific alleles indicates a decreased response potential.

In one aspect, the invention provides methods of determining potential chemotherapeutic toxicity for an individual. The methods comprise use of a method for detecting an impaired allele of a gene involved in folate/homocysteine metabolism, as described herein. In a preferred embodiment, the gene is selected from the group consisting of MTHFR, ATIC, MTHFS, MAT1A, MAT2A, and GART. Detection of an impaired allele in the individual by the in vivo assay methods described herein and/or by application of detection methods for specific alleles indicates an increased toxicity potential.

In one aspect, the invention provides isolated nucleic acids corresponding in sequence to alleles of an enzyme-encoding gene selected from the group consisting of MTHFR ATIC, MTHFS, MAT1A, MAT2A, and GART. In one embodiment, the isolated nucleic acid has and/or comprises a sequence of an allele of an MTHFR gene, e.g., a SNP disclosed in Table A. In one embodiment, the isolated nucleic acid has and/or comprises a sequence of an allele of an ATIC gene, e.g., a SNP disclosed in Table B. In one embodiment, the isolated nucleic acid has and/or comprises a sequence of an allele of an MTHFS gene, e.g., a SNP disclosed in Table C. In one embodiment, the isolated nucleic acid has and/or comprises a sequence of an allele of an MAT1A gene, e.g., a SNP disclosed in Table D. In one embodiment, the isolated nucleic acid has and/or comprises a sequence of an allele of an MAT2A gene, e.g., a SNP disclosed in Table E. In one embodiment, the isolated nucleic acid has and/or comprises a sequence of an allele of an GART gene, e.g., a SNP disclosed in Table F. In one embodiment, the nucleic acid corresponds to a sequence of an MTHFR allele and comprises a sequence encoding a non-synonymous mutation in the MTHFR protein selected from the group consisting of M110I, H213R, D223N, D291N, R519C, R519L, and Q648P.

In one aspect, the invention provides arrays for detecting impaired alleles of genes involved in folate/homocysteine metabolism.

In one embodiment, the invention provides arrays for detecting an impaired allele of a gene selected from the group consisting of ATIC, GART, MAT1A, MAT2A, MTHFR and MTHFS. In a preferred embodiment, the array is capable of detecting more than one impaired allele for a gene selected from the group. In a preferred embodiment, the array is capable of detecting more than one impaired allele a plurality of genes selected from the group. In one embodiment, the array is capable of detecting more than one impaired allele from each of a plurality of genes selected from the group. In a preferred embodiment, the array is capable of detecting such an impaired allele that is a remediable impaired allele. In a preferred embodiment, the array is capable of detecting a plurality of such impaired alleles that are remediable impaired alleles. In some embodiments, at least one of the impaired alleles is a low-frequency allele.

In one embodiment, the invention provides arrays for detecting an impaired MTHFR allele. In one embodiment, the array comprises one or more nucleic acids capable of hybridizing to an MTHFR allele comprising a non-synonymous mutation selected from the group consisting of those encoding M1101, H213R, D223N, D291N, R519C, R519L, and Q648P.

In one embodiment, the invention provides arrays for detecting impaired alleles of CBS. The arrays comprise one or more nucleic acids capable of hybridizing to an impaired allele of CBS.

In one embodiment, the invention provides arrays for detecting impaired alleles of CTH. The arrays comprise one or more nucleic acids capable of hybridizing to an impaired allele of CTH.

In a preferred embodiment, the invention provides arrays for detecting impaired alleles of a plurality of genes involved in folate/homocysteine metabolism. The arrays of the invention may use any of the many array, probe and readout technologies known in the art.

In one aspect, the invention provides a method of preventing a condition or disease associated with aberrant folate/homocysteine metabolism in an individual harboring a remediable impaired allele of a gene involved in folate/homocysteine metabolism. In one embodiment, the method comprises increasing the individual's intake of folate. In one embodiment, the method comprises increasing the individual's intake of vitamin B₆. In a preferred embodiment, the method comprises a method of screening for risk of a disease or condition associated with aberrant folate/homocysteine metabolism, as described herein.

In one aspect, the invention provides a method of treating a condition or disease associated with aberrant folate/homocysteine metabolism wherein the patient harbors a remediable impaired allele of a gene involved in folate/homocysteine metabolism. In one embodiment, the method comprises increasing the patient's intake of folate. In one embodiment, the method comprises increasing the individual's intake of vitamin B₆. In a preferred embodiment, the method comprises a method of screening for risk of a disease or condition associated with aberrant folate/homocysteine metabolism, as described herein.

In one aspect, the invention provides a method of increasing the chemotherapeutic response potential of an individual harboring a remediable impaired allele of a gene involved in folate/homocysteine metabolism. The method comprises increasing the individual's intake of folate. In a preferred embodiment, the method comprises a method of screening for risk of a disease or condition associated with aberrant folate/homocysteine metabolism, as described herein. In a preferred embodiment, the gene is selected from the group consisting of MTHFR, ATIC, MTHFS, MAT1A, MAT2A, and GART.

In one aspect, the invention provides a method of decreasing the toxicity of a chemotherapeutic for an individual harboring a remediable impaired allele of a gene involved in folate/homocysteine metabolism. The method comprises increasing the individual's intake of folate. In a preferred embodiment, the method comprises a method of screening for risk of a disease or condition associated with aberrant folate/homocysteine metabolism, as described herein. In a preferred embodiment, the gene is selected from the group consisting of MTHFR, ATIC, MTHFS, MAT1A, MAT2A, and GART.

In another aspect, the present invention provides a formulation comprising a cofactor, wherein said cofactor is present in an amount determined by the genetic makeup of an individual. The formulation of the present invention can comprise a plurality of cofactors, wherein at least a subset of said cofactors within said plurality is present in an amount determined by the genetic makeup of an individual. In one embodiment, the cofactor is selected from the group consisting of: Vitamin A (retinol), Vitamin C (ascorbic acid), Vitamin D (calciferol), Vitamin E, Vitamin K (phylloquinone), Vitamin B1 (Thiamin), Vitamin B2 (riboflavin), Vitamin B3 (niacin), Vitamin B6 (pyridoxine), Vitamin B9 (folate/folic acid), Vitamin B12 (tocopherol), Vitamin B7 (biotin), Vitamin B5 (panthothenic acid), and choline. In another embodiment, said plurality of cofactors comprises at least 2 cofactors selected from the group consisting of: Vitamin A (retinol), Vitamin C (ascorbic acid), Vitamin D (calciferol), Vitamin E, Vitamin K (phylloquinone), Vitamin B1 (Thiamin), Vitamin B2 (riboflavin), Vitamin B3 (niacin), Vitamin B6 (pyridoxine), Vitamin B9 (folate/folic acid), Vitamin B12 (tocopherol), Vitamin B7 (biotin), Vitamin B5 (panthothenic acid), and choline.

In some embodiments, the formulation of the present invention can be prepared as a sustained release form. In other embodiments, the formulation of the present invention is orally ingestible. The formulation can be as a unit dosage, in form of a tablet or a capsule, or in liquid form. The formulation can also be prepared for intravenous, subcutaneous, or intramuscular administration. Where desired, the formulation of the present invention can be accompanied by instructions for use by said individual.

In yet another aspect, the present invention provides a method of preparing a formulation comprising: (a) selecting a cofactor, wherein said cofactor is present in an amount determined by genetic makeup of an individual; and (b) mixing said cofactor with an excipient in an ingestible or injectable form. In one embodiment, the step of selecting comprises selecting a plurality of cofactors, wherein at least a subset of said cofactors within said plurality is present in an amount determined by the genetic makeup of said individual. In another embodiment, said cofactor is selected based on at least one personal characteristic of said individual, wherein said personal characteristic is selected from the group consisting of: weight, height, body-mass index, ethnicity, ancestry, gender, age, family history, medical history, exercise habit, and dietary habit.

In a related but separate aspect, the present invention provides a method of determining a risk or predisposition to a cofactor remediable condition in an individual comprising: (a) detecting the presence or absence of a plurality of genetic variants from a biological sample of said individual, wherein said plurality of genetic variants is selected from Tables A-X; and (b) determining said predisposition to said cofactor remediable condition when said plurality of genetic variants is detected in said biological sample. In some embodiments, the plurality of genetic variants comprises at least 2, 3, 4, 5, 5, 7, 8, 9, 10, 20, 30, 40, 50, 100, 150, 200, 300, 400, 500 or more genetic variants. In other embodiments, the subject method further comprising reporting said risk of a cofactor-dependent enzyme deficiency to said individual or a health care manager of said individual.

In yet another aspect, the present invention provides a method of determining an amount of cofactor for an individual comprising: (a) detecting the presence or absence of at least one genetic variant from a biological sample of said individual, wherein said at least one genetic variant correlates to a recommended amount of a cofactor that differs by at least 1% of the mass of said cofactor as compared to an amount recommended to an individual lacking said at least one genetic variant; and (b) recommending said different amount of cofactor for said individual when said at least one genetic variant is detected in said biological sample. In some embodiments, the genetic variant correlates to a recommended amount of a cofactor that differs by at least 1% greater than an amount recommended to an individual lacking said at least one genetic variant. In other embodiments, the genetic variant correlates to a recommended amount of a cofactor that differs by at least 1% less than an amount recommended to an individual lacking said at least one genetic variant. In yet other embodiments, the genetic variant correlates to a recommended amount of a cofactor that differs by at least 500%. The individual can be a female with a risk or predisposition for a cofactor remediable condition.

The present invention further provides an isolated nucleic acid or a complement thereof, wherein said nucleic acid comprises'a single nucleotide polymorphism (SNP) shown in Table A-X. Also contemplated is an array comprising immobilized thereon a plurality of isolated nucleic acids of the present invention.

The present invention also provides a computer assisted method of providing a personalized nutritional advice plan for an individual comprising: (i) providing a first dataset on a data processing device, said first dataset comprising information correlating the presence of genetic variant of said individual, wherein the genetic variant indicates that the individual is at risk of a cofactor-dependent enzyme deficiency; (ii) providing a second dataset on a data processing device, said second dataset comprising information matching said co-factor-dependent enzyme deficiency with at least one lifestyle recommendation; and (iii) generating a personalized nutritional advice plan based on the genetic variant of (i), wherein the plan comprises at least one lifestyle recommendation matched in step (ii). In some embodiments, said personalized lifestyle advice plan includes recommended minimum and/or maximum amounts of vitamin subtypes. In some embodiments, the first data set comprises a plurality of genetic variants selected from Tables A-X. In other embodiments, the personalized lifestyle advice plan includes recommended one or more cofactor in an amount based on the genetic variant of said individual. Where desired, the method comprises the step of delivering the plan to the individual via Internet with the use of a unique identifier code. Such delivery can be done wirelessly to the individual or his/her agent, e.g., via an I-Phone®. In some embodiments, the plan comprises hyperlinks to one or more Web pages. In some other embodiments, the one or more cofactor-dependent enzyme deficiencies analyzed by the subject method is folate/folic acid deficiency. In other embodiments, the computer-assisted method encompasses a third dataset on a data processing device, said third dataset comprising information on one or more personal characteristics of said individual. The personal characteristic(s) includes but is not limited to weight, height, body-mass index, ethnicity, ancestry, gender, age, family history, medical history, exercise habit, and dietary habit. In practice the method, the step of providing the first dataset of (i) and/or providing the second dataset of (ii) can be carried out by inputting information of respective dataset by said individual or his/her agent.

The present invention further provides a computer system comprising (i) a data processing device configured to process a first dataset and/or a second data set, said first dataset comprising information correlating the presence of genetic variant of an individual, wherein the genetic variant indicates that the individual is at risk of a cofactor-dependent enzyme deficiency, and said second dataset comprising information matching said co-factor-dependent enzyme deficiency with at least one lifestyle recommendation; and (ii) an output device configured to generate a personalized nutritional advice plan based on the genetic variant of said individual, wherein the plan comprises at least one lifestyle recommendation matched in (i). The computer system provided herein can further comprise an input device configured for inputting information on first data set and/or second data set. In some embodiments, the input device is configured to input information on one or more personal characteristics of said individual.

Also provided in the present invention is a business method of providing a personalized nutritional advice plan for an individual, comprising: collecting information concerning the presence or absence of at least one genetic variant from a biological sample of said individual, wherein said at least one genetic variant correlates to a recommended amount of a cofactor that differs by at least 1% of said cofactor as compared to an amount recommended to an individual lacking said at least one genetic variant; and recommending said different amount of cofactor for said individual when said at least one genetic variant is detected in said biological sample.

The methods contemplated herein encompass the aspect where the genetic variant correlates to a recommended amount of a cofactor that differs by at least 1%, 5%, 10%, 100%. 500%, 1000% greater than an amount recommended to an individual lacking said at least one genetic variant. The subject methods also contemplate the aspect where the genetic variant correlates to a recommended amount of a cofactor that differs by at least 1%, 5%, 10%, 100%. 500%, 1000% less than an amount recommended to an individual lacking said at least one genetic variant. The inventions disclosed herein also encompass cofactor remediable condition including but not limited to having an offspring with a neural tube defect (e.g., spina bifida), cleft palate, or anencephaly, or having a preterm birth. In some embodiments, the individual of interest is a pregnant female and said cofactor remediable condition is having an offspring with spina bifida.

### BRIEF DESCRIPTION OF THE DRAWINGS

FIG. 1. Effects of folinic acid supplementation on growth rate of fol3Δ::KanMX cells and cellular activity of human MTHFR. (a) Growth of fol3Δ::KanMX MET13 haploid yeast was measured in 96-well plates as described in Materials and Methods. Media was supplemented with folinic acid at the indicated concentrations. The curve labeled FOL3 (FOL3 MET13) was from growth in medium without folinic acid. (b) Growth of fol3Δ::KanMX met13Δ::KanMX haploid yeast transformed with phMTHFR in media lacking methionine and supplemented with folinic acid at the indicated concentrations. 3 independent transformants were tested at each folinic acid concentration to test reproducibility. The curve labeled met3Δ represented a single isolate of cells, transformed with empty vector, grown at 50 ug/ml folinic acid.

FIG. 2. Functional impact and folate-remediability of nonsynonymous MTHFR population variants. (a>6 MTHFR variants were tested for the ability to rescue fol3Δ::KanMX met13Δ::KanMX cells in media lacking methionine at 3 different folinic acid concentrations. The M1101 allele and the M1101A222V doubly-substituted allele were tested only at 50 and 25 ug/ml folinic acid. The curve labeled Major corresponds to the most common MTHFR allele in the population. Each curve is from a pool of 3-6 independent transformants. (b) Schematic of the MTHFR protein (656 amino acids>divided into a N-terminal catalytic domain and a C-terminal regulatory domain of nearly equal size (35), Positions of all nonsynonymous changes are indicated. Benign changes are in green. Changes numbered 1 through 4 represent folate-remedial alleles indicated in increasing order of severity. Change #5 (R134C) was nearly loss-of-function and not designated as folate-remedial (see Results) but was somewhat folate-augmentable.

FIG. 3. Enzyme activity of MTHFR variants. Crude yeast extract from cells transformed with the indicated MTHFR constructs was prepared and assayed for MTHFR activity as described herein. Heat treatment for the indicated times was done on reactions prior to addition of radiolabeled substrate. Measurements were averages of two independent sets of triplicate assays; error bars are standard deviation for the 6 data points.

FIG. 4. Heterozygote phenotypes for MTHFR variants as recapitulated in yeast. Homozygosity or heterozygosity of MTHFR alleles was recreated in diploid yeast for the major, R134C and A222V alleles as described herein. Diploids were obtained from the mating of haploid strains that each expressed a single allele of MTHFR integrated in the genome. Growth as a function of folinic acid supplementation was assayed exactly as for haploids.

FIG. 5. Immunoblot of human MTHFR variants expressed in yeast. (a) Extracts were made from yeast cells carrying different MTHFR alleles and detected with anti-HA antibody as described herein. A222V M110I was a doubly substituted allele; Major indicates the most common MTHFR allele in the population. The two right-most lanes were, side-by-side, the major allele and the non-phosphorylatable T34A allele (37). (b) The ratio of signal intensities of the unphosphorylated lower band to the phosphorylated upper band for all variants of MTHFR identified in this study plotted as a function of increasing severity of functional impact. Alleles on the x-axis were classified as benign or rank-ordered with respect to activity. All benign alleles (including the Major allele and all regulatory domain changes) were plotted and show nearly identical ratios of the two MTHFR species, thus the symbols overlapped.

FIG. 6. Assays for B₆ (pyridoxine)-responsiveness in two human B enzymes: CBS and CTH.

FIG. 7. A schematic of an exemplary system for analyzing the genetic makeup of an individual and determining the cofactor formulation, the risk or predisposition of a cofactor remediable condition, or both, for the individual.

### DETAILED DESCRIPTION

As indicated above, the present invention provides in vivo assays for identifying impaired alleles of enzyme-encoding genes within metabolic pathways and determining their sensitivity to cofactor remediation. Compound yeast mutants, comprising a first mutation allowing for complementation by a functionally homologous enzyme of interest, and a second mutation (or group of mutations>rendering the strain dependent upon supplementation with a cofactor, provide for the study of enzyme complementation as a function of cofactor availability. Significantly, the present invention also demonstrates that cofactor remediation of low-frequency impaired alleles in enzyme-encoding genes is surprisingly common, and that these alleles can collectively have a significant impact on the metabolic pathway. Accordingly, the present invention contemplates diagnostic and prognostic methods focused in particular on the detection and characterization of such low-frequency impaired alleles in enzyme-encoding genes, and determination of their effective remediation.

The “N-terminal catalytic domain” of MTHFR refers to amino acids 1-359 in human MTHFR. The reference human MTHFR mRNA sequence is found at Genbank accession no. NM 005957, while the encoded 656 amino acid sequence is found at Genbank accession no. NP005958.

By MTHFR dysfunction is meant a deviation from wildtype MTHFR activity. Enzyme dysfunction and associated conditions and diseases can arise through, for example, changes in the specific activity of an enzyme, mislocalization of an enzyme, changes in the level of an enzyme, and other changes.

In Vivo Assays for Measuring Enzyme Activity and Sensitivity Thereof to Cofactors

The assays provided herein may be used to test the ability of alleles of genes encoding enzymes to complement mutations in functionally homologous yeast genes, as well to measure the responsiveness of these enzymes to cofactors. The assays comprise measuring an output, or phenotype, that is associated with normal function of the yeast gene and altered by its dysfunction.

The assays comprise the use of yeast strains that comprise a first mutation allowing for complementation by a functionally homologous enzyme of interest, and a second mutation rendering the strain dependent upon supplementation with cofactor for an assayable phenotype related to function of the first gene.

The methods comprise (i) introducing into a yeast cell a test allele of an enzyme-encoding gene, wherein the yeast cell comprises a first mutation in a first gene that is functionally homologous to the enzyme-encoding gene, and a second mutation in a second gene (or group of genes) that renders the yeast cell dependent upon supplementation with a cofactor required for enzyme function, wherein the first mutation alters a measurable characteristic of the yeast related to the function of the first gene; (ii) supplementing the growth medium with the cofactor; and (iii) detecting less restoration of the measurable characteristic in the presence of the test allele than in the presence of the wildtype enzyme, thereby detecting incomplete complementation of the first gene mutation by the test allele and identifying the test allele as an impaired allele. By varying the amount of supplemented cofactor, the sensitivity of the impaired allele to cofactor availability is determined,

In a preferred embodiment, the test allele of an enzyme-encoding gene corresponds in sequence to a naturally occurring allele, or to a compilation of individual naturally occurring polymorphisms. In a preferred embodiment, the test allele corresponds in sequence to an allele of a human gene, or to a compilation of individual polymorphisms in a plurality of human alleles.

In a preferred embodiment, the yeast is Saccharomyces cerevisiae (“S. cerevisiae”), though other species of yeast may be used.

In one embodiment, diploid yeast are used. The diploid yeast may be homozygous or heterozygous for a test allele. Diploid yeast may comprise a wildtype gene and a test allele. Diploid yeast may comprise a combination of test alleles. As demonstrated herein, functionally impaired alleles may include alleles having a heterozygous phenotype. In one embodiment, the diploid yeast is heterozygous with respect to the allele being tested for complementation. In one embodiment, the diploid yeast comprises a wildtype allele and an impaired allele of an enzyme-encoding gene.

In a preferred embodiment, the measured output of the assay is growth.

In a preferred embodiment, the assay method comprises comparing the activity of a test allele of interest to that of a corresponding wildtype allele.

In one embodiment, the invention provides in vivo assays for determining the activity of a test allele, e.g., an allele of an enzyme-encoding gene. In one embodiment, the enzyme-encoding gene is involved in or related to folate/homocysteine metabolism. In another embodiment, the test allele is selected from the group consisting of an MTHFR allele, ATIC allele, GART allele, an MAT1A allele, an MAT2A allele, and an MTHFS allele, which assays are further capable of determining activity as a function of folate status. In another embodiment, the enzyme-encoding allele is selected from the group consisting of a CTH allele and CBS allele.

In one embodiment, the test allele is an MTHFR allele and comprises at least one substitution in the N-terminus catalytic domain and at least one mutation in the C-terminus regulatory region. While substitutions in the C-terminus region alone do not typically impair function, they may combine with other substitutions to functionally impair an allele,

In a preferred embodiment, the first mutation is in the yeast gene met13, which may be functionally complemented by wildtype human MTHFR. In another embodiment, the first yeast gene is ade16 or ade17, which may be functionally complemented by wildtype human ATIC. In one embodiment, the first yeast gene is ade7, which may be functionally complemented by wildtype human GART. In one embodiment, the first yeast gene is sam1 or sam2, which may be functionally complemented by wildtype human MAT1A or wildtype human MAT2A. In one embodiment, the first yeast gene is faul, which may be functionally complemented by wildtype human MTHFS.

In a preferred embodiment, the second mutation is in the yeast gene fol3, which renders the yeast dependent upon folate in supplemented medium. Such a yeast strain may be used to determine the activity of a test allele, the test allele depending on the first mutation, and the response thereof to folate status. For example, a compound yeast having a first mutation in the yeast gene met1, and a second mutation in the yeast gene fol3, may be used to determine the activity of an MTHFR allele and the response thereof to folate status.

In a preferred embodiment, the assay method comprises varying the amount of folate to determine whether the enzyme encoded by the test allele is sensitive to folate availability. In a preferred embodiment, the assay method includes measuring output in the presence of less than 50 ug/ml folate. In a preferred embodiment, the assay method includes measuring output in the presence of about 50 ug/ml folate. In a preferred embodiment, the assay method includes measuring output in the presence of more than 50 ug/ml folate.

In one embodiment, the folate is varied to determine whether an impaired allele of an enzyme-encoding gene is remediable by folate.

In another embodiment, the first yeast gene is cys3, and the second yeast gene is sextuple-delete sno1Δ sno2Δ sno3Δ snz1Δ snz2Δ snz3Δ. Such a yeast strain may be used to determine the activity of CTH alleles, and the response thereof to vitamin B₆ status. Accordingly, in one embodiment, the invention provides in vivo assays for determining the activity of CTH alleles, which are further capable of determining activity as a function of vitamin B₆ status. In a preferred embodiment, the CTH allele comprises a naturally occurring human allele. In another preferred embodiment, the CTH allele comprises a compilation of individual human CTH alleles.

In another embodiment, the first yeast gene is cys4, and the second yeast gene is sextuple-delete sno1Δ sno2Δ sno3Δ snz1Δ snz2Δ snz3Δ. Such a yeast strain may be used to determine the activity of CBS alleles, and the response thereof to vitamin B₆ status. Accordingly, in one embodiment, the invention provides in vivo assays for determining the activity of CBS alleles, which are further capable of determining activity as a function of vitamin B₆ status. In a preferred embodiment, the CBS allele comprises a naturally occurring human allele. In another preferred embodiment, the CBS allele comprises a compilation of individual human CBS alleles.

Table 1 below lists enzyme-encoding genes and provides exemplary compound yeast mutations that may be used to determine the activity of an allele of the enzyme-encoding gene.

TABLE 1
Enzyme-encoding genes and Yeast Backgrounds

| HGNC | Yeast Screening Strain Backgrounds |
|---|---|
| ATIC | fol3 ade16 ade17 |
| CBS | sno/snzl sno/snz2 sno/snz3 cys4 |
| CTH | sno/snzl sno/snz2 sno/snz3 cys3 |
| GART | fol3 ade8 |
| MAT1 A | fol3 sam1 sam2 |
| MAT2A | fol3 saml sam2 |
| MTHFR | fol3 met13 |
| MTHFS | fol3 faul |

Yeast strains may be generated by methods well known in the art. For example, see Shan et al, JBC, 274:32613-32618, 1999.

Introduction of nucleic acids into yeast strains may be done using methods well known in the art. For example, see Shan et al. JBC, 274:32613-32618, 1999.

Alleles of Enzyme-Encoding Genes

As described in the Examples section, single nucleotide polymorphisms that subtly affect enzymes, e.g., that result in an impaired allele of an enzyme-encoding gene may be characterized using the in vivo assay disclosed herein regardless of the frequency of the allele. For example, the methods disclosed herein were used to determine whether an allele is an impaired allele, and if so, whether the impaired allele is cofactor-remediable. Provided in Table 4 and Tables A-F are single nucleotide polymorphisms for the enzyme-encoding genes MTHFR, ATIC, MTHFS, MAT1A, MAT2A and GART that have been characterized (Table 4) or may be characterized (Tables A-F) by the assay described herein. These tables also provide SNPs for these genes which have not been previously identified. Accordingly, disclosed herein are alleles for an enzyme-encoding gene selected from the group consisting of MTHFR, ATIC, MTHFS, MAT1A, MAT2A, and GART. Also provided herein are genetic variants of the genes MTHFR, ATIC, MTHFS, MAT1A, MAT2A, and GART, as well as, AHCY, AMT, CBS, CTH, DHFR, FPGS, MTHFD1, MTHFD2, MTR, SHMT1, SHMT2, or TYMS, such as those listed in Tables A-X.

These alleles may be characterized using the assay disclosed herein, and may be advantageously detected in the methods of screening, preventing and treating as disclosed herein. An ordinarily skilled artisan will recognize and appreciate that characterization of an impaired allele as cofactor remediable informs the methods of screening, preventing and treating as disclosed herein.

As used herein, an “allele” is a nucleotide sequence, such as a single nucleotide polymorphism (SNP), present in more than one form in a genome. An “allele” as used herein is not limited to the naturally occurring sequence of a genomic locus. “Allele” includes transcripts and spliced sequence derived therefrom (e.g., mRNA sequence, cDNA sequence). An “allele” may be a naturally occurring allele or a synthetic allele. These may include mutations in the N-terminal catalytic domain as well as mutations in the C-terminal regulatory region.

“Homozygous”, according to the present invention, indicates that the two copies of the gene or SNP are identical in sequence to the other allele. For example, a subject homozygous for the wild-type allele of an enzyme-encoding gene contains at least two identical copies of the sequence. Such a subject would not be predisposed to a cofactor-dependent enzyme deficiency within a metabolic pathway.

“Heterozygous,” as used herein, indicates that two different copies of the allele are present in the genome, for example one copy of the wild-type allele and one copy of the variant allele, which may be an impaired allele. A subject having such a genome is heterozygous, and may be predisposed to a cofactor-dependent enzyme deficiency within a metabolic disease. “Heterozygous” also encompasses a subject having two different mutations in its alleles.

By “impaired allele” is meant an allele of a gene encoding a metabolic enzyme that is functionally impaired, which functional impairment may or may not be cofactor-remediable.

An “impaired allele mutation” refers to the particular nucleic acid mutation that underlies functional impairment of an impaired allele and distinguishes an impaired allele from wildtype sequence at the location of the mutation. Typically, an impaired allele mutation is a non-synonymous point mutation in a single codon.

“Cofactor-remediable” refers to the ability of altered cofactor level to compensate for the functional impairment of an impaired metabolic enzyme.

Supplementation with a cofactor includes supplementation with a precursor of a cofactor that may be converted to the cofactor.

“Cofactor” refers to factors that are direct cofactors of enzymes of interest (e.g., folate for MTHFR, ATIC, GART, MAT1A, MAT2A, and MTHFS), as well as factors that are indirect cofactors for enzymes of interest. Thus, cofactors can directly or indirectly impact enzyme function.

Measures of frequency known in the art include allele frequency, namely the fraction of genes in a population that have a specific SNP. The allele frequencies for any gene should sum to 1. Another measure of frequency known in the art is the “heterozygote frequency” namely, the fraction of individuals in a population who carry two alleles, or two forms of a SNP of a gene, one inherited from each parent. Alternatively, the number of individuals who are homozygous for a particular allele of a gene may be a useful measure. The relationship between allele frequency, heterozygote frequency, and homozygote frequency is described for many genes by the Hardy-Weinberg equation, which provides the relationship between allele frequency, heterozygote frequency and homozygote frequency in a freely breeding population at equilibrium. Most human variances are substantially in Hardy-Weinberg equilibrium. As used herein, a “low frequency allele” has an allele frequency of less than 4%.

Disclosed herein are alleles for human enzyme-encoding genes involved in or relevant to folate/homocysteine metabolism. By “folate/homocysteine metabolism” is meant folate and/or homocysteine metabolism. Such enzyme-encoding genes include MTHFR, ATIC, GART, MAT1A, MAT2A, MTHFS. The Hugo Gene Nomenclature Committee (HGNC) symbols, GeneIDs, NCBI nucleotide accession numbers (NC), NCBI polypeptide accession numbers (NB_) and names of enzyme-encoding genes involved in or relevant to folate/homocysteine metabolism is provided in Table 2.

TABLE 2
Human enzyme-encoding genes involved in or
relevant to folate/homocysteine metabolism

| | | NCBI | NCBI | |
|---|---|---|---|---|
| HGNC | GeneID | nucleotide | polypeptide | Name |
| ATIC | 471 | NC_000002.10 | NM_004044 | aminoimidazole-4-carboxamide |
| | | | | ribonucleotide formyltransferase/IMP |
| | | | | cyclohydrolase |
| GART | 2618 | NC_000021.7 | NM_000819 | glycinamide |
| | | | | ribonucleotidetransformylase |
| MAT1A | 4143 | NC_000010.9 | NM_000429 | methionine adenosyltransferase I, alpha |
| MAT2A | 4144 | NC_000002.10 | NM_005911 | methionine adenosyltransferase II, alpha |
| MTHFR | 4524 | NC_000001.9 | NM_005957 | methylenetetrahydrofolate reductase |
| MTHFS | 10588 | NC_000015.8 | NM_006441 | methenyltetrahydrofolate synthetase |

Other enzyme-encoding genes other than MTHFR, ATIC, GART, MAT1A, MAT2A, MTHFS, include AHCY, AHCYL1, AHCYL2, ALDH1L1, ALDHL2, AMT, BHMT1, BHMT2, CBS, CTH, DHFR, DMGDH, FPGS, GGH, MTFMT, MTHFD1, MTHFD2, MTR, MTRR, NAALAD2, SARDH, SHMT1, SHMT2, or TYMS, all of which are shown in Table 3. The genetic variants may be any of those listed in Tables A-X and they can be detected in the genetic makeup of an individual and used to select one or more cofactors, or the amount of one or more cofactors, for a formulation for that individual. As shown in Tables G-X, Polymorphism Phenotyping, (“PolyPhen,” see for example, http://genetics.bwh.harvard.edu/pph/), SIFT (Sorting Intolerant From Tolerant, see for example, Ng and Henikoff, Nucleic Acids Res. 2003 Jul. 1; 31(13): 3812-3814), MAF (Minor Allele Frequency) and HWE (Hardy Weinberg equilibrium) may be determined for a genetic variant. In some embodiments, the information from these can be used to provide information on the functional impact of a genetic variant, or used to determine the risk of a cofactor dependent enzyme deficiency or cofactor remediable condition. In some embodiments, the functional impact of a genetic variant can be determined by in vivo assays, such as yeast assays disclosed herein.

TABLE 3
Human enzyme-encoding genes involved in or
relevant to folate/homocysteine metabolism

| | | UniProt/ | |
|---|---|---|---|
| | | Swiss-Prot | |
| HGNC | EC | Accession | Cofactor/Coenzyme |
| AHCY | 3.3.1.1 | P23526 | Reduced Folate (indirectly) |
| AHCYL1 | 3.3.1.1 | O43865 | Reduced Folate (indirectly) |
| AHCYL2 | 3.3.1.1 | Q96HN2 | Reduced Folate (indirectly) |
| ALDH1L1 | 1.5.1.6 | O75891 | Reduced Folate |
| ALDH1L2 | 1.5.1.6 | Q3SY69 | Reduced Folate |
| AMT | 2.1.2.10 | P48728 | Reduced Folate |
| ATIC | 2.1.2.3 | P31939 | Reduced Folate |
| BHMT1 | 2.1.1.5 | Q93088 | Reduced Folate (indirectly) |
| BHMT2 | 2.1.1.5 | Q9H2M3 | Reduced Folate (indirectly) |
| CBS | 4.2.1.22 | P35520 | Pyridoxal-phosphate (B6) |
| CTH | 4.4.1.1 | P32929 | Pyridoxal-phosphate (B6) |
| DHFR | 1.5.1.3 | P00374 | Reduced Folate |
| DMGDH | 1.5.99.2 | Q9UI17 | Reduced Folate |
| FPGS | 6.3.2.17 | Q05932 | Reduced Folate |
| FTCD | 2.1.2.5 | O95954 | Reduced Folate |
| GART | 2.1.2.2 | P22102 | Reduced Folate |
| GGH | 3.4.19.9 | Q92820 | Reduced Folate |
| MAT1A | 2.5.1.6 | Q00266 | Reduced Folate (indirectly) |
| MAT2A | 2.5.1.6 | P31153 | Reduced Folate (indirectly) |
| MTFMT | 2.1.2.9 | Q96DP5 | Reduced Folate |
| MTHFD1 | 1.5.1.5 | P11586 | Reduced Folate |
| MTHFD2 | 1.5.1.15 | P13995 | Reduced Folate |
| MTHFR | 1.5.1.20 | P42898 | Reduced Folate |
| MTHFS | 6.3.3.2 | P49914 | Reduced Folate |
| MTR | 2.1.1.13 | Q99707 | Reduced Folate |
| MTRR | 1.16.1.8 | Q9UBK8 | Reduced Folate (indirectly) |
| NAALAD2 | 3.4.17.21 | Q9Y3Q0 | Reduced Folate |
| SARDH | 1.5.99.1 | Q9UL12 | Reduced Folate |
| SHMT1 | 2.1.2.1 | P34896 | Reduced Folate |
| SHMT2 | 2.1.2.1 | P34897 | Reduced Folate |
| TYMS | 2.1.1.45 | P04818 | Reduced Folate |

In one aspect, the invention provides isolated nucleic acids corresponding in sequence to human enzyme-encoding alleles involved in folate/homocysteine metabolism. For example, the invention provides isolated nucleic acids corresponding in sequence to an enzyme-encoding allele selected from the group consisting of an MTHFR allele, a ATIC allele, a GART allele, an MAT1A allele, an MAT2A allele, and an MTHFS allele, which may or may not be cofactor-remediable. These alleles include low frequency alleles. These alleles include impaired alleles. The allele can also be an AHCY, AHCYL1, AHCYL2, ALDH1L1, ALDHL2, AMT, BHMT1, BHMT2, CBS, CTH, DHFR, DMGDH, FPGS, FTCD, GGH, MTFMT, MTHFD1, MTHFD2, MTR, MTRR, NAALAD2, SARDH, SHMT1, SHMT2, or TYMS allele.

Accordingly, provided herein is an isolated nucleic acid corresponding in sequence to an allele of an MTHFR gene, wherein said nucleic acid comprises a SNP found at a nucleotide selected from the group consisting of nucleotide 4078 of the MTHFR gene; nucleotide 4234 of the MTHFR gene; nucleotide 5733 of the MTHFR gene; nucleotide 5872 of the MTHFR gene; nucleotide 6642 of the MTHFR gene; nucleotide 6657 of the MTHFR gene; nucleotide 6681 of the MTHFR gene; nucleotide 6774 of the MTHFR gene; nucleotide 10906 of the MTHFR gene; nucleotide 11656 of the MTHFR gene; nucleotide 11668 of the MTHFR gene; nucleotide 11902 of the MTHFR gene; nucleotide 12232 of the MTHFR gene; nucleotide 12622 of the MTHFR gene; nucleotide 12759 of the MTHFR gene; nucleotide 13040 of the MTHFR gene; nucleotide 14593 of the MTHFR gene; nucleotide 14612 of the MTHFR gene; nucleotide 14705 of the MTHFR gene; nucleotide 16170 of the MTHFR gene; nucleotide 16401 of the MTHFR gene; and nucleotide 16451 of the MTHFR gene. Examples of SNPs or genetic variants of MTHFR are provided in Tables A and S.

Also provided herein is an isolated nucleic acid corresponding in sequence to an allele of an ATIC gene, wherein said nucleic acid comprises a SNP found at a nucleotide selected from the group consisting of nucleotide 1100 of the ATIC gene; nucleotide 1114 of the ATIC gene; nucleotide 1179 of the ATIC gene; nucleotide 1244 of the ATIC gene; nucleotide 1270 of the ATIC gene; nucleotide 1288 of the ATIC gene; nucleotide 1301 of the ATIC gene; nucleotide 1380 of the ATIC gene; nucleotide 1396 of the ATIC gene; nucleotide 1453 of the ATIC gene; nucleotide 1506 of the ATIC gene; nucleotide 1689 of the ATIC gene; nucleotide 7227 of the ATIC gene; nucleotide 7232 of the ATIC gene; nucleotide 7388 of the ATIC gene; nucleotide 8756 of the ATIC gene; nucleotide 8808 of the ATIC gene; nucleotide 14099 of the ATIC gene; nucleotide 14140 of the ATIC gene; nucleotide 14144 of the ATIC gene; nucleotide 14183 of the ATIC gene; nucleotide 14229 of the ATIC gene; nucleotide 14238 of the ATIC gene; nucleotide 14245 of the ATIC gene; nucleotide 14260 of the ATIC gene; nucleotide 14489 of the ATIC gene; nucleotide 14970 of the ATIC gene; nucleotide 15003 of the ATIC gene; nucleotide 15040 of the ATIC gene; nucleotide 15043 of the ATIC gene; nucleotide 15149 of the ATIC gene; nucleotide 15240 of the ATIC gene; nucleotide 15844 of the ATIC gene; nucleotide 16063 of the ATIC gene; nucleotide 21363 of the ATIC gene; nucleotide 21372 of the ATIC gene; nucleotide 21400 of the ATIC gene; nucleotide 21521 of the ATIC gene; nucleotide 21611 of the ATIC gene; nucleotide 22187 of the ATIC gene; nucleotide 22273 of the ATIC gene; nucleotide 22282 of the ATIC gene; nucleotide 22291 of the ATIC gene; nucleotide 22342 of the ATIC gene; nucleotide 22512 of the ATIC gene; nucleotide 22519 of the ATIC gene; nucleotide 22538 of the ATIC gene; nucleotide 22564 of the ATIC gene; nucleotide 22589 of the ATIC gene; nucleotide 22737 of the ATIC gene; nucleotide 24992 of the ATIC gene; nucleotide 25009 of the ATIC gene; nucleotide 27757 of the ATIC gene; nucleotide 27855 of the ATIC gene; nucleotide 27985 of the ATIC gene; nucleotide 28015 of the ATIC gene; nucleotide 33901 of the ATIC gene; nucleotide 33919 of the ATIC gene; nucleotide 33920 of the ATIC gene; nucleotide 33933 of the ATIC gene; nucleotide 35723 of the ATIC gene; nucleotide 35737 of the ATIC gene; nucleotide 35742 of the ATIC gene; nucleotide 35840 of the ATIC gene; nucleotide 35917 of the ATIC gene; nucleotide 35968 of the ATIC gene; nucleotide 35973 of the ATIC gene; nucleotide 38338 of the ATIC gene; nucleotide 38342 of the ATIC gene; nucleotide 38437 of the ATIC gene; nucleotide 38342 of the ATIC gene; nucleotide 38582 of the ATIC gene; nucleotide 38627 of the ATIC gene; nucleotide 38667 of the ATIC gene; and nucleotide 38725 of the ATIC gene. Examples of SNPs or genetic variants of ATIC are provided in Tables B and I.

Also provided herein is an isolated nucleic acid corresponding in sequence to an allele of an MTHFS gene, wherein said nucleic acid comprises a SNP found at a nucleotide selected from the group consisting of nucleotide 8808 of the MTHFS gene; nucleotide 8912 of the MTHFS gene; nucleotide 8957 of the MTHFS gene; nucleotide 8998 of the MTHFS gene; nucleotide 52560 of the MTHFS gene; nucleotide 52878 of the MTHFS gene; and nucleotide 52902 of the MTHFS gene. Examples of SNPs or genetic variants of MTHFS are provided in Tables C and T.

Also provided herein is an isolated nucleic acid corresponding in sequence to an allele of an MAT1A gene, wherein said nucleic comprises a SNP found at a nucleotide selected from the group consisting of nucleotide 5045 of the MAT1A gene; nucleotide 5181 of the MAT1A gene; nucleotide 5233 of the MAT1A gene; nucleotide 6739 of the MAT1A gene; nucleotide 6795 of the MAT1A gene; nucleotide 9833 of the MAT1A gene; nucleotide 10006 of the MAT1A gene; nucleotide 10312 of the MAT1A gene; nucleotide 10339 of the MAT1A gene; nucleotide 10374 of the MAT1A gene; nucleotide 10484 of the MAT1A gene; nucleotide 10555 of the MAT1A gene; nucleotide 14038 of the MAT1A gene; nucleotide 14114 of the MAT1 A gene; nucleotide 14177 of the MAT1A gene; nucleotide 15424 of the MAT1A gene; nucleotide 15500 of the MAT1A gene; nucleotide 15646 of the MAT1A gene; nucleotide 15706 of the MAT1A gene; nucleotide 15715 of the MAT1A gene; nucleotide 15730 of the MAT1A gene; nucleotide 15758 of the MAT1A gene; nucleotide 16133 of the MAT1A gene; nucleotide 16174 of the MAT1A gene; nucleotide 15706 of the MAT1A gene; nucleotide 15715 of the MAT1A gene; nucleotide 15730 of the MAT1A gene; nucleotide 15758 of the MAT1A gene; nucleotide 16133 of the MAT1A gene; nucleotide 16174 of the MAT1A gene; nucleotide 16218 of the MAT1A gene; and nucleotide 16971 of the MAT1A gene. Examples of SNPs or genetic variants of MAT1A are provided in Tables D and O.

Also provided herein is an isolated nucleic acid corresponding in sequence to an allele of an MAT2A gene, wherein said nucleic acid comprises a SNP found at a nucleotide selected from the group consisting of nucleotide 2871 of the MAT2A gene; nucleotide 2873 of the MAT2A gene; nucleotide 2939 of the MAT2A gene; nucleotide 3287 of the MAT2A gene; nucleotide 3394 of the MAT2A gene; nucleotide 3466 of the MAT2A gene; nucleotide 3498 of the MAT2A gene; nucleotide 3650 of the MAT2A gene; nucleotide 3704 of the MAT2A gene; nucleotide 4174 of the MAT2A gene; nucleotide 4449 of the MAT2A gene; nucleotide 4476 of the MAT2A gene; nucleotide 4608 of the MAT2A gene; nucleotide 4660 of the MAT2A gene; nucleotide 4692 of the MAT2A gene; nucleotide 4931 of the MAT2A gene; nucleotide 5313 of the MAT2A gene; nucleotide 5460 of the MAT2A gene; and nucleotide 5480 of the MAT2A gene. Examples of SNPs or genetic variants of MAT2A are provided in Tables E and P.

Also provided herein is an isolated nucleic acid corresponding in sequence to an allele of a GART gene, wherein said nucleic acid comprises a one SNP found at a nucleotide in the GART gene selected from the group consisting of nucleotide 3782 of the GART gene; nucleotide 3842 of the GART gene; nucleotide 7745 of the GART gene; nucleotide 7984 of the GART gene; nucleotide 10775 of the GART gene; nucleotide 11521 of the GART gene; nucleotide 11522 of the GART gene; nucleotide 11541 of the GART gene; nucleotide 12356 of the GART gene; nucleotide 14200 of the GART gene; nucleotide 14273 of the GART gene; nucleotide 14282 of the GART gene; nucleotide 14739 of the GART gene; nucleotide 14781 of the GART gene; nucleotide 18055 of the GART gene; nucleotide 18064 of the GART gene; nucleotide 18130 of the GART gene; nucleotide 18142 of the CART gene; nucleotide 18197 of the GART gene; nucleotide 18232 of the GART gene; nucleotide 18401 of the GART gene; nucleotide 20812 of the CART gene; nucleotide 20825 of the GART gene; nucleotide 16174 of the CART gene; nucleotide 15706 of the CART gene; nucleotide 20862 of the CART gene; nucleotide 22481 of the GART gene; nucleotide 22521 of the CART gene; nucleotide 25425 of the GART gene; nucleotide 25433 of the GART gene; nucleotide 25601 of the GART gene; nucleotide 25867 of the CART gene; nucleotide 25912 of the CART gene; nucleotide 25951 of the CART gene; nucleotide 25956 of the GART gene; nucleotide 26127 of the CART gene; nucleotide 26195 of the CART gene; nucleotide 31627 of the GART gene; nucleotide 31641 of the CART gene; nucleotide 31887 of the CART gene; nucleotide 31902 of the CART gene; nucleotide 31933 of the CART gene; nucleotide 33173 of the CART gene; nucleotide 33264 of the CART gene; nucleotide 31933 of the GART gene; nucleotide 33173 of the GART gene; nucleotide 33264 of the GART gene; nucleotide 33286 of the GART gene; nucleotide 36963 of the GART gene; nucleotide 36964 of the CART gene; nucleotide 37428 of the CART gene; nucleotide 37433 of the GART gene; nucleotide 38762 of the CART gene; nucleotide 38914 of the GART gene; and nucleotide 38989 of the CART gene. Examples of SNPs or genetic variants of GART are provided in Tables F and N.

Also provided herein is an isolated nucleic acid comprising a sequencing in an allele of AHCY, AHCYL1, AHCYL2, ALDH1L1, ALDHL2, AMT, ATIC, BHMT1, BHMT2, CBS, CTH, DHFR, DMGDH, FPGS, GART, GGH, MAT1A, MAT2A, MTFMT, MTHFD1, MTHFD2, MTHFR, MTHFS, MTR, MTRR, NAALAD2, SARDH, SHMT1, SHMT2, or TYMS. The nucleic acid can be a genetic variant, such as a SNP. In some embodiments, the allele comprises a genetic variant of MTHFR, ATIC, MTHFS, MAT1A, MAT2A, GART, AHCY, AMT, CBS, CTH, DHFR, FPGS, MTHFD1, MTHFD2, MTR, SHMT1, SHMT2, or TYMS, such as those listed in Tables A-X. For example, the allele may comprise a genetic variant of AHCY, AMT, CBS, CTH, DHFR, FPGS, MTHFD1, MTHFD2, MTR, SHMT1, SHMT2, or TYMS, such as those listed in Table G, H, J, K, L, M, Q, R, U, V, W, or X. The isolated nucleic acid, or a complement thereof, can comprise a genetic variant or SNP, such as shown in Tables A-X.

Also provided herein are probes, such as from about 10 to about 100, about 20 to about 50, or at least about 10, 15, or 20 nucleotides, to detect a genetic variant of AHCY, AHCYL1, AHCYL2, ALDH1L1, ALDHL2, AMT, ATIC, BHMT1, BHMT2, CBS, CTH, DHFR, DMGDH, FPGS, GART, GGH, MAT1A, MAT2A, MTFMT, MTHFD1, MTHFD2, MTHFR, MTHFS, MTR, MTRR, NAALAD2, SARDH, SHMT1, SHMT2, or TYMS, such as a genetic variant of MTHFR, ATIC, MTHFS, MAT1A, MAT2A, GART, AHCY, AMT, CBS, CTH, DHFR, FPGS, MTHFD1, MTHFD2, MTR, SHMT1, SHMT2, or TYMS, such as those listed in Tables A-X.

In one embodiment, the invention provides isolated nucleic acids corresponding in sequence to human MTHFR alleles comprising a sequence encoding a non-synonymous mutation in the MTHFR protein selected from the group consisting of M110I, H213R, D223N, D291N, R519C, R519L, and Q648P. In one embodiment, the invention provides nucleic acids corresponding in sequence to two or more human MTHFR alleles comprising a sequence encoding a non-synonymous mutation in the MTHFR protein selected from the group consisting of M110I, H213R, D223N, D291N, R519C, R519L, and Q648P.

The term “isolated’ as used herein includes polynucleotides substantially free of other nucleic acids, proteins, lipids, carbohydrates or other materials with which it is naturally associated. Polynucleotide sequences of the invention include DNA and RNA sequences.

The nucleic acids provided herein may be useful as probes (e.g., allele specific oligonucleotide probes) or primers in the methods of detecting disclosed herein. The design of appropriate probes or primers for this purpose requires consideration of a number of factors. For example, fragments having a length of between 10, 15, or 18 nucleotides to about 20, or to about 30 nucleotides, will find particular utility. Longer sequences, e.g., 40, 50, 80, 90, 100, even up to full length, are even more preferred for certain embodiments. Lengths of oligonucleotides of at least about 18 to 20 nucleotides are well accepted by those of skill in the art as sufficient to allow sufficiently specific hybridization so as to be useful as an allele specific oligonucleotide probe. Furthermore, depending on the application envisioned, one will desire to employ varying conditions of hybridization to achieve varying degrees of selectivity of probe towards target sequence. For applications requiring high selectivity, one will typically desire to employ relatively stringent conditions to form the hybrids. For example, relatively low salt and/or high temperature conditions, such as provided by 0.02 M 0.15M NaCl at temperatures of about 50° C. to about 70° C. Such selective conditions may tolerate little, if any, mismatch between the probe and the template or target polynucleotide fragments.

Also provided are vectors comprising nucleic acids of the invention. These vectors include expression vectors that provide for expression of nucleic acids of the invention in appropriate host cells.

Additionally provided are host cells comprising nucleic acids of the invention. Also provided are host cells comprising vectors of the invention. The invention also provides methods of producing enzymes encoded by nucleic acids of the invention, which methods comprise culturing host cells of the invention.

Also provided are isolated enzymes encoded by nucleic acids of the invention.

Detection of Impaired Alleles

The methods disclosed herein (e.g., methods of screening, preventing, and/or treating a condition or disease associated with impaired alleles of genes involved in metabolic pathways) generally require detecting the presence or absence of a plurality of single nucleotide polymorphisms (SNPs) in at least one enzyme-encoding gene within a metabolic pathway that may result in an impaired allele; preferably a plurality of known SNP5 in the test gene. Alleles and/or predetermined sequence SNPs may be detected by allele specific hybridization, a sequence-dependent-based technique which permits discrimination between normal and impaired alleles. An allele specific assay is dependent on the differential ability of mismatched nucleotide sequences (e.g., normal:impaired) to hybridize with each other, as compared with matching (e.g., normal:normal or impaired:impaired) sequences.

A variety of methods are available for detecting the presence of one or more single nucleotide polymorphic in an individual. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA chip technologies such as the Affymetrix SNP chips. These methods may require amplification of the test gene, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the alleles permitted to hybridize to a target molecule obtained from a particular animal or human. If the allele on the target molecule contains a nucleotide that is complementary to the particular exonuclease resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the allele of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of an allele. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site, The method determines the identity of the nucleotide of that site using labeled dideoxynucleotide derivatives, which, if complementary to the nucleotide of the allele will become incorporated onto the terminus of the primer.

An alternative method, known as Genetic Bit Analysis or GBA is described by Goelet, P. et al. (PCT Appln. No. 92/15712). The method of Goelet, P. et al, uses mixtures of labeled terminators and a primer that is complementary to the sequence 3 to an allele. The labeled terminator that is incorporated is thus determined by, and complementary to, the nucleotide present in the allele of the test gene. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying alleles in DNA have been described (Komher, J. S. et al., NucI. Acids. Res. 17:7779-7784 (1989); Sokolov, B. P., NucI. Acids Res. 18:3671 (1990); Syvanen, A.-C., et al., Genomics 8:684-692 (1990); Kuppuswamy, M N et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal, Biochem. 208:171-175 (1993)). These methods differ from GBA™ in that they all rely on the incorporation of labeled deoxynucleotides to discriminate between bases at an allele. In such a format, since the signal is proportional to the number of deoxynucleotides incorporated, single nucleotide polymorphisms that occur in runs of the same nucleotide can result in signals that are proportional to the length of the run (Syvanen, A.-C., et al., Amer. J. Hum. Genet. 52:46-59 (1993)).

Any cell type or tissue may be utilized to obtain nucleic acid samples for use in the diagnostics described herein. In a preferred embodiment, the DNA sample is obtained from a bodily fluid, e.g, blood, obtained by known techniques (e.g. venipuncture) or saliva. Alternatively, nucleic acid tests can be performed on dry samples (e.g. hair or skin). When using RNA or protein, the cells or tissues that may be utilized must express an enzyme-encoding gene.

Detection methods may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT_PCR.

A preferred detection method is allele specific hybridization using probes overlapping a region of at least one allele of an enzyme encoding gene.

Detection of Impaired Alleles Using Allele Specific Hybridization

A variety of methods well-known in the art can be used for detection of impaired alleles by allele specific hybridization. Preferably, the test allele is probed with allele specific oligonucleotides (ASOs); and each ASO comprises the sequence of a known allele. ASO analysis detects specific sequence substitutions in a target polynucleotide fragment by testing the ability of an allele specific oligonucleotide probe to hybridize to the target polynucleotide fragment. Preferably, the allele specific oligonucleotide probe contains the sequence (or its complement) of an impaired allele, The presence of an impaired allele in the target polynucleotide fragment is indicated by hybridization between the allele specific oligonucleotide probe and the target polynucleotide fragment under conditions in which an oligonucleotide probe containing the sequence of a wildtype allele does not hybridize to the target polynucleotide fragment. A lack of hybridization between the allele specific oligonucleotide probe having the sequence of the impaired allele and the target polynucleotide fragment indicates the absence of the impaired allele in the target fragment.

In one embodiment, the test gene(s) may be probed in a standard dot blot format. Each region within the test gene that contains the sequence corresponding to the ASO is individually applied to a solid surface, for example, as an individual dot on a membrane. Each individual region can be produced, for example, as a separate PCR amplification product using methods well-known in the art (see, for example, the experimental embodiment set forth in Mullis, K. B., 1987, U.S. Pat. No. 4,683,202).

Membrane-based formats that can be used as alternatives to the dot blot format for performing ASO analysis include, but are not limited to, reverse dot blot, (multiplex amplification assay), and multiplex allele-specific diagnostic assay (MASDA).

In a reverse dot blot format, oligonucleotide or polynucleotide probes, e.g., having known sequence are immobilized on the solid surface, and are subsequently hybridized with the sample comprising labeled test polynucleotide fragments. In this situation, the primers may be labeled or the NTPs may be labeled prior to amplification to prepare a sample comprising labeled test polynucleotide fragments. Alternatively, the test polynucleotide fragments may be labeled subsequent to isolation and/or synthesis In a multiplex format, individual samples contain multiple target sequences within the test gene, instead of just a single target sequence. For example, multiple PCR products each containing at least one of the ASO target sequences are applied within the same sample dot. Multiple PCR products can be produced simultaneously in a single amplification reaction using the methods of Caskey et al, U.S. Pat. No. 5,582,989. The same blot, therefore, can be probed by each ASO whose corresponding sequence is represented in the sample dots.

A MASDA format expands the level of complexity of the multiplex format by using multiple ASOs to probe each blot (containing dots with multiple target sequences). This procedure is described in detail in U.S. Pat. No. 5,589,330 by A. P. Shuber, and in Michalowsky et al., American Journal of Human Genetics, 59(4): A272, poster 1573 (October 1996), each of which is incorporated herein by reference in its entirety. First, hybridization between the multiple ASO probe and immobilized sample is detected. This method relies on the prediction that the presence of a mutation among the multiple target sequences in a given dot is sufficiently rare that any positive hybridization signal results from a single ASO within the probe mixture hybridizing with the corresponding impaired allele. The hybridizing ASO is then identified by isolating it from the site of hybridization and determining its nucleotide sequence.

Suitable materials that can be used in the dot blot, reverse dot blot, multiplex, and MASDA formats are well-known in the art and include, but are not limited to nylon and nitrocellulose membranes.

When the target sequences are produced by PCR amplification, the starting material can be chromosomal DNA in which case the DNA is directly amplified. Alternatively, the starting material can be mRNA, in which case the mRNA is first reversed transcribed into cDNA and then amplified according to the well known technique of RT-PCR (see, for example, U.S. Pat. No. 5,561,058 by Gelfand et al.)

The methods described above are suitable for moderate screening of a limited number of sequence variations (e.g., impaired alleles). However, with the need in molecular diagnosis for rapid, cost effective large scale screening, technologies have developed that integrate the basic concept of ASO, but far exceed the capacity for mutation detection and sample number. These alternative methods to the ones described above include, but are not limited to, large scale chip array sequence-based techniques. The use of large scale arrays allows for the rapid analysis of many sequence variants. A review of the differences in the application and development of chip arrays is covered by Southern, E. M, Trends In Genetics, 12: 110-115 (March 1996) and Cheng et al, Molecular Diagnosis, 1:183-200 (September 1996). Several approaches exist involving the manufacture of chip arrays. Differences include, but not restricted to: type of solid support to attach the immobilized oligonucleotides, labeling techniques for identification of variants and changes in the sequence-based techniques of the target polynucleotide to the probe.

A promising methodology for large scale analysis on ‘DNA chips’ is described in detail in Hacia et al., Nature Genetics, 14:441447 (1996), which is hereby incorporated by reference in its entirety. As described in Hacia et al., high density arrays of over 96,000 oligonucleotides, each 20 nucleotides in length, are immobilized to a single glass or silicon chip using light directed chemical synthesis. Contingent on the number and design of the allele specific oligonucleotide probe, potentially every base in a sequence can be interrogated for alterations, Allele specific oligonucleotide probes applied to the chip, therefore, can contain sequence variations, e.g., SNPs, that are not yet known to occur in the population, or they can be limited to SNPs that are known to occur in the population.

Prior to hybridization with allele specific oligonucleotide probes on the chip, the test sample is isolated, amplified and labeled (e.g. fluorescent markers) by means well known to those skilled in the art. The test polynucleotide sample is then hybridized to the immobilized allele specific oligonucleotide probes. The intensity of sequence-based techniques of the target polynucleotide fragment to the immobilized allele specific oligonucleotide probe is quantitated and compared to a reference sequence. The resulting genetic information can be used in molecular diagnosis. A common, but not limiting, utility of the ‘DNA chip’ in molecular diagnosis is screening for known SNPs. However, this may impose a limitation to the technique by only looking at mutations that have been described in the field. The present invention allows allele specific hybridization analysis be performed with a far greater number of mutations than previously available. Thus, the efficiency and comprehensiveness of large scale ASO analysis will be broadened, reducing the need for cumbersome end-to-end sequence analysis, not only with known mutations but in a comprehensive manner all mutations which might occur as predicted by the principles accepted, and the cost and time associated with these cumbersome tests will be decreased.

Accordingly, in one aspect, the invention provides methods for detecting impaired alleles of enzyme-encoding genes or enzyme-encoding nucleic acids. For example, provided herein are methods for detecting alleles of MTHFR, ATIC, CBS, CTH, GART, MAT1A, MAT2A, and MTHFS. Also provided herein are methods for detecting alleles of AHCY, AHCYL1, AHCYL2, ALDH1L1, ALDHL2, AMT, BHMT1, BHMT2, DHFR, DMGDH, FPGS, FTCD, GGH, MTFMT, MTHFD1, MTHFD2, MTR, MTRR, NAALAD2, SARDH, SHMT1, SHMT2, or TYMS. Furthermore, the methods can be used to detect genetic variants, such as SNPs, such as those listed in Tables A-X.

In one embodiment, detecting a SNP, or other genetic variant, in an enzyme-encoding nucleic acid involves nucleic acid sequencing. In one embodiment, detecting a mutation in an enzyme-encoding nucleic acid involves PCR. In one embodiment, detecting a mutation in an enzyme-encoding nucleic acid involves RFLP analysis. In one embodiment, detecting a mutation in an enzyme-encoding nucleic acid involves nucleic acid hybridization. Detecting a mutation SNP through hybridization may be done, for example, using a nucleic acid array comprising a nucleic acid that will hybridize under stringent conditions to an enzyme-encoding nucleic acid, or a fragment thereof, comprising such an SNP.

In one embodiment, the methods comprise use of an in vivo assay for determining the activity of an allele of an enzyme-encoding gene, as described herein.

Combinations of methods may also be used to detect and characterize an impaired allele of an enzyme-encoding gene. In one embodiment, the methods comprise use of an in vivo assay for determining the activity of an enzyme-encoding gene, as described herein, and detecting a SNP in an enzyme-encoding nucleic acid.

In one embodiment, the methods comprise use of an in vivo assay for determining enzyme activity, as described herein, and a temperature sensitivity assay to determine enzyme stability at an elevated temperature.

In one embodiment, the methods comprise use of an in vivo assay for determining enzyme activity, as described herein, and an in vitro assay for determining the specific activity of the enzyme.

In a preferred embodiment, an impaired allele of MTHFR comprises a non-synonymous substitution that encodes for a mutation in the MTHFR protein selected from the group consisting of M110I, H213R, D223N, D291N, R519C, R519L, and Q648P. In an especially preferred embodiment, an impaired allele comprises a non-synonymous substitution that encodes for a mutation in the MTHFR protein selected from the group consisting of M110I, H213R, D223N, and D291N.

Yeast Strains

In one aspect, the invention provides yeast strains capable of detecting impaired alleles of enzymes involved in folate/homocysteine metabolism. Such yeast strains are useful in methods disclosed herein. The yeast strains comprise a first mutation allowing for complementation by a functionally homologous enzyme of interest, and a second mutation (or group of mutations) rendering the strain dependent upon supplementation with a cofactor for an assayable phenotype related to function of the first gene.

In one embodiment, the invention provides yeast strains capable of detecting impaired alleles of CTH and determining the responsiveness thereof to vitamin B₆. In a preferred embodiment, the yeast strain comprises a mutation in cys3 and in sextuple-delete sno1Δ sno2Δ sno3Δ snz1Δ snz2Δ snz3Δ.

In one embodiment, the invention provides yeast strains capable of detecting impaired alleles of CBS and determining the responsiveness thereof to vitamin B. In a preferred embodiment, the yeast strain comprises a mutation in cys4 and in sextuple-delete sno1Δ sno2Δ sno3Δ snz1Δ snz2Δ snz3Δ.

In one embodiment, the invention provides yeast strains capable of detecting impaired alleles of MTHFR and determining the responsiveness thereof to folate. In a preferred embodiment, the yeast strain comprises a mutation in met13 and fol3.

Screening for Risk of Disease

In one aspect, the invention provides methods of screening for risk of a condition or disease associated with aberrant folate/homocysteine metabolism. The methods involve screening for an impaired allele of a gene involved in folate/homocysteine metabolism, as described herein.

In one embodiment, the invention provides methods of screening for a risk of a disease or condition associated with an enzyme dysfunction, wherein the enzyme is selected from the group consisting of MTHFR, ATIC, MTHFS, MAT1A, MAT2A, and GART. In a preferred embodiment, the disease or condition is selected from the group consisting of cardiovascular disease, coronary artery disease, ischemic stroke, atherosclerosis, neural tube defects, orofacial clefts, pre-eclampsia, pre term delivery/low birth weight, recurrent early spontaneous abortion, thrombosis, retinal artery occlusion, down's syndrome, colorectal cancer, breast cancer, lung cancer, prostate cancer, depression, schizophrenia, Alzheimer's disease/dementia, age-related macular degeneration, and glaucoma. The methods comprise use of a method for detecting an impaired allele selected from the group consisting of an impaired allele of MTHFR, an impaired allele of ATIC, an impaired allele of MTHFS, an impaired allele of MAT1A, an impaired allele of MAT2A, and an impaired allele of GART, as described herein.

In one embodiment, the invention provides methods of screening for a risk of a disease or condition associated with CBS dysfunction. In a preferred embodiment, the disease or condition is selected from the group consisting of cardiovascular disease, coronary artery disease, ischemic stroke, atherosclerosis, neural tube defects, orofacial clefts, pre-eclampsia, pre-term delivery/low birth weight, recurrent early spontaneous abortion, thrombosis, retinal artery occlusion, down's syndrome, colorectal cancer, breast cancer, lung cancer, prostate cancer, depression, schizophrenia, Alzheimer's disease, dementia, age-related macular degeneration, and glaucoma. The methods comprise use of a method for detecting an impaired CBS allele, as described herein.

In one embodiment, the invention provides methods of screening for a risk of a disease or condition associated with CTH dysfunction. In a preferred embodiment, the disease or condition is selected from the group consisting of cardiovascular disease, coronary artery disease, ischemic stroke, atherosclerosis, neural tube defects, orofacial clefts, pre-eclampsia, pre-term delivery/low birth weight, recurrent early spontaneous abortion, thrombosis, retinal artery occlusion, down's syndrome, colorectal cancer, breast cancer, lung cancer, prostate cancer, depression, schizophrenia, Alzheimer's disease/dementia, age-related macular degeneration, and glaucoma. The methods comprise use of a method for detecting an impaired CTH allele, as described herein.

Screening for Chemotherapeutic Response Potential

In one aspect, the invention provides methods of determining an individual's chemotherapeutic response potential. The methods comprise use of a method for detecting an impaired allele of a gene involved in folate/homocysteine metabolism, as described herein. In a preferred embodiment, the gene is selected from the group consisting of MTHFR, ATIC, MTHFS, MAT1A, MAT2A, and GART. Detection of an impaired allele in an individual indicates a decreased response potential.

In a preferred embodiment, the chemotherapeutic is methotrexate or 5-fluorouracil.

Screening for Chemotherapeutic Toxicity

In one aspect, the invention provides methods of determining chemotherapeutic toxicity for an individual. The methods comprise use of a method for detecting an impaired allele of a gene involved in folate/homocysteine metabolism, as described herein. In a preferred embodiment, the gene is selected from the group consisting of MTHFR, ATIC, MTHFS, MAT1A, MAT2A, and GART. Detection of an impaired allele in an individual indicates an increased toxicity potential.

In a preferred embodiment, the chemotherapeutic is methotrexate or 5-fluorouracil.

Prophylaxis and Treatment

In one aspect, the invention provides methods of preventing a condition or disease associated with metabolic enzyme deficiency. The methods comprise increasing an individual's intake of a cofactor based on information obtained from the foregoing assays and methods, which inform on the presence of cofactor-sensitive impaired alleles. In a preferred embodiment, the methods comprise detecting a cofactor-remediable impaired allele of a metabolic gene, as described herein.

In one embodiment, the invention provides methods of preventing a condition or disease associated with aberrant folate/homocysteine metabolism. The methods comprise increasing an individual's intake of folate and/or vitamin B. In a preferred embodiment, the methods comprise detecting an impaired allele of a gene involved in folate/homocysteine metabolism, as described herein.

In one embodiment, the invention provides a method of preventing a condition or disease associated enzyme dysfunction in an individual having an impaired allele of an enzyme-encoding gene that is cofactor remediable, wherein the enzyme-encoding gene is selected from the group consisting of MTHFR, ATIC, MTHFS, MAT1A, MAT2A, and GART. The method comprises increasing the individual's intake of folate.

In one embodiment, the invention provides a method of preventing a condition or disease associated CBS dysfunction in an individual having an impaired CBS allele. The method comprises increasing the individual's intake of vitamin B₆.

In one embodiment, the invention provides a method of preventing a condition or disease associated CTH dysfunction in an individual having an impaired CTH allele. The method comprises increasing the individual's intake of vitamin B₅.

In one aspect, the invention provides methods of treating a condition or disease associated with aberrant folate/homocysteine metabolism. The methods comprise increasing an individual's intake of folate and/or vitamin B₆. In a preferred embodiment, the methods comprise detecting an impaired allele of a gene involved in folate/homocysteine metabolism, as described herein.

In one embodiment, the invention provides a method of treating a condition or disease associated with enzyme dysfunction in an individual having an impaired allele of an enzyme-encoding gene that is co-factor remediable, wherein the enzyme-encoding gene is selected from the group consisting of MTHFR, ATIC, MTHFS, MAT1A, MAT2A, and GART remediable by cofactor, wherein the. The method comprises increasing the individual's intake of folate.

In one embodiment, the invention provides a method of treating a condition or disease associated CBS dysfunction in an individual having an impaired CBS allele. The method comprises increasing the individual's intake of vitamin B₆.

In one embodiment, the invention provides a method of treating a condition or disease associated CTH dysfunction in an individual having an impaired CTH allele. The method comprises increasing the individual's intake of vitamin B₆.

### Formulations

The present invention further provides a formulation comprising one or more cofactors for an individual. The one or more cofactors are selected based on the genetic makeup of the individual. For example, the formulation can comprise a plurality of cofactors, wherein at least a subset of the cofactors within the plurality of cofactors is selected based on the genetic makeup of an individual. In some embodiments, all of the cofactors selected to be in the formulation are based on the genetic makeup of the individual. For example, a subset, such as at least 1 of the plurality cofactors present in a formulation can be based on the genetic makeup of the individual. In other embodiments, at least 2 or more of the plurality of cofactors present in the formulation is based on the individual's genetic makeup. In yet other embodiments, at least 3, 4, 5, 6, 7, 8, 9, 10, 11, 12, 13, 14, 15, 16, 17. 18, 19, 20, 21, 22, 23, 24, 25, 50, 75, or 100 cofactors are present in the formulation, wherein all, or a subset of the cofactors present in the formulation, is selected for the formulation based on the genetic-makeup of the individual.

The formulation disclosed herein can comprise one or more cofactors that are present in an amount determined by the genetic makeup of an individual. In one embodiment, the formulation comprises a cofactor, wherein the cofactor is present in an amount selected based on genetic makeup of an individual. In another embodiment, the formulation comprises a plurality of cofactors in which at least a subset of the cofactors is present in an amount based on the genetic makeup of the individual. In other embodiments, at least 2 or more of the plurality of cofactors present in the formulation is present in an amount based on the individual's genetic makeup. In yet other embodiments, at least 3, 4, 5, 6, 7, 8, 9, 10, 11, 12, 13, 14, 15, 16, 17. 18, 19, 20, 21, 22, 23, 24, 25, 50, 75, or 100 cofactors are present in the formulation, wherein all, or a subset of the cofactors present in the formulation, is present in an amount based on the genetic makeup of the individual.

### Analysis of Genetic Makeup

The formulations disclosed herein comprise one or more cofactors selected based on the genetic makeup of an individual. The genetic makeup of an individual can be determined through analysis of a biological sample of the individual. Analysis can comprise detecting the absence or presence of a genetic variant correlated with a cofactor enzyme deficiency or cofactor remediable condition. In some embodiments, a plurality of genetic variants is analyzed. The presence or absence of one or more genetic variants can be used to determine the risk or predisposition the individual has of a cofactor-dependent enzyme deficiency. The presence or absence of one or more genetic variants can be used to determine the risk or predisposition the individual has of a cofactor remediable condition.

The genetic makeup of an individual can be obtained through analysis of a biological sample of an individual. The individual can provide a biological sample, such as any sample from which a genetic sample may be derived. Samples may be from buccal swabs, saliva, blood, hair, or any other type of sample obtained from the individual. The sample can be obtained by a third party and analyzed by another party. The sample may have been previously stored. Alternatively, the sample can be obtained and analyzed by a single party.

The individual can be an animal, such as a mouse, rat, rabbit, cat, dog, horse, chicken, sheep, cow, monkey or other animal. In some embodiments, the individual is a human. The human can be of any age. The human can be a fetus, baby, child, adolescent, adult, or geriatric individual. The individual may be an adult over 50 years of age, over 60 years of age, or more. The individual may be of child-bearing age. The individual may be a female or male.

In some embodiments, the individual is a pregnant female. In yet other embodiments, the individual may be the parent, female or male, of a soon to be born child, such as a fetus, or as yet to be conceived child. For example, the genetic makeup of a female, such as a female interested in having children or a pregnant female, is analyzed to determine the risk of the child having a condition, such as a condition dependent on an enzyme deficiency or is cofactor remediable. In yet other embodiments, the genetic makeup of the father or father to be of the child is analyzed. Formulations for the mother and father can be determined based on their genetic makeup, wherein the formulation can improve the health of the mother, father, and/or child, remedy a cofactor dependent enzyme deficiency of the mother, father, and/or child, or remedy a cofactor remediable condition of the mother, father and/or child.

The individual may have a family history of metabolic conditions, such as a cofactor-dependent enzyme deficiency. In some embodiments, the individual does not experience any symptoms or conditions of a metabolic condition, such as a cofactor-dependent enzyme deficiency. In yet other embodiments, the individual is experience one or more symptoms or conditions of a metabolic condition, such as a cofactor-dependent enzyme deficiency.

The genetic makeup of an individual can be analyzed to determine the predisposition, risk, diagnosis, prognosis, or theranosis of a metabolic condition, such as a cofactor dependent enzyme deficiency. The analysis can be used to determine the presence or absence, an effectiveness of a treatment, or a response to a treatment of a cofactor dependent enzyme deficiency. In some embodiments, the analysis can be used to determine the presence or absence, an effectiveness of a treatment, or a response to a treatment of a cofactor remediable condition.

For example, the cofactor remediable condition can be a vitamin deficiency, or exhibits symptoms of such a deficiency. The vitamin deficiency can be a vitamin A deficiency, hypervitaminosis A, vitamin D deficiency and dependency, hypervitaminosis D, vitamin E deficiency and toxicity, vitamin K deficiency, hypervitaminosis K, essential fatty acid deficiency, thiamine deficiency, riboflavin deficiency, niacin deficiency, vitamin B.sub.6 deficiency and dependency, biotin deficiency and dependency, pantothenic acid deficiency, carnitine deficiency or vitamin C deficiency. In another embodiment, the cofactor remediable condition can be a mineral deficiency, or exhibits symptoms of such a deficiency. For example, the mineral deficiencies can include phosphate depletion, iodine deficiency, fluorine deficiency, zinc deficiency disturbances in copper metabolism, acquired copper deficiency, acquired copper toxicosis, inherited copper deficiency or inherited copper toxicosis.

The cofactor remediable condition may be an avitiminoses or hypervitaminosis. In some embodiments, the aviatmines, without being bound by theory, is a vitamin A deficiency, resulting in conditions such as xerophthalmia or night blindness; a thiamine deficiency, resulting in conditions such as beriberi; a niacin deficiency, resulting in conditions such as pellagra; a vitamin B12 deficiency, resulting in conditions such as megaloblastic anemia; a vitamin C deficiency, resulting in conditions such as scurvy; a vitamin D deficiency, resulting in conditions such as rickets, or a vitamin K deficiency resulting in conditions such as impaired coagulation.

The cofactor remediable condition can also include conditions such as immune conditions, child development, cardiovascular conditions, and effects of aging. For example, the cofactor remediable condition can be low bone density, Cohn's disease, or multiple sclerosis.

In some embodiments, the cofactor remediable condition includes having a preterm birth. Other conditions include having an offspring with spina bifida, disorders in growth and mental development, cleft palate, anencephaly, or any other neural tube defects (NTDs). In some embodiments, a cofactor remediable condition is the ability or predisposition to have a child with a cofactor remediable condition. In some embodiments, a cofactor remediable condition is the risk or predisposition of having a child with birth defects, such as neural tube defects. In some embodiments, the NTD is spina bifida. Other defects can include preterm birth or cleft palate.

In other embodiments, the genetic makeup of an individual is analyzed and the individual has a low risk or predisposition to a cofactor remediable condition. The analysis can be used to provide information for selecting a cofactor, or a plurality of cofactors, for a formulation for the individual that improves the individual's health. Alternatively, the formulation can aid in the amelioration of one or more symptoms of a known or unknown condition of the individual, such as a cofactor dependent enzyme deficiency.

The genetic makeup of an individual can also provide information on the amount of one cofactor, or a plurality of cofactors, present in a formulation for the individual. For example, if the genetic makeup of an individual is analyzed and the individual has a low risk or predisposition to a cofactor remediable condition, the formulation can comprise lower amounts of one or more cofactors, as compared to the recommended dosage amounts or daily intake amounts based as indicated in guidelines (see for example Table 5 and 6). Alternatively, the formulation can comprise higher amounts than recommended dosage amounts or daily intake amounts, for an individual with a risk or diagnosis of a cofactor dependent enzyme deficiency or cofactor remediable condition.

Analysis of one or more genetic variants can be used to determine a risk or predisposition or diagnosis of one or more cofactor remediable condition. For example, the presence or absence of a plurality of genetic variants from a biological sample of an individual can indicate that the individual is at risk of a cofactor-dependent enzyme deficiency. The cofactor-dependent enzyme deficiency can be a cofactor remediable condition. The plurality of genetic variants may comprise at least 2, 3, 4, 5, 6, 7, 8, 9, 10, 15, 20, 25, 30, 40, 50, 75, or 100 genetic variants. The genetic variants can be of the same gene, of different genes in the same metabolic pathway, or of different genes in different metabolic pathway. The analysis of a plurality of genetic variants can provide a more comprehensive or specific formulation for an individual that provides improved health benefits or improved amelioration of one or more symptoms of a cofactor remediable condition as compared to a formulation based on an analysis of a single genetic variant or less than the plurality of genetic variants.

The genetic variant can be a single nucleotide polymorphism (SNP), truncation, insertion, deletion, or repeat. The genetic variant can also be a nucleotide repeat, nucleotide insertion, nucleotide deletion, chromosomal translocation, chromosomal duplication, or copy number variation. In some embodiments, the copy number variation is a microsatellite repeat, nucleotide repeat, centromeric repeat, or telomeric repeat.

The genetic variant can be of a gene in a metabolic pathway such as a pathway for the biosynthesis of a cofactor, such as a vitamin. For example, the pathway may include, but not be limited to, thiamine metabolic pathway, riboflavin metabolic pathway, vitamin B6 metabolic pathway, nicotinate and nicotinamide metabolic pathway, pantothenate and CoA biosynthesis pathway, biotin metabolic pathway, lipoic metabolic pathway, folate/homocysteine metabolic pathway, retinol metabolic pathway, porphyrin metabolic pathway, ubiquinone and other terpenoid-quinone biosynthesis pathway.

The genetic variant can be of a gene in the pathway for metabolizing Vitamin A (retinol), Vitamin C (ascorbic acid), Vitamin D (calciferol), Vitamin E, Vitamin K (phylloquinone), Vitamin B1 (Thiamin), Vitamin B2 (riboflavin), Vitamin B3 (niacin), Vitamin B6 (pyridoxine), Vitamin B9 (folate/folic acid), Vitamin B12 (tocopherol), Vitamin B7 (biotin), Vitamin B5 (panthothenic acid), or choline.

In one embodiment, the genetic variant is of a gene in the folate pathway, such as AHCY, AHCYL1, AHCYL2, ALDH1L1, ALDHL2, AMT, ATIC, BHMT1, BHMT2, CBS, CTH, DHFR, DMGDH, FPGS, FTCD, GART, GGH, MAT1A, MAT2A, MTFMT, MTHFD1, MTHFD2, MTHFR, MTHFS, MTR, MTRR, NAALAD2, SARDH, SHMT1, SHMT2, or TYMS. The genetic variant, such as a SNP, can be selected a genetic variant of MTHFR, ATIC, MTHFS, MAT1A, MAT2A, GART, AHCY, AMT, CBS, CTH, DHFR, FPGS, MTHFD1, MTHFD2, MTR, SHMT1, SHMT2, or TYMS. For example, the genetic variant can be selected from Tables A-X. In some embodiments, the genetic variant is of AHCY, AMT, CBS, CTH, DHFR, FPGS, MTHFD1, MTHFD2, MTR, SHMT1, SHMT2, or TYMS, such as one or more listed in Tables G, H, J, K, L, M, Q, R, U, V, W, and X.

The genetic variants may be identified through published literature or scientific journals or meetings. Alternatively, they may be genetic variants identified through the methods disclosed herein. For example, one or more individuals may have a known metabolic condition such as a cofactor remediable condition. Their genomes may be analyzed and genetic variants identified and the genetic variants can be used in the methods disclosed herein, such as detecting the genetic variant in other individuals and identifying the cofactor remediable condition in other individuals. Thus another aspect of the present disclosure is the updating the list of genetic variants with genetic variants as well as genetic variants identified through scientific literature and other publicly available sources. For example, an existing database of genetic variants and their correlations to cofactor dependent enzyme deficiencies, cofactor remediable conditions, recommended cofactors and amounts of the cofactors can be updated with the genetic variants or new genetic variants identified through publicly available sources.

A genetic variant can be analyzed using any method known in the arts, such as those described herein. For example, analysis can be performed by DNA sequencing, PCR based methods such as real-time PCR, mass spectrometry (MALDI-TOF/MS method), bead-based assays, melting curve analysis, or microarrays. Analysis can also be performed by fragment length polymorphism assays (restriction fragment length polymorphism (RFLP), cleavage fragment length polymorphism (CFLP)), single-strand conformation polymorphism analysis hybridization methods using an allele-specific oligonucleotide as a template (e.g., TaqMan PCR method, the invader method, the DNA chip method), primer extension reaction methods, Amplification Refractory Mutation System (ARMS) and the like can also be used.

Any commercially available kits, systems, and platforms can be used for analyzing the genetic variants described herein. For example, arrays, such as, but not limited to, arrays from Affymetrix (Santa Clara, Calif.) such as the Affymetrix Genome-Wide Human SNP Array 6.0, or Agilent (Santa Clara, Calif.), such as the Human Genome CGH Microarray Kit 244A, and related products can be used. Bead-based platforms, such as from Illumina (San Diego, Calif.), such as Infinium HD BeadChips or Genome Analzyer, and related platforms and technologies can also be used. Sequencing platforms commercially available or under development, such as from Illumina, Applied Biosystems (Foster City, Calif.), such as the Genetic Analyzer; 454 Life Sciences (Branford, Conn.), such as the Genome Sequencer; Helicos BioSciences Corporation (Cambridge, Mass.), such as the Helicos™ Genetic Analysis System; and other related products or technologies can also be used. Other platforms, such as use of melting-curve analysis, such as, but not limited to, the use of Qiagen HRM PCR kit, Catalog No. 6569627) and PCR-based methods, such as, but not limited to, the use of TaqMan® PCR (such as from Roche, Base Switzerland), or quantitative real-time ARMS, such as through the use of Scorpion® Primers (DxS Ltd, Manchester, UK), can also be used.

Detection of the genetic variant can be indirect or direct. For example, a genetic variant correlated with a cofactor remediable condition, “SNP A” can be directly detected. Alternatively, SNP A can be in linkage disequilibrium with another genetic variant, “SNP B”. As such, SNP A can be indirectly detected through detection of SNP B.

In some embodiments, microarrays are used to detect the genetic variants. For example, a microarray can comprise one or more nucleic acids to detect the one or more genetic variants in a sample. The microarray can comprise nucleic acids to detect one or more genetic variants such as those listed in Tables A-X. For example, the microarray can comprise immobilized thereon, a plurality of isolated nucleic acids comprising genetic variants such as those listed in Tables A-X. The microarray can comprise probes for specifically detecting one or more genetic variants such as those listed in Tables A-X.

### Cofactors

The formulations disclosed herein can include one or more cofactors, such as a plurality of cofactors, wherein a subset of the plurality or all of the cofactors, in the formulation is selected from the genetic makeup of the individual. Furthermore, the amount of the one or more cofactors in the formulation can also be determined from the genetic makeup of the individual.

A cofactor is a non-protein compound, naturally occurring or synthetic, that associates with a protein and aids the protein's biological activity. For example, cofactors commonly associate with enzymes and are often times required for the enzyme's activity. The cofactor may be loosely bound or associated with a protein and termed a coenzyme. In other embodiments, the cofactor is tightly associated or bound to a protein, such as a prosthetic group. A cofactor disclosed herein can be a direct cofactor of an enzyme of interest (e.g., folate for MTHFR, ATIC, GART, MAT1A, MAT2A, and MTHFS), as well as an indirect cofactor for an enzyme of interest. Thus, cofactors can directly or indirectly impact enzyme function.

The cofactors disclosed herein can be synthetic or naturally occurring. For example, the cofactors can be manufactured through chemical synthesis, in vitro, or purified from organisms. The formulations can comprise cofactors that are synthetic, naturally occurring, or a combination thereof.

The cofactors can be organic or inorganic. The formulations disclosed herein can comprise one or more cofactors that are organic, inorganic, or a combination thereof. For example, the cofactor can be an organic cofactor, such as a vitamin or a molecule derived from a vitamin. The cofactor may contain the nucleotide adenosine monophosphate (AMP) as part of their structure, such as ATP, coenzyme A, FAD and NAD⁺. Other organic cofactors include flavin or heme.

The cofactor selected based on the genetic makeup of an individual can be a vitamin, such as those in Table 5. The vitamin can be water-soluble or fat-soluble. For example, the formulation can comprise one or more of the following vitamins: Vitamin A (retinol), Vitamin C (ascorbic acid), Vitamin D (calciferol), Vitamin E, Vitamin K (phylloquinone), Vitamin B1 (Thiamin), Vitamin B2 (riboflavin), Vitamin B3 (niacin), Vitamin B6 (pyridoxine), Vitamin B9 (folate/folic acid), Vitamin B12 (tocopherol), Vitamin B7 (biotin), Vitamin B5 (panthothenic acid), or choline. The formulation can comprise at least 1, 2, 3, 4, 5, 6, 7, 8, 9, 10, 11, 12, 13, or 14 of the aforementioned vitamins. In some embodiments, the formulation does not comprise a vitamin.

The cofactor can also be an inorganic cofactor, such as a mineral or metal ion, such as those listed in Table 6. For example, the inorganic cofactor can be metal ions such as, but not limited to, Mg²⁺, Cu⁺, Mn²⁺ or iron-sulfur clusters. The formulations disclosed herein can comprise any one or more of calcium, phosphorus, iron, iodine, magnesium, zinc, selenium, copper, manganese, chromium, or molybdenum. The formulation can comprise at least 1, 2, 3, 4, 5, 6, 7, 8, 9, 10, or 11, of the aforementioned minerals. In some embodiments, the formulation does not comprise a mineral.

### Amount of Cofactors

In another aspect of the formulations disclosed herein, the amount of one or more cofactors in the formulation is selected based on the genetic makeup of the individual. In one embodiment, the formulation comprises a cofactor in which the amount of the cofactor is determined by the genetic makeup of the individual. In another embodiment, the formulation comprises a plurality of cofactors in which a subset of the cofactors, or all of the cofactors, is present in an amount determined by the genetic makeup of the individual. The amount recommended in a formulation is typically based on the dosing regimen of the formulation, for example, the amount may be based on a daily intake of the formulation. Alternatively, the amount may be based on a twice daily, thrice daily, weekly, biweekly, monthly or bimonthly regimen.

The genetic makeup of an individual can be used to determine that the amount of one or more cofactors that the individual should take or be recommended to take, or supplement their diet with, is different than that recommended for another individual with a different genetic makeup. For example, the presence or absence of at least one genetic variant that correlates to a need for supplementing a cofactor in an individual's diet can be detected in a biological sample of an individual. Detecting the presence of the genetic variant can then be used to determine that the recommended amount of a cofactor for the individual is different than the amount recommended for an individual lacking the genetic variant. Alternatively, detecting the absence of a genetic variant can also be used to determine that the recommended amount of a cofactor for the individual is different than the amount recommended for an individual with the genetic variant. In some embodiments, the absence or presence of a plurality of genetic variants is used to determine that the amount of one or more cofactors that the individual should take, or supplement to their diet. For example, the presence or absence of least 2, 3, 4, 5, 6, 7, 8, 9, 10, 15, 20, 25, 30, 40, 50, 75, or 100 genetic variants can be detected in an individual and correlated to the amount of one or more cofactors an individual should take, or supplement their diet with.

The difference in a recommended amount of a cofactor between an individual with a particular genetic variant as compared to an individual without the particular genetic variant can be a difference of at least about 1, 5, 10, 15, 20, 25, 30, 35, 40, 45, 50, 75, 100, 125, 150, 175, 200, 250, 300, 350, 400, 450, 500, 600, 700, 800, 900, 1000, 1500, 2000, 3000, 4000, or 5000% of the weight, mass, or IU of the cofactor. The weight can be the dry weight of the cofactor or the equivalent weight or biological activity of the cofactor.

For example, an individual with a particular SNP may have a recommended daily dose of 400 mcg of folic acid; however, an individual without that SNP may have a recommended daily dose that is 1000% of that, which is 4 mg of folic acid. In another example, an individual with a particular SNP may have a recommended daily dose of 400 mcg of folic acid; however, an individual without that SNP may have a recommended daily dose that is 25% of that, which is 100 mcg of folic acid

In some embodiments, the presence of a genetic variant in an individual is correlated to a recommendation that the individual intake an amount of cofactor that is greater than the amount of a cofactor recommended for an individual without the genetic variant. Alternatively, the absence of a genetic variant in an individual is correlated to a recommendation that the individual intake an amount of cofactor that is greater than the amount of a cofactor recommended for an individual with the genetic variant. For example, the presence of the genetic variant can be used to determine that the individual should intake an amount of cofactor that is at least about 1.1 times greater than the amount an individual without the genetic variant should take or supplement their diet with. In other embodiments, the amount of cofactor the individual with the genetic variant should take is at least about 1.2, 1.3, 1.4, 1.5, 2, 3, 4, 5, 6, 7, 8, 9, 10, 20, 30, 40, or 50 times the amount an individual without the genetic variant is recommended to take. For example, the presence of a genetic variant is detected in a sample from a pregnant woman. The presence of the genetic variant is correlated with a recommendation that the individual supplement her diet with 5 times the amount of a cofactor, such as folic acid, as compared to an individual without the genetic variant, which reduces the risk of a cofactor-dependent enzyme deficiency, such as preterm birth or birth of a child with spina bifida or cleft palate.

In yet other embodiments, detecting the presence of a genetic variant is correlated to the individual being recommended to intake an amount of a cofactor that is less than the amount an individual without the genetic variant is recommended to take. Alternatively, detecting the absence of a genetic variant is correlated to the individual being recommended to intake an amount of a cofactor that is less than the amount an individual with the genetic variant is recommended to take For example, the presence of a genetic variant in an individual's sample can indicate that the individual should take about 1.1, 1.2, 1.3, 1.4, 1.5, 2, 3, 4, 5, 6, 7, 8, 9, 10, 20, 30, 40, or 50 times less than the amount of the cofactor as recommended for an individual without the genetic variant.

The genetic makeup of an individual can also be used to determine that the amount of one or more cofactors the individual should take, or supplement their diet with, is greater than, less than, or equal to an amount recommended by a government agency or health organization. For example, the genetic makeup of an individual can be used to determine that the amount of one or more cofactors to be taken by the individual can be more than, less than, or equal to that recommended by the Food and Drug Administration (FDA).

The genetic makeup of an individual can be used to determine that one or more cofactors taken by an individual should be greater than, less than or equal to the Reference Daily Intake (RDI) (see for example Tables 5 and 6). For example, women of childbearing age are recommended to obtain 400 mcg of synthetic folic acid (see for example Table 5). However, analysis of the woman's genetic makeup can determine that the woman should obtain at least 5 times or at least 10 times the amount, such as at least 4 mg of folic acid. In another embodiment, an individual's genetic makeup may be used to determine that an individual should have a daily intake of a cofactor that is less than that recommended in the RDI. For example, a formulation of an individual may comprise an amount of folic acid that is half the amount of the RDI.

An individual's sample can be analyzed for the presence or absence of at least one genetic variant that correlates to an amount of cofactor that should be supplemented into the individual's diet. For example, detecting the presence of the genetic variant can then be used to determine that the recommended amount of a cofactor for the individual is different than the amount recommended by a health organization. Alternatively, detecting the absence of a genetic variant can also be used to determine that the recommended amount of a cofactor for the individual is different than the amount recommended by a government agency, such as the RDI amount. In some embodiments, the absence or presence of a plurality of genetic variants is used to determine that the amount of one or more cofactors that the individual should take, or supplement to their diet. For example, the presence or absence of least 2, 3, 4, 5, 6, 7, 8, 9, 10, 15, 20, 25, 30, 40, 50, 75, or 100 genetic variants can be detected in an individual and correlated to the amount of one or more cofactors an individual should take, or supplement their diet with.

The difference in a recommended amount of a cofactor between an individual with a particular genetic variant as compared to a recommended value suggested by a government agency, such as the RDI, can be a difference of at least about 1, 5, 10, 15, 20, 25, 30, 35, 40, 45, 50, 75, 100, 125, 150, 175, 200, 250, 300, 350, 400, 450, 500, 600, 700, 800, 900, 1000, 1500, 2000, 3000, 4000, or 5000% of the weight, mass, or IU of the cofactor. For example the RDI for folic acid is 400 mcg; however, an individual with a particular SNP may have a recommended daily dose that is 25% of that, which is 100 mcg of folic acid. In another example, an individual with a particular SNP may have a recommended daily dose of 1000% of the RDI, which is 4 mg of folic acid

In some embodiments, the presence of a genetic variant in an individual is correlated to a recommendation that the individual intake an amount of cofactor that is greater than the amount of a cofactor recommended by a government agency, such as the RDI amount. Alternatively, the absence of a genetic variant in an individual is correlated to a recommendation that the individual intake an amount of cofactor that is greater than the amount of a cofactor recommended by a government agency or health organization. For example, the presence of the genetic variant can be used to determine that the individual should intake an amount of cofactor that is at least about 1.1 times greater than the amount recommended by a government agency or health organization. In other embodiments, the amount of cofactor the individual with the genetic variant should take is at least about 1.2, 1.3, 1.4, 1.5, 2, 3, 4, 5, 6, 7, 8, 9, 10, 20, 30, 40, or 50 times the amount recommended by a government agency or health organization. For example, the presence of a genetic variant is detected in a sample from a pregnant woman. The presence of the genetic variant is correlated with a recommendation that the individual supplement her diet with 5 times the amount of a cofactor, such as folic acid, as compared to the RDI amount, which aids in reducing the risk of a cofactor-dependent enzyme deficiency, such as preterm birth or birth of a child with spina bifida or cleft palate.

In yet other embodiments, detecting the presence of a genetic variant is correlated to the individual being recommended to intake an amount of a cofactor that is less than the amount recommended by a government agency or health organization. Alternatively, detecting the absence of a genetic variant is correlated to the individual being recommended to intake an amount of a cofactor that is less than the amount recommended by a government agency or health organization. For example, the presence of a genetic variant in an individual's sample can indicate that the individual should take about 1.1, 1.2, 1.3, 1.4, 1.5, 2, 3, 4, 5, 6, 7, 8, 9, 10, 20, 30, 40, or 50 times less than the amount of the cofactor as recommended for an individual without the genetic variant.

### Personal Characteristics

The selection of one or more cofactors, the amount of the one or more cofactors, or both, for a formulation for an individual can also be based on the personal characteristic of the individual. For example, the selection of one or more cofactors can be based on the genetic makeup of an individual and one or more personal characteristics of the individual. The personal characteristic can be, but not be limited to, one or more of the following: the weight, height, body-mass index, ethnicity, ancestry, gender, age, family history, medical history, exercise habits, or dietary habit of said individual.

For example, analysis of an individual's genetic makeup is used to determine a plurality of cofactors for an individual. The cofactors include 2 vitamins and a mineral. The individual's dietary habits indicate the individual has a high intake of the mineral, thus the recommended formulation for the individual comprises the 2 vitamins.

In another example, analysis of an individual's genetic makeup is used to determine a plurality of cofactors for an individual. The cofactors include 3 vitamins of varying amounts. The individual's dietary habits indicates the individual has a low intake of these vitamins, thus the formulation comprising the 3 vitamins are in dosages higher than analysis of the genetic makeup of the individual alone would have indicated. Alternatively, the individual's diet may indicate a high intake of these vitamins and accordingly, the formulation of the vitamins contains a decreased amount of the vitamins as compared to the amounts as determined by analysis of the genetic makeup of the individual alone.

In one embodiment, the individual's characteristic is gender and pregnancy state, such that a genetic analysis alone would determine that a female should take 600 mcg of folic acid. However, taking into account her personal characteristic of being pregnant, the recommended amount of folic acid is 4 mg.

In yet other embodiments, characteristics such as an individual's metabolic rate, expression levels of proteins, nucleic acids (such as mRNA, miRNA), levels of metabolites, may also be incorporated into the selection of the one or more cofactors, the amount of one or more cofactors, or both, of a formulation for an individual.

The term “individual” or “subject” is used interchangeably, and refers to a mammalian subject including human subject.

### Dosage Forms

The formulation comprising one or more cofactors selected by the genetic makeup of an individual can be prepared by any means known in the arts. The desirable dose, such as the amount of the one or more cofactors as determined by the genetic makeup of an individual, can vary depending also on the personal characteristics of the individual, such as weight of the subject, as well as the drug form, route and period of administration. For example, the formulation can be formulated for oral, rectal, parenteral, enteral, transdermal, intravenous, topical, subcutaneous, intramuscular or feeding tube administration.

Also provided herein is a method of preparing a formulation can comprise selecting a cofactor, wherein the cofactor is present in an amount selected based on genetic makeup of an individual; and mixing the cofactor with an excipient in an ingestible or injectable form. The method of preparing a formulation can comprise selecting a plurality of cofactors, wherein at least a subset of the cofactors is selected based on genetic makeup of an individual; and mixing the cofactor with an excipient in an ingestible or injectable form.

The formulation can be prepared as a sustained release form or a quick release form. The formulation can be prepared as a unit dosage. In some embodiments, the formulation is orally ingestible. The formulation can be in a powder form, or can be in the form of a granule, tablet or capsule. The formulation can also be in liquid form.

The formulation can comprise one or more cofactors selected by the genetic makeup of the individual and compounded, for example, with the usual non-toxic pharmaceutically acceptable carriers for tablets, pellets, capsules, suppositories, solutions, emulsions, suspensions, and any other form suitable for use. Formulations can also comprise carriers such as talc, water, glucose, lactose, gum acacia, gelatin, mannitol, starch paste, magnesium trisilicate, corn starch, keratin, colloidal silica, potato starch, urea and other carriers suitable for use in manufacturing preparations, in solid, semisolid or liquid form and in addition auxiliary, stabilizing, thickening and coloring agents and perfumes may be used.

For preparing solid compositions of the formulations disclosed herein, such as a tablet form, such as caplets, capsules, including soft gelatin capsules, and lozenges. The solid form of a formulation disclosed herein can be made by methods known in the art and may further comprise suitable binders, lubricants, diluents, disintegrating agents, colorants, flavoring agents, flow-inducing agents, melting agents, many varieties of which are known in the art. The oral dosage forms of the present invention may, optionally, have a film coating to protect the formulation from one or more of moisture, oxygen and light or to mask any undesirable taste or appearance. Suitable coating agents include, for example, cellulose, hydroxypropylmethyl cellulose.

In some embodiments, the formulation of the one or more cofactors is a plurality of beads encapsulated in a capsule. For example, in a plurality of beads, various subsets of the beads can comprise various cofactors. Alternatively the plurality of beads can be of a single cofactor. In some embodiments, each bead can have a diameter from about 1 μm to about 1000 μm and contains a cofactor. In some embodiments, the size ranges from about 300 μm to about 900 μm or from about 450 μm to about 825 μm. Each bead can have the same cofactor or different cofactor. In some embodiments, a plurality of beads in a capsule comprises different cofactors are present in different subsets of the plurality of beads.

In some embodiments, the bead may comprise a cofactor mixed with soluble components, e.g., sugars (e.g., sucrose, mannitol, etc.), polymers (e.g., polyethylene glycol, hydroxypropyl cellulose, hydroxypropyl methyl cellulose, etc.), surfactants (sodium lauryl sulphate, chremophor, tweens, spans, pluronics, and the like), insoluble glidant components (microcrystalline cellulose, calcium phosphate, talc, fumed silica, and the like), coating material (examples of suitable coating materials are polyethylene glycol, hydroxypropyl methyl cellulose, wax, fatty acids, etc.), dispersions in suitable material (examples are wax, polymers, physiologically acceptable oils, soluble agents, etc.) or combinations of the above.

In some embodiments, the formulation is prepared such that a solid composition containing a substantially homogeneous mixture of the one or more cofactors is achieved, such that the one or more cofactors are dispersed evenly throughout the composition so that the composition may be readily subdivided into equally effective unit dosage forms such as tablets, pills and capsules.

The liquid forms, in which the formulations disclosed herein may be incorporated for administration orally or by injection, include aqueous solution, suitably flavored syrups, aqueous or oil suspensions, and flavored emulsions with edible oils such as cottonseed oil, sesame oil, coconut oil, or peanut oil as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin.

Liquid preparations for oral administration may take the form of, for example, solutions, syrups or suspensions, or they may be presented as a dry product for reconstitution with water or other suitable vehicles before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

For buccal administration, the formulation may take the form of tablets or lozenges formulated in conventional manners.

The one or more cofactors selected by the genetic makeup of the individual may be formulated for parenteral administration by injection, which includes using conventional catheterization techniques or infusion. Formulations for injection may be presented in unit dosage form, e.g., in ampules, or in multi-dose containers, with an added preservative. The compositions may take such forms as suspensions, solutions or emulsions in oily or aqueous vehicles, and may contain formulating agents such as suspending, stabilizing, and/or dispersing agents. Alternatively, the formulation comprising the one or more cofactors may be in powder form for reconstitution with a suitable vehicle, e.g., sterile pyrogen-free water, before use.

The formulations described herein can also be a slow release, sustained release, or controlled release formulation. For example, the formulation may release the one or more cofactors at a lower frequency or rate than it would be with an immediate release formulation (i.e., once a day versus twice a day or three times a day), which can improve the individual's compliance and caregiver convenience. These formulations can be particularly useful as they provide the one or more cofactors at a biologically effective amount from the onset of administration further improving compliance and adherence and enable the achievement of an effective steady-state concentration of the cofactor in a shorter period of time. Furthermore, the controlled release formulation allows for higher doses of a cofactor to be safely administered, again increasing the utility of these formulations for a variety of indications.

Using the controlled release dosage forms provided herein, the one or more cofactors can be released in its dosage form at a slower rate than observed for an immediate release formulation of the same quantity of cofactors. In some embodiments, the rate of change in the biological sample measured as the change in concentration over a defined time period from administration to maximum concentration for an controlled release formulation is less than about 80%, 70%, 60%, 50%, 40%, 30%, 20%, or 10% of the rate of the immediate release formulation. Furthermore, in some embodiments, the rate of change in concentration over time is less than about 80%, 70%, 60%, 50%, 40%, 30%, 20%, or 10% of the rate for the immediate release formulation.

In some embodiments, the rate of change of concentration over time is reduced by increasing the time to maximum concentration in a relatively proportional manner. For example, a two-fold increase in the time to maximum concentration may reduce the rate of change in concentration by approximately a factor of 2. As a result, the one or more cofactors may be provided so that it reaches its maximum concentration at a rate that is significantly reduced over an immediate release dosage form. The compositions of the present invention may be formulated to provide a shift in maximum concentration by 24 hours, 16 hours, 8 hours, 4 hours, 2 hours, or at least 1 hour. The associated reduction in rate of change in concentration may be by a factor of about 0.05, 0.10, 0.25, 0.5 or at least 0.8. In certain embodiments, this is accomplished by releasing less than about 30%, 50%, 75%, 90%, or 95% of the one or more cofactors into the circulation within one hour of such administration.

Optionally, the controlled release formulations exhibit plasma concentration curves having initial (e.g., from 2 hours after administration to 4 hours after administration) slopes less than 75%, 50%, 40%, 30%, 20% or 10% of those for an immediate release formulation of the same dosage of the same cofactor.

In some embodiments, the rate of release of the cofactor as measured in dissolution studies is less than about 80%, 70%, 60% 50%, 40%, 30%, 20%, or 10% of the rate for an immediate release formulation of the same cofactor over the first 1, 2, 4, 6, 8, 10, or 12 hours.

The controlled release formulations provided herein can adopt a variety of formats. In some embodiments, the formulation is in an oral dosage form, including liquid dosage forms (e.g., a suspension or slurry), and oral solid dosage forms (e.g., a tablet or bulk powder), such as, but not limited to those, those described herein.

The controlled release tablet of a formulation disclosed herein can be of a matrix, reservoir or osmotic system. Although any of the three systems is suitable, the latter two systems can have more optimal capacity for encapsulating a relatively large mass, such as for the inclusion of a large amount of a single cofactor, or for inclusion of a plurality of cofactors, depending on the genetic makeup of the individual In some embodiments, the slow-release tablet is based on a reservoir system, wherein the core containing the one or more cofactors is encapsulated by a porous membrane coating which, upon hydration, permits the one or more cofactors to diffuse through. Because the combined mass of the effective ingredients is generally in gram quantity, an efficient delivery system can provide optimal results.

Thus, tablets or pills can also be coated or otherwise compounded to provide a dosage form affording the advantage of prolonged action. For example, the tablet or pill can comprise an inner dosage an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings such materials including a number of polymeric acids and mixtures of polymeric acids with such materials as shellac, cetyl alcohol and cellulose acetate. In some embodiments, a formulation comprising a plurality of cofactors may have different cofactors released at different rates or at different times. For example, there can be additional layers of cofactors interspersed with enteric layers.

Methods of making sustained release tablets are known in the art, e.g., see U.S. Patent Publications 2006/051416 and 2007/0065512, or other references disclosed herein. Methods such as described in U.S. Pat. Nos. 4,606,909, 4,769,027, 4,897,268, and 5,395,626 can be used to prepare sustained release formulations of the one or more cofactors determined by the genetic makeup of an individual. In some embodiments, the formulation is prepared using OROS® technology, such as described in U.S. Pat. Nos. 6,919,373, 6,923,800, 6,929,803, and 6,939,556. Other methods, such as described in U.S. Pat. Nos. 6,797,283, 6,764,697, and 6,635,268, can also be used to prepare the formulations disclosed herein.

Furthermore, the methods of making the formulations disclosed herein can also be formulated to have a suitable and desirable taste, texture, or viscosity. For example, the formulation can comprise agents such as flavoring agents, coloring agents, and others can also be used. For example, pectic acid and the salt thereof, alginic acid and the salt thereof, organic acid, protective colloidal adhesive, pH controlling agent, stabilizer, a preservative, glycerin, alcohol,

In some embodiments, a formulation comprising one or more cofactors as determined by the genetic makeup of an individual can be formulated to have a suitable and desirable taste, texture, and viscosity for consumption, such as a food, food additive or beverage. Any suitable food carrier can be used in the present food compositions. Food carriers of the present invention include practically any food product. Examples of such food carriers include, but are not limited to food bars (granola bars, protein bars, candy bars, etc.), cereal products (oatmeal, breakfast cereals, granola, etc.), bakery products (bread, donuts, crackers, bagels, pastries, cakes, etc.), beverages (milk-based beverage, sports drinks, fruit juices, alcoholic beverages, bottled waters), pastas, grains (rice, corn, oats, rye, wheat, flour, etc.), egg products, snacks (candy, chips, gum, chocolate, etc.), meats, fruits, and vegetables.

In one embodiment, food carriers employed herein can mask the undesirable taste (e.g., bitterness), if present in one or more of the cofactors. Where desired, the food composition presented herein exhibit more desirable textures and aromas than that of the one or more cofactors.

In other embodiments, solid food carriers may be used according to the invention to obtain the present food compositions in the form of meal replacements, such as supplemented snack bars, pasta, breads, and the like. In yet other embodiments, semi-solid food carriers may be used according to the invention to obtain the present food compositions in the form of gums, chewy candies or snacks, and the like

In some embodiments, liquid food carriers, such as in the form of beverages, such as supplemented juices, coffees, teas, sodas, flavored waters, and the like can be used. For example, the beverage can comprise the formulation as well as a liquid component, such as various deodorant or natural carbohydrates present in conventional beverages. Examples of natural carbohydrates include, but are note limited to, monosaccharides such as, glucose and fructose; disaccharides such as maltose and sucrose; conventional sugars, such as dextrin and cyclodextrin; and sugar alcohols, such as xylitol and erythritol. Natural deodorant such as taumatin, stevia extract, levaudioside A, glycyrrhizin, and synthetic deodorant such as saccharin, aspartam et al., may also be used. Agents such as flavoring agents, coloring agents, and others can also be used. For example, pectic acid and the salt thereof, alginic acid and the salt thereof, organic acid, protective colloidal adhesive, pH controlling agent, stabilizer, a preservative, glycerin, alcohol, or carbonizing agents can also be used. Fruit and vegetables can also be used in preparing foods or beverages comprising the formulations discussed herein.

The formulations disclosed herein can also be provided to an individual, or health care of an individual along with a report on the genetic makeup of the individual, instructions on the dosage amount and administration of the formulations, lifestyle plan for the individual (such as recommended exercise or dietary habits), or information on the genetic variants and their correlation with cofactor-dependent enzyme deficiencies and cofactor remediable conditions.

### Business Methods

Also disclosed herein are business methods for determining an individual's genetic makeup by detecting the presence or absence of one or more genetic variants, determining a cofactor dependent enzyme deficiency, and providing a service of reporting the genetic makeup, cofactor dependent enzyme deficiency, or both to the individual or his/her agent. As used herein, “his/her agent” can be a guardian, healthcare manager, caretaker (e.g., doctor, nurse, medical assistant and the like), pharmacist, parent, attorney, doctor, accountant of the individual.

Also provided herein is a business method of determining an individual's genetic makeup by detecting the presence or absence of one or more genetic variants, selecting one or more cofactors for a formulation for the individual, and providing a service of reporting the genetic makeup, the formulation of the one or more cofactors, or both to the individual or a healthcare manager of the individual. The business methods can also include determining an individual's genetic makeup by detecting the presence or absence of one or more genetic variants, determining the amount of one or more cofactors for a formulation for the individual, and providing a service of reporting the genetic makeup, the formulation with the amount of the one or more cofactors, or both, to the individual or a healthcare manager of the individual.

In some embodiments, the methods further comprise incorporating one or more personal characteristics, such as those described herein, into determining the cofactor dependent enzyme deficiency, cofactor remediable condition, the one or more cofactors selected for a formulation, or the amount of one or more cofactors for a formulation, in the selection or determination step.

The information, such as the presence or absence of one or more genetic variants; the risk, predisposition, diagnosis, or prognosis of a metabolic condition, such as a cofactor dependent enzyme deficiency or cofactor remediable condition; the cofactor(s) selected for a formulation based on the genetic makeup of the individual; the amount of the cofactor(s) for a formulation; the dosing regimen, can be provided as a service or business to the individual or a health care manager of the individual. The health care manager may be the caretaker, physician, nurse, genetic counselor, or another healthcare professional. In some embodiments, the health care manager is a healthcare related company, such as a pharmaceutical company or nutraceutical company. The health care manager may administer the formulation to an individual, monitor the individual, or both.

For example, an individual may have a formulation comprising a plurality of cofactors selected based on their genetic makeup administered. A health care manager may observe the individual and determine whether the amounts of the cofactors in the formulation are suitable for the individual or should be altered. The resulting data and information can be used to correlate the amount of the one or more cofactors to the one or more genetic variants in the individual and stored in a database or computer readable medium, for future use in correlating the amounts of the cofactors to the genetic variants in other individuals. The information can be used or sold to a nutraceutical company.

In another embodiment, a health care manager may be a pharmaceutical company. The health care manager may be interested in determining whether a formulation of one or more cofactors and the amount of the one or more cofactors, as selected by the genetic makeup for the individual may enhance the efficacy of a therapeutic. As such, the pharmaceutical company may run clinical trials monitoring the different formulations as well as therapeutics being administered to an individual and their effects.

In some embodiments, the methods disclosed herein further comprise providing the formulation of the one or more cofactors as selected by the genetic makeup of the individual, to the individual or a health care manager of the individual. The formulation can comprise amounts of the one or more cofactors as determined by the genetic makeup of the individual. Furthermore, the selection of the one or more cofactors, the amount of the one or more cofactors, or both, can also be based on one or more personal characteristics of the individual as discussed above. The formulation may be produced by the methods disclosed herein. Furthermore, the formulation can be manufactured by the same or different party performing the analysis of the genetic makeup of the individual.

One or more parties, such as the same or different party or parties, can collect or obtain the biological sample from an individual, detect the one or more genetic variants from the biological sample, determine the cofactor dependent enzyme deficiency based on the one or more genetic variants, determine the one or more cofactors for a formulation for the individual based on the genetic makeup of the individual, determine the amount of one or more cofactors for a formulation for the individual based on the genetic makeup of the individual, reporting the results of any of the aforementioned steps (such as the presence or absence of genetic variants, the risk or predisposition to a cofactor-dependent enzyme deficiency or cofactor remediable condition, the formulation), manufacture the formulation, or provide the formulation.

The one or more parties may charge a fee for each of the processes or services they provide, or for a subset of the services or processes they provide. There may be different levels of fees or charges based on the level of service. For example, a party detecting the one or more genetic variants may provide a service of detecting more genetic variants for a higher fee.

Also provided herein is a method of classifying an individual with a cofactor remediable condition. For example, based on the genetic makeup of the individual, the individual may be classified within a scale ranging from low to high risk of a cofactor-dependent enzyme deficiency or a cofactor remediable condition. The classification system may be a numerical scoring system, such as ranging from 1 through 5, where 1 represents a low risk and 5 represents a high risk. In other embodiments, the classification system is a descriptive or alphabetical system. For example, the system may classify an individual as “Low Risk,” “Medium Risk,” or “High Risk” for a cofactor-dependent enzyme deficiency. Alternatively, the system may classify individuals based on an alphabetical system, such as from A through E, where an “A” rating represent an individual has a low risk for a cofactor-dependent enzyme deficiency and an “E” rating represents an individual with a high risk for a cofactor-dependent enzyme deficiency.

The classification system can also be represented with different colors, symbols, or other visuals. For example, a color system of green, orange and red may be used, where green is used to represent low risk, orange to represent medium risk, and red to represent high risk, of a cofactor dependent enzyme deficiency. The various means of classifying can be combined. For example, the classification system can combine a color scheme with a descriptive scheme. The classification can be provided in a report that is presented to the individual or a healthcare manager of the individual. The classification can be provided by the same or different party reporting other results from the individual's genetic makeup.

The methods disclosed herein can comprise providing one or more reports to an individual or a healthcare manager of the individual. For example, the one or more reports can include, but not be limited to, information such as the genetic makeup of the individual, such as the presence or absence of one or more genetic variants; the personal characteristics of the individual that was incorporated into determining the one or more cofactors or the amount of one or more cofactors for a formulation for the individual; the risk, predisposition, diagnosis, or prognosis of a metabolic condition, such as a cofactor dependent enzyme deficiency or cofactor remediable condition based on the genetic makeup of the individual; the one or more cofactors selected for a formulation for the individual; or the amount of the one or more cofactor determined for a formulation for the individual. The methods disclosed herein can also provide a personalized nutritional or dietary plan in one or more reports to the individual.

The reports may be provided in a digital format, such as accessible by a website. The reports may also be provided in a digital format stored on a computer readable medium. The report can also be provided in paper form. The reports may be transmitted over a network to an individual or healthcare manager of the individual. In some embodiments, updated reports are generated and provided to an individual or healthcare manager of the individual. For example, new genetic variants and correlations may be obtained through scientific research, published literature or other sources. The new genetic variants and correlations may be genetic variants that were not previously known to be associated with a cofactor-dependent enzyme deficiency. Alternatively, the genetic variants may have been known to be associated with a cofactor-dependent enzyme deficiency, but the correlation may be weaker or stronger than previously discovered. In another embodiment, the genetic variant may have been known and previously associated with a cofactor-dependent enzyme deficiency but new results indicate a new association with a different cofactor-dependent enzyme deficiency.

The new genetic variants and correlations can be used to generate new results, such as a different cofactor or combination of cofactors in the formulation for an individual as compared to an original formulation generated for the individual. In another embodiment, different amounts of one or more cofactors in a formulation for an individual results from the new genetic variants.

In another embodiment, updated reports are generated based on updated personal characteristics of an individual. For example, an initial report was generated for a pregnant female. After giving birth, the individual can have an updated report showing that the recommended formulation of one or more cofactors has changed given the female is no longer pregnant. In another embodiment, an individual can have an updated report based on discovery a new medical condition change in dietary plan, as compared to when an initial report was first generated. The updated report can contain updated formulations, such as different cofactors or different amounts of the cofactors.

### Computer Systems

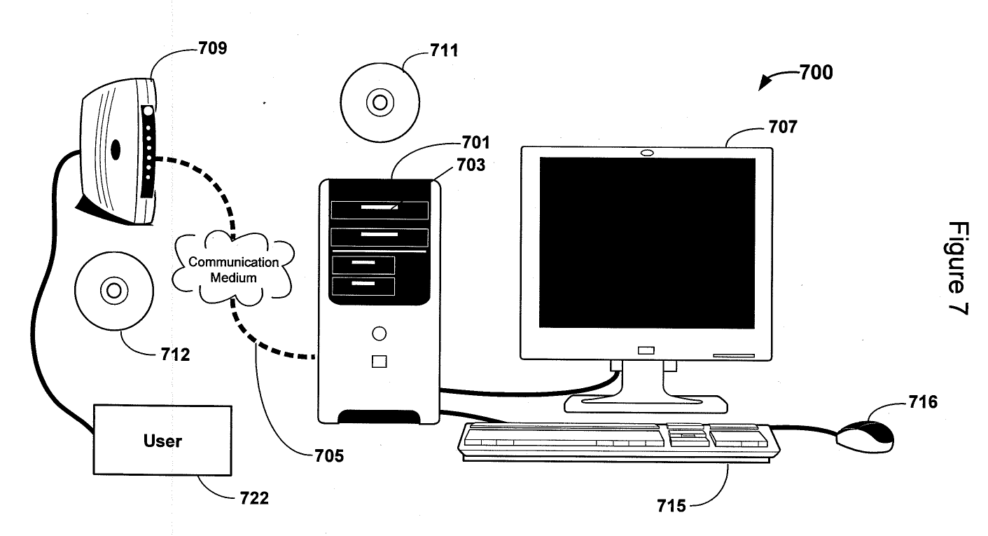

In yet another aspect of the present invention, computer systems for performing one or more of the methods disclosed herein is provided. Accordingly, the methods disclosed herein can be performed by a representative logic device such as a computer system (or digital device). An example of a computer system is depicted in FIG. 7, which can receive and store data generated from the analysis of an individual's biological sample. For example, the computer system can store data such as the absence or presence of the one or more genetic variants in a biological sample, such as those listed in Tables A-X. Furthermore, the representative device or computer system can also analyze the data to determine a formulation of one or more cofactors for an individual, determine the amount of one or more cofactors in formulation for the individual, determine the risk or predisposition of a cofactor-dependent enzyme deficiency, determine the risk or predisposition of a cofactor-remediable condition, classify the individual in different risk categories for a cofactor-dependent enzyme deficiency or cofactor-remediable condition, determine a personalized lifestyle recommendation plan for the individual, generate instructions for taking the formulation determined by the computer, or generate a report based any of the above determinations or analyses.

In some embodiments, one or more computer systems may be used to perform one or more of the aforementioned processes. For example, a network of computer systems may be used, wherein the network of computer systems can be in the same location or different location. The computer systems may be linked such that the results, data, or information from one computer system can be transmitted, received, and/or outputted to one or more other computer systems. The transmission can be a network connection, a wireless connection or an internet connection. Such a connection can provide for communication over the World Wide Web. Data relating to the present invention can be transmitted over such networks.

FIG. 7 shows a representative computer system (or digital device), where the computer system 700 may be understood as a logical apparatus that can read instructions from media 711 and/or network port 705, which can optionally be connected to server 709 having fixed media 712. The system shown in FIG. 7 includes CPU 701, disk drives 703, optional input devices such as keyboard 715 and/or mouse 716 and optional monitor 707. Data communication can be achieved through the indicated communication medium to a server 709 at a local or a remote location.

The communication medium can include any means of transmitting and/or receiving data. For example, the communication medium can be a network connection, a wireless connection or an internet connection. Such a connection can provide for communication over the World Wide Web. It is envisioned that data relating to the present invention can be transmitted over such networks or connections for reception and/or review by a party 722. The receiving party 722 can be but is not limited to an individual or a health care manager. In one embodiment, a computer-readable medium includes a medium suitable for transmission of a result. The medium can include a result regarding a formulation, risk or predisposition for a cofactor remediable condition, or a personalized lifestyle recommendation plan for of an individual, derived using the methods described herein.

The computer system can analyze the genetic data obtained from a biological sample of an individual by correlating the presence or absence of genetic variants with the risk, predisposition, or diagnosis of a cofactor-dependent enzyme deficiency or cofactor remediable condition. For example, the computer system can have code for correlating at least one genetic variant of a gene in a metabolic pathway to a cofactor-dependent enzyme deficiency, or a cofactor-remediable metabolic condition. The computer system can have a database of genetic variants and their association or correlation to a cofactor-dependent enzyme deficiency, or a cofactor-remediable metabolic condition, such as the odds ratio or relative risk of having the deficiency or condition if an individual has a particular genetic variant. The computer system can then determine an individual's risk or predisposition, or prognosis of a cofactor-dependent enzyme deficiency, or a cofactor-remediable metabolic condition by comparing the data received from a biological sample and comparing it to the database of genetic variants and correlations.

The computer system can also be used to select one or more cofactors for a formulation based on the genetic makeup of an individual, such as based on the data generated from the analysis of an individual's biological sample. For example, the computer system can comprise code for determining a cofactor formulation for an individual with or without a particular genetic variant. The computer system can also be used to determine the amount of one or more cofactors for a formulation based on the genetic makeup of an individual. For example, the computer system can comprise code for determining the amount of one or more cofactors in a formulation for an individual based on the presence or absence of a particular genetic variant.

In some embodiments, the computer system is able to analyze and correlate a plurality for genetic variants. For example, the computer system can comprise code for correlating a plurality of genetic variants to determine an individual's risk or predisposition, or prognosis of a cofactor-dependent enzyme deficiency, or a cofactor-remediable metabolic condition. The computer system can also comprise code for correlating a plurality of genetic variants the amount of one or more cofactors in a formulation for the individual. Furthermore, the computer system can further incorporate personal characteristics into determining the risk or predisposition of a cofactor-dependent enzyme deficiency, or a cofactor-remediable metabolic condition; one or more cofactors that should be in a formulation for the individual; or the amount of one or more cofactors for a formulation for an individual.

The computer system can also comprise code for generating reports, outputting reports, and transmitting the reports. The transmission of reports can be over a network, such as a secure network. Similarly, data obtained from analysis of an individual's biological sample can be received and sent by transmitting the data over a network, such as a secure network. In some embodiments, the report is delivered to an individual or health care manager of the individual via the Internet. The report can be transmitted with the use of a unique identifier code. The report can be transported to a computer, such as a home computer, work computer, or personal digital assistant or personal digital device, such as a SmartPhone, such as a Blackberry®, iPhone®, or any other device available.

Furthermore the code described herein can be encoded on a computer readable medium, which can form part of the computer system. For example, a computer system disclosed herein can comprise a first dataset on a data processing device, wherein the first dataset comprises information correlating the presence of a genetic variant of the individual to a risk of a cofactor-dependent enzyme deficiency or cofactor remediable condition. The computer system further comprises a second dataset on a data processing device, wherein the second dataset comprises information matching the cofactor-dependent enzyme deficiency or cofactor remediable condition with a formulation of one or more cofactors. In some embodiments, the computer system comprises a dataset with information that correlates a plurality of genetic variants to a risk of a cofactor-dependent enzyme deficiency or cofactor remediable condition. Furthermore, the computer system can further incorporate personal characteristics into matching the cofactor-dependent enzyme deficiency or cofactor remediable condition with a formulation of one or more cofactors. The information on the one or more personal characteristics can form another dataset on a data processing device of the computer system described herein. The computer system can also comprise an additional dataset on lifestyle recommendations that are correlated to one or more genetic variants, one or more cofactor-dependent enzyme deficiencies or cofactor remediable conditions, one or more formulations of one or more cofactors, or one or more personal characteristics of an individual.

In another embodiment, the first dataset comprises information relating to one or more genetic variants of one or more genes correlated with a cofactor-dependent enzyme deficiency or cofactor remediable condition. For example, the gene may be involved in, but not be limited to, the thiamine metabolic pathway, riboflavin metabolic pathway, vitamin B6 metabolic pathway, nicotinate and nicotinamide metabolic pathway, pantothenate and CoA biosynthesis pathway, biotin metabolic pathway, lipoic metabolic pathway, folate/homocysteine metabolic pathway, retinol metabolic pathway, porphyrin metabolic pathway, ubiquinone and other terpenoid-quinone biosynthesis pathway. The genetic variant can be of a gene in the pathway for metabolizing Vitamin A (retinol), Vitamin C (ascorbic acid), Vitamin D (calciferol), Vitamin E, Vitamin K (phylloquinone), Vitamin B1 (Thiamin), Vitamin B2 (riboflavin), Vitamin B3 (niacin), Vitamin B6 (pyridoxine), Vitamin B9 (folate/folic acid), Vitamin B12 (tocopherol), Vitamin B7 (biotin), Vitamin B5 (panthothenic acid), or choline.

For example, the one or more genes may be a gene selected from the folate pathway, such as AHCY, AHCYL1, AHCYL2, ALDH1L1, ALDHL2, AMT, ATIC, BHMT1, BHMT2, CBS, CTH, DHFR, DMGDH, FPGS, FTCD, GART, GGH, MAT1A, MAT2A, MTFMT, MTHFD1, MTHFD2, MTHFR, MTHFS, MTR, MTRR, NAALAD2, SARDH, SHMT1, SHMT2, or TYMS. The first data set can comprise a plurality of genetic variants, such as at least 2, 3, 4, 5, 6, 7, 8, 9, 10, 15, 20, 25, 30, 40, 50, 75, or 100 genetic variants. The genetic variants can be of the same gene, of different genes in the same metabolic pathway, or of different genes in different metabolic pathway. The genetic variant, such as a SNP, can be selected a genetic variant of MTHFR, ATIC, MTHFS, MAT1A, MAT2A, GART, AHCY, AMT, CBS, CTH, DHFR, FPGS, MTHFD1, MTHFD2, MTR, SHMT1, SHMT2, or TYMS. For example, the genetic variant can be selected from Tables A-X.

The computer system can use a first dataset comprising the information on genetic variants to assign a risk, predisposition or susceptibility of a cofactor-dependent enzyme deficiency or cofactor remediable condition for the one or more genetic variants identified in a sample, and then use the second dataset to assign a formulation comprising a cofactor, plurality of cofactors, or amount of one or more cofactors. In some embodiments, the computer system can use another dataset, such as a third dataset.

For example, the computer system can comprise a third dataset that comprises information on personal characteristics. The computer system can use the third dataset of personal characteristics to modify the risk, predisposition or susceptibility of a cofactor-dependent enzyme deficiency or cofactor remediable condition obtained from using the first dataset, and/or assign a formulation comprising a cofactor, plurality of cofactors, or amount of one or more cofactors, by modifying the results obtained after using the second dataset.

For example, an individual has genetic variants indicating a high risk of a cofactor remediable condition. The individual also has a high intake of the cofactor. Using the first dataset, the computer system would generate a result in determining that the individual has a high risk of the cofactor remediable condition, but using the third dataset with personal characteristics, the risk is modified because the individual's diet has a high intake of that cofactor. In another embodiment, the results of the second dataset would indicate a formulation of a high amount of a cofactor, but taking into account the third dataset where the individual has a high intake of the cofactor, the formulation would be modified to lower the amount of the cofactor.

In yet another embodiment, the third dataset can comprise lifestyle recommendations, which can provide personalized lifestyle recommendations for an individual. The personalized lifestyle recommendation can be, but not limited to, a nutrition plan, dietary plan, or exercise plan. For example, the personalized lifestyle recommendation plan can include, but not be limited to, recommended minimum and/or maximum amounts of various cofactors, such as specific vitamins or minerals, what foods or drinks should be included in the individual's diet, what types of foods should be avoided, and what types of exercise should be included. The computer system can use the third dataset of lifestyle recommendations to match lifestyle recommendations to a cofactor-dependent enzyme deficiency or cofactor remediable condition as determined by the computer system using the first dataset. The third dataset of lifestyle recommendations can also be used to provide personalized recommendations based on the formulation comprising a cofactor, plurality of cofactors, or amount of one or more cofactors, obtained after using the second dataset.

For example, an individual has genetic variants indicating a high risk of a cofactor remediable condition. Using the first dataset, the computer system would generate a result in determining that the individual has a high risk of the cofactor remediable condition, and using the third dataset with lifestyle recommendations, one or more lifestyle recommendations would be matched with the individual's risk for the cofactor remediable condition, such as providing a dietary plan for the individual including foods high in the cofactor. In another embodiment, the results of the second dataset would indicate a formulation of a high amount of the cofactor, and based on the formulation being taken by the individual, one or more lifestyle recommendations would be generated, such as a dietary plan that complements the individual's intake of the formulation.

In yet another embodiment, the computer system described herein uses at least 4 datasets, such as a first a first dataset comprising the information on genetic variants to assign a risk, predisposition or susceptibility of a cofactor-dependent enzyme deficiency or cofactor remediable condition for the one or more genetic variants identified in a sample; a second dataset to assign a formulation comprising a cofactor, plurality of cofactors, or amount of one or more cofactors; a third dataset of personal characteristics to modify the risk, predisposition or susceptibility of a cofactor-dependent enzyme deficiency or cofactor remediable condition obtained from using the first dataset, or a formulation comprising one or more cofactors; and a fourth dataset to provide personalized lifestyle recommendations.

In one embodiment, the computer system using at least 4 datasets uses the first dataset comprising the information on genetic variants to assign a risk, predisposition or susceptibility of a cofactor-dependent enzyme deficiency or cofactor remediable condition for the one or more genetic variants identified in a sample. The computer system then uses the second dataset to assign a formulation comprising a cofactor, plurality of cofactors, or amount of one or more cofactors. The third dataset of personal characteristics is then used to modify the risk, predisposition or susceptibility of a cofactor-dependent enzyme deficiency or cofactor remediable condition obtained from using the first dataset and/or a formulation comprising one or more cofactors obtained from using the second dataset. The fourth dataset of lifestyle recommendations is then used to generate at least lifestyle recommendation by matching one or more lifestyle recommendations to the modified risk or susceptibility of a cofactor-dependent enzyme deficiency or cofactor remediable condition obtained from using the third dataset; and/or a formulation comprising one or more cofactors obtained from using the third dataset.

For example, an individual has genetic variants indicating a high risk of a cofactor remediable condition. The individual also has a high intake of the cofactor. Using the first dataset, the computer system would generate a result in determining that the individual has a high risk of the cofactor remediable condition, but using the third dataset with personal characteristics, the risk is modified because the individual's diet has a high intake of that cofactor. The results of the second dataset would indicate a formulation of a high amount of a cofactor, but taking into account the third dataset of personal characteristics, where the individual has a high intake of the cofactor the formulation would be modified to lower the amount of the cofactor. The fourth dataset of lifestyle recommendations would then match one or more lifestyle recommendations to the modified risk of the cofactor remediable condition resulting from the use of the third dataset and/or match one or more lifestyle recommendations to the modified formulation of cofactors resulting from the use of the third dataset.

In yet other embodiments, the computer system further comprises a dataset comprising information to classify an individual, such as classification schemes as described herein. The classification dataset can be used in combination with any dataset described herein. For example, using the first dataset, the computer system can generate a result in determining that the individual has a high risk of the cofactor remediable condition. Using the dataset comprising information on classifying the individual, a classification category would be matched with the risk obtained from using the first dataset. In another embodiment, the risk obtained from using the first dataset is modified by using a dataset with personal characteristics. Using the dataset comprising information on classifying the individual, a classification category would be matched with the modified risk obtained using the dataset of personal characteristics. In yet another embodiment, after using the dataset to classify an individual, a dataset comprising information on the formulation of one or more cofactors, or the amount of one or more cofactors can be used to match the one or more cofactors and/or the amount of the one or more cofactors based on the classification of the individual. In another embodiment, a dataset comprising lifestyle recommendations can be used to match one or more lifestyle recommendations to an individual based on the classification of the individual.

Furthermore, any one of the datasets described herein can be updated. For example, the dataset of genetic variants and their correlations to a cofactor-dependent enzyme deficiency or cofactor remediable condition can be updated with new genetic variants and correlations may be obtained through scientific research, published literature or other sources. The new genetic variants and correlations may be novel genetic variants that were not previously known to be associated with a cofactor-dependent enzyme deficiency or cofactor remediable condition. Alternatively, the genetic variants may have been known to be associated with a cofactor-dependent enzyme deficiency or cofactor remediable condition, but the correlation may be weaker or stronger than previously discovered. In another embodiment, the genetic variant may have been known and previously associated with a cofactor-dependent enzyme deficiency or cofactor remediable condition but new results indicate a new association with a different cofactor-dependent enzyme deficiency or cofactor remediable condition. The new genetic variants and correlation can be updated in the first dataset described herein.

In yet another example, a dataset used to assign a formulation comprising a cofactor, plurality of cofactors, or amount of one or more cofactors can be updated with new cofactors, different cofactors, or different amounts of cofactors than was originally used to assign to a given risk or predisposition of a cofactor-dependent enzyme deficiency or cofactor remediable condition.

A dataset of personal characteristics can also be updated to include new or modified characteristics of an individual or to update with personal characteristics that were not previously known to be associated with a cofactor-dependent enzyme deficiency or cofactor remediable condition, or the correlation may be weaker or stronger than previously discovered. Datasets can comprise information on lifestyle recommendations and classification of an individual can also be updated to reflect new scientific research, published literature or other sources. For example, lifestyle recommendations can be modified or correlated to different cofactor remediable conditions.

The computer system described herein can also generate one or more reports comprising one or more of the following: the genetic makeup of the individual, such as the genetic variants present or absent from an individual's sample; the individual's risk of a cofactor-dependent enzyme deficiency or cofactor remediable condition; the formulation comprising one or more cofactors based on the individual's genetic makeup; the amount of the one or more cofactors for a formulation based on the individual's genetic makeup; the personal characteristics of an individual taken into account for determining the risk of a cofactor remediable condition or cofactor-dependent enzyme deficiency or the formulation for an individual; one or more lifestyle recommendation or personalized lifestyle recommendation plan; and the classification of the individual.

The reports may be provided in a digital format, such as accessible by a website. The reports may also be provided in a digital format stored on a computer readable medium. The report can also be provided in paper form. The reports can be sent by computer, such as by transmission over a network, to one or more parties, such as the individual, a health care manager of the individual, or another third party, such as a manufacturer that can produce the formulation. The formulation can then be shipped or sold to the individual or a caretaker of the individual. In some embodiments, updated reports are generated and provided to an individual or healthcare manager of the individual. The report can be transmitted with the use of a unique identifier code. The report can be transported to a computer, such as a home computer, work computer, or personal digital assistant or personal digital device, such as a SmartPhone, such as a Blackberry®, iPhone®, or any other device available.

TABLE A
MTHFR Variants

| GENE_position | Exon | Type | Function | Location | dB SNP id | Change |
|---|---|---|---|---|---|---|
| MTHFR_3921 | 2 | SNP | non-coding | 5′-UTR | rs34889587 | C/T |
| MTHFR_4059 | 2 | SNP | Synonymous | P39P | rs2066470 | C/T |
| MTHFR_4078 | 2 | SNP | Nonsynonymous | R46W | | C/T |
| MTHFR_4145 | 2 | SNP | Nonsynonymous | R68Q | rs2066472 | A/G |
| MTHFR_4181 | 2 | SNP | non-coding | IVS2 + 3 | rs1413355 | A/G |
| MTHFR_4234 | 2 | SNP | non-coding | IVS + 56 | | A/G |
| MTHFR_5699 | 3 | SNP | Synonymous | D92D | rs45546035 | C/T |
| MTHFR_5733 | 3 | SNP | Nonsynonymous | D104Y | | G/T |
| MTHFR_5840 | 3 | SNP | Synonymous | T139T | rs2066466 | A/G |
| MTHFR_5872 | 3 | SNP | Nonsynonymous | L150P | | C/T |
| MTHFR_6642 | 4 | SNP | non-coding | IVS3 − 95 | | C/T |
| MTHFR_6651 | 4 | SNP | non-coding | IVS3 − 86 | rs13306567 | C/G |
| MTHFR_6657 | 4 | SNP | non-coding | IVS3 − 80 | | C/T |
| MTHFR_6658 | 4 | SNP | non-coding | IVS3 − 79 | rs2066471 | A/G |
| MTHFR_6661 | 4 | SNP | non-coding | IVS3 − 76 | rs2066469 | A/G |
| MTHFR_6681 | 4 | indel | non-coding | IVS3 − 56 | | −/+ deletion AG |
| MTHFR_6774 | 4 | SNP | Synonymous | G171G | | A/C |
| MTHFR_10738 | 5 | SNP | Nonsynonymous | A222V | rs59514310 | C/T |
| MTHFR_10906 | 5 | SNP | non-coding | IVS5 + 53 | | C/T |
| MTHFR_11656 | 6 | SNP | non-coding | IVS5 − 55 | | C/T |
| MTHFR_11668 | 6 | SNP | non-coding | IVS5 − 43 | | C/T |
| MTHFR_11836 | 6 | SNP | Synonymous | A302A | rs13306555 | C/T |
| MTHFR_11902 | 6 | SNP | Synonymous | N324N | | C/T |
| MTHFR_12044 | 6 | SNP | non-coding | IVS6 + 83 | rs2066467 | A/G |
| MTHFR_12190 | 7 | SNP | non-coding | IVS6 − 6 | rs2066464 | A/G |
| MTHFR_12220 | 7 | SNP | Synonymous | S352S | rs2066462 | C/T |
| MTHFR_12232 | 7 | SNP | Synonymous | K356K | | A/G |
| MTHFR_12361 | 7 | SNP | non-coding | IVS7 + 31 | rs1994798 | C/T |
| MTHFR_12445 | 8 | SNP | non-coding | IVS7 − 76 | rs12121543 | G/T″ |
| MTHFR_12618 | 8 | SNP | Nonsynonymous | G422R | rs45571736 | A/G |
| MTHFR_12622 | 8 | indel | Frame Shift | E423fs | | −/+ insertion G |
| MTHFR_12641 | 8 | SNP | Nonsynonymous | E429A | rs1801131 | A/C |
| MTHFR_12660 | 8 | SNP | Synonymous | F435F | rs57431061 | C/T |
| MTHFR_12759 | 8 | SNP | non-coding | IVS8 + 57 | | A/G |
| MTHFR_13040 | 9 | SNP | Nonsynonymous | R473W | | C/T |
| MTHFR_13099 | 9 | SNP | Synonymous | P492P | rs35653697 | A/G |
| MTHFR_13192 | 9 | SNP | non-coding | IVS9 + 39 | rs45515693 | C/T |
| MTHFR_14593 | 10 | SNP | non-coding | IV9 − 88 | | G/T |
| MTHFR_14601 | 10 | SNP | non-coding | IVS9 − 80 | rs17375901 | A/G |
| MTHFR_14612 | 10 | SNP | non-coding | IVS9 − 69 | | A/G |
| MTHFR_14705 | 10 | SNP | Nonsynonymous | R519C | rs45496998 | C/T |
| MTHFR_14814 | 10 | SNP | non-coding | IVS10 + 32 | rs45497396 | C/T |
| MTHFR_14817 | 10 | SNP | non-coding | IVS10 + 35 | rs58018465 | A/G |
| MTHFR_16114 | 12 | SNP | non-coding | IVS11 − 48 | rs56932901 | C/G |
| MTHFR_16136 | 12 | SNP | non-coding | IVS11 − 26 | rs45622739 | A/G |
| MTHFR_16170 | 12 | SNP | Synonymous | A587A | | C/T |
| MTHFR_16190 | 12 | SNP | Nonsynonymous | R594Q | rs58316272 | A/G |
| MTHFR_16367 | 12 | SNP | Nonsynonymous | T653M | rs35737219 | C/T |
| MTHFR_16368 | 12 | SNP | Synonymous | T653T | rs45572531 | A/G |
| MTHFR_16401 | 12 | SNP | non-coding | 3′UTR | | C/T |
| MTHFR_16451 | 12 | SNP | non-coding | 3′UTR | | C/T |

TABLE B
ATIC Variants

| GENE_position | Exon | Type | Function | Location | dB SNP id | Change |
|---|---|---|---|---|---|---|
| ATIC_1089 | 1 | SNP | non-coding | 5′UTR | rs28366034 | C/T |
| ATIC_1100 | 1 | SNP | non-coding | 5′UTR | | C/T |
| ATIC_1114 | 1 | SNP | non-coding | 5′UTR | | C/T |
| ATIC_1116 | 1 | SNP | non-coding | 5′UTR | rs4535042 | T/C |
| ATIC_1133 | 1 | SNP | non-coding | 5′UTR | rs28366035 | C/G/T |
| | | | | | | (TRIALLELE) |
| ATIC_1152 | 1 | SNP | non-coding | 5′UTR | rs11550205 | C/T |
| ATIC_1160 | 1 | SNP | non-coding | 5′UTR | rs11550203 | C/T |
| ATIC_1179 | 1 | SNP | Nonsynonymous | A2V | | C/T |
| ATIC_1244 | 1 | indel | non-coding | IVS1 + 50 | | −/+ |
| | | | | | | insertion C |
| ATIC_1270 | 1 | SNP | non-coding | IVS1 + 76 | | C/T |
| ATIC_1288 | 1 | SNP | non-coding | IVS1 + 94 | | G/A |
| ATIC_1301 | 1 | SNP | non-coding | IVS1 + 107 | | G/A |
| ATIC_1380 | 2 | SNP | non-coding | IVS1 − 151 | | A/G |
| ATIC_1396 | 2 | SNP | non-coding | IVS1 − 135 | | G/C |
| ATIC_1453 | 2 | SNP | non-coding | IVS1 − 78 | | C/T |
| ATIC_1506 | 2 | SNP | non-coding | IVS1 − 25 | | T/C |
| ATIC_1689 | 2 | SNP | non-coding | IVS2 + 32 | | T/A |
| ATIC_7227 | 3 | SNP | Nonsynonymous | G62R | | G/C |
| ATIC_7232 | 3 | indel | Nonsynonymous | G63fs | | −/+ |
| | | | | | | insertion G |
| ATIC_7388 | 3 | SNP | non-coding | IVS3 + 121 | | T/A |
| ATIC_8756 | 4 | SNP | Nonsynonymous | N94S | | A/G |
| ATIC_8793 | 4 | SNP | non-coding | IVS4 + 28 | rs16853782 | A/G |
| ATIC_8808 | 4 | SNP | non-coding | IVS4 + 43 | | G/A |
| ATIC_14099 | 5 | SNP | non-coding | IVS4 − 176 | | C/T |
| ATIC_14136 | 5 | SNP | non-codinq | IVS4 − 139 | rs3772077 | A/G |
| ATIC_14140 | 5 | SNP | non-coding | IVS4 − 135 | | C/A |
| ATIC_14144 | 5 | SNP | non-coding | IVS4 − 131 | | C/T |
| ATIC_14156 | 5 | SNP | non-coding | IVS4 − 119 | rs3772078 | A/G |
| ATIC_14183 | 5 | SNP | non-coding | IVS4 − 92 | | C/T |
| ATIC_14229 | 5 | SNP | non-coding | IVS4 − 46 | | A/G |
| ATIC_14238 | 5 | SNP | non-coding | IVS4 − 37 | | C/T |
| ATIC_14245 | 5 | SNP | non-coding | IVS4 − 30 | | A/C |
| ATIC_14260 | 5 | SNP | non-coding | IVS4 − 15 | | G/T |
| ATIC_14331 | 5 | SNP | Nonsynonymous | T116S | rs2372536 | G/C |
| ATIC_14489 | 5 | SNP | non-coding | IVS5 + 126 | | G/A |
| ATIC_14965 | 6 | SNP | non-coding | IVS5 − 56 | rs7563206 | C/T |
| ATIC_14970 | 6 | SNP | non-coding | IVS5 − 51 | | C/T |
| ATIC_15003 | 6 | SNP | non-coding | IVS5 − 18 | | G/A |
| ATIC_15040 | 6 | SNP | Synonymous | R133R | | A/G |
| ATIC_15043 | 6 | SNP | Synonymous | A134A | | T/C |
| ATIC_15149 | 6 | SNP | Nonsynonymous | T170A | | A/G |
| ATIC_15240 | 6 | SNP | non-coding | IVS6 + 68 | | A/G |
| ATIC_15826 | 7 | SNP | non-coding | IVS6 − 30 | rs6751557 | C/T |
| ATIC_15844 | 7 | SNP | non-coding | IVS6 − 12 | | C/T |
| ATIC_16063 | 7 | SNP | non-coding | IVS7 + 51 | | G/A |
| ATIC_21363 | 8 | SNP | non-coding | IVS7 − 53 | | A/G |
| ATIC_21372 | 8 | SNP | non-coding | IVS7 − 44 | | T/G |
| ATIC_21400 | 8 | SNP | non-coding | IVS7 − 16 | | A/G |
| ATIC_21521 | 8 | indel | Nonsynonymous | F265fs | | −/+ deletion T |
| ATIC_21611 | 8 | SNP | non-coding | IVS8 + 70 | | T/A |
| ATIC_22187 | 9 | SNP | non-coding | IVS8 − 197 | | G/A |
| ATIC_22273 | 9 | SNP | non-coding | IVS8 − 111 | | A/G |
| ATIC_22282 | 9 | indel | non-coding | IVS8 − 103 | | −/+ |
| | | | | | | insertion A |
| ATIC_22283 | 9 | SNP | non-coding | IVS8 − 102 | rs12995526 | C/T |
| ATIC_22291 | 9 | SNP | non-coding | IVS8 − 94 | | G/A |
| ATIC_22342 | 9 | SNP | non-coding | IVS8 − 43 | | A/G |
| ATIC_22361 | 9 | SNP | non-coding | IVS8 − 24 | rs10179873 | A/G |
| ATIC_22512 | 9 | SNP | non-coding | IVS9 + 20 | | T/G |
| ATIC_22519 | 9 | SNP | non-coding | IVS9 + 27 | | G/T |
| ATIC_22538 | 9 | SNP | non-coding | IVS9 + 46 | | A/G |
| ATIC_22564 | 9 | indel | non-coding | IVS9 + 72 | | −/+ deletion |
| | | | | | | GGA |
| ATIC_22589 | 9 | SNP | non-coding | IVS9 + 97 | | G/T |
| ATIC_22686 | 9 | SNP | non-coding | IVS9 + 194 | rs10932606 | C/T |
| ATIC_22737 | 9 | SNP | non-coding | IVS9 + 245 | | A/G |
| ATIC_24992 | 11 | indel | non-coding | IVS10 − 79 | | −/+ |
| | | | | | | insertion G |
| ATIC_25009 | 11 | SNP | non-coding | IVS10 − 62 | | A/G |
| ATIC_25220 | 11 | SNP | non-coding | IVS11 + 60 | rs13002576 | G/C |
| ATIC_27609 | 12 | SNP | non-coding | IVS11 − 206 | rs16853823 | A/G |
| ATIC_27739 | 12 | SNP | non-coding | IVS11 − 76 | rs6721444 | C/A |
| ATIC_27757 | 12 | SNP | non-coding | IVS11 − 58 | | A/G |
| ATIC_27855 | 12 | SNP | Nonsynonymous | T380I | | C/T |
| ATIC_27985 | 12 | SNP | non-coding | IVS12 + 42 | | T/C |
| ATIC_28015 | 12 | SNP | non-coding | IVS12 + 72 | | A/G |
| ATIC_33785 | 13 | SNP | non-coding | IVS12 − 30 | rs13010249 | A/G |
| ATIC_33901 | 13 | SNP | Synonymous | N438N | | C/T |
| ATIC_33919 | 13 | SNP | non-coding | IVS13 + 12 | | G/A |
| ATIC_33920 | 13 | SNP | non-coding | IVS13 + 13 | | T/C |
| ATIC_33933 | 13 | SNP | non-coding | IVS13 + 26 | | C/T |
| ATIC_35723 | 14 | SNP | non-coding | IVS13 − 72 | | G/A |
| ATIC_35737 | 14 | SNP | non-coding | IVS13 − 58 | | C/A |
| ATIC_35742 | 14 | SNP | non-coding | IVS13 − 53 | | G/C |
| ATIC_35840 | 14 | SNP | Nonsynonymous | R456S | | C/A |
| ATIC_35885 | 14 | SNP | Nonsynonymous | P471S | rs56117859 | C/T |
| ATIC_35917 | 14 | SNP | Synonymous | G481G | | A/G |
| ATIC_35968 | 14 | SNP | Synonymous | T498T | | G/C |
| ATIC_35973 | 14 | SNP | Nonsynonymous | G500D | | G/A |
| ATIC_38338 | 15 | SNP | non-coding | IVS 15 + 53 | | −/+ deletion |
| | | | | | | GT |
| ATIC_38342 | 15 | SNP | non-coding | IVS 15 + 57 | | C/G |
| ATIC_38437 | 16 | SNP | non-coding | IVS 15 − 135 | rs4672768 | G/A |
| ATIC_38582 | 16 | SNP | Nonsynonymous | A557 | | C/T |
| ATIC_38627 | 16 | SNP | Nonsynonymous | I572T | | T/C |
| ATIC_38667 | 16 | SNP | Synonymous | T585T | | G/A |
| ATIC_38725 | 16 | SNP | non-coding | 3′UTR | | T/C |

TABLE C
MTHFS Variants

| GENE_position | Exon | Type | Function | Location | dB SNP id | Change |
|---|---|---|---|---|---|---|
| MTHFS_8636 | 2 | SNP | Non-coding | IVS1 − 39 | rs16971502 | C/T |
| MTHFS_8808 | 2 | SNP | Nonsynonymous | R84Q | | A/G |
| MTHFS_9012 | 2 | SNP | Nonsynonymous | V119L | | C/G |
| MTHFS_8957 | 2 | SNP | Non-coding | IVS2 + 21 | | A/G |
| MTHFS_8998 | 2 | SNP | Non-coding | IVS2 + 62 | | A/G |
| MTHFS_52560 | 3 | SNP | Non-coding | IVS2 − 27 | | C/T |
| MTHFS_52911 | 3 | SNP | Nonsynonymous | T202A | rs8923 | A/G |
| | | | | H280D | | A/G |
| MTHFS_52878 | 3 | SNP | Non-coding | 3′UTR | | G/T |
| MTHFS_52902 | 3 | SNP | Non-coding | 3′UTR | | Change |

TABLE D
MAT1A Variants

| GENE_position | Exon | Type | Function | Location | dB SNP id | Change |
|---|---|---|---|---|---|---|
| MAT1A_5045 | 2 | SNP | non-coding | IVS1 − 45 | | A/T |
| MAT1A_5081 | 2 | SNP | non-coding | IVS1 − 9 | rs10887721 | C/G |
| MAT1A_5181 | 2 | SNP | non-coding | IVS2 + 14 | | A/G |
| MAT1A_5233 | 2 | SNP | non-coding | 11152 + 66 | | A/G |
| MAT1A_6739 | 3 | SNP | Nonsynonymous | 190V | | A/G |
| MAT1A_6795 | 3 | SNP | non-coding | IVS3 + 32 | | G/T |
| MAT1A_9833 | 4 | SNP | non-coding | IVS3 − 54 | | C/T |
| MAT1A_10006 | 4 | SNP | non-coding | IVS4 + 7 | | C/T |
| MAT1A_10089 | 4 | SNP | non-coding | IVS4 + 90 | rs2282367 | C/T |
| MAT1A_10312 | 5 | SNP | non-coding | IVS4 − 51 | | C/T |
| MAT1A_10339 | 5 | SNP | non-coding | IVS4 − 24 | | A/G |
| MAT1A_10374 | 5 | SNP | Synonymous | F139F | | C/T |
| MAT1A_10383 | 5 | SNP | Synonymous | A142A | rs1143694 | C/T |
| MAT1A_10484 | 5 | SNP | Nonsynonymous | L176R | | G/T |
| MAT1A_10555 | 5 | SNP | non-coding | IVS5 + 49 | | A/C |
| MAT1A_14038 | 6 | SNP | non-coding | IVS5 − 47 | | A/G |
| MAT1A_14114 | 6 | SNP | Synonymous | G193G | | C/T |
| MAT1A_14177 | 6 | SNP | Synonymous | T214T | | A/G |
| MAT1A_15424 | 7 | SNP | non-coding | IVS6 − 56 | | A/C |
| MAT1A_15500 | 7 | SNP | Synonymous | G263G | | C/T |
| MAT1A_15581 | 7 | SNP | Synonymous | V290V | r 60582388 | A/G |
| MAT1A_15593 | 7 | SNP | Synonymous | A294A | rs59923268 | C/T |
| MAT1A_15596 | 7 | SNP | Synonymous | A295A | rs17851642 | A/T |
| MAT1A_15646 | 7 | SNP | Nonsynonymous | R312Q | | A/G |
| MAT1A_15706 | 7 | SNP | non-coding | IVS7 + 44 | | C/T |
| MAT1A_15715 | 7 | SNP | non-coding | IVS7 + 53 | | A/G |
| MAT1A_15730 | 7 | indel | non-coding | IVS7 + 68 | | −/+ deletion A |
| MAT1A_15758 | 7 | SNP | non-coding | IVS7 + 96 | | C/T |
| MAT1A_15760 | 7 | SNP | non-coding | IVS7 + 98 | rs10788545 | C/T |
| MAT1A_16133 | 8 | SNP | Synonymous | F353F | | C/T |
| MAT1A_16173 | 8 | SNP | non-coding | IVS8 + 14 | rs2994388 | C/T |
| MAT1A_16174 | 8 | SNP | non-coding | IVS8 + 15 | | A/G |
| MAT1A_16218 | 8 | SNP | non-coding | IVS8 + 59 | | A/T |
| MAT1A_16752 | 9 | SNP | non-coding | IVS8 − 44 | rs57820177 | C/T |
| MAT1A_16841 | 9 | SNP | Synonymous | Y377Y | rs57257983 | C/T |
| MAT1A_16965 | 9 | SNP | non-coding | 3′UTR | rs7087728 | C/T |
| MAT1A_16971 | 9 | SNP | non-coding | 3′UTR | | G/T |

TABLE E
MAT2A Variants

| GENE_position | Exon | Type | Function | Location | dB SNP id | Change |
|---|---|---|---|---|---|---|
| MAT2A_2871 | 2 | SNP | non-coding | IVS1 − 48 | | A/C |
| MAT2A_2873 | 2 | indel | non-coding | IVS1 − 50 | | −/+ |
| | | | | | | insertion |
| | | | | | | ATAC |
| MAT2A_2939 | 2 | SNP | Synonymous | Q360 | | A/G |
| MAT2A_3047 | 3 | SNP | non-coding | IVS2 − 48 | rs58507836 | A/G |
| MAT2A_3287 | 3 | SNP | non-coding | IVS3 + 70 | | A/G |
| MAT2A_3394 | 4 | SNP | non-coding | IVS3 − 79 | | C/T |
| MAT2A_3466 | 4 | SNP | non-coding | IVS3 − 7 | | C/G |
| MAT2A_3498 | 4 | SNP | Synonymous | V106V | | G/T |
| MAT2A_3617 | 4 | SNP | non-coding | IVS4 + 32 | rs62620249 | C/T |
| MAT2A_3650 | 5 | SNP | non-coding | IVS4 − 19 | | A/G |
| MAT2A_3704 | 5 | SNP | Synonymous | E147E | | A/G |
| MAT2A_3963 | 6 | SNP | non-coding | IVS5 − 32 | rs1078005 | A/G |
| MAT2A_4174 | 6 | SNP | Synonymous | H243H | | C/T |
| MAT2A_4428 | 7 | SNP | Synonymous | R264R | rs1078004 | C/G |
| MAT2A_4449 | 7 | SNP | Synonymous | Y271Y | | C/T |
| MAT2A_4476 | 7 | SNP | Synonymous | G280G | | C/T |
| MAT2A_4608 | 7 | SNP | non-coding | IVS7 + 21 | | C/G |
| MAT2A_4660 | 8 | SNP | non-coding | IVS7 − 81 | | C/G |
| MAT2A_4692 | 8 | SNP | non-coding | IVS7 − 49 | | A/G |
| MAT2A_4931 | 8 | indel | non-coding | IVS8 + 53 | | −/+ |
| | | | | | | insertion |
| | | | | | | GT |
| MAT2A_5313 | 9 | SNP | non-coding | IVS8 − 199 | | C/T |
| MAT2A_5460 | 9 | indel | non-coding | IVS8 − 54 | | −/+ |
| | | | | | | insertion T |
| MAT2A_5480 | 9 | SNP | non-coding | IVS8 − 33 | | C/T |

TABLE F
GART Variants

| GENE_position | Exon | Type | Function | Location | dB SNP id | Change |
|---|---|---|---|---|---|---|
| GART_3782 | 2 | SNP | non-coding | 5′UTR | | G/T |
| GART_3842 | 2 | SNP | Nonsynonymous | T16M | | C/T |
| GART_7745 | 3 | SNP | non-coding | IVS2 − 46 | | G/T |
| GART_7984 | 3 | SNP | non-coding | IVS3 + 98 | | C/T |
| GART_10720 | 5 | SNP | Nonsynonymous | A161G | rs35035222 | C/G |
| GART_10775 | 5 | SNP | non-coding | IVS5 + 9 | | A/G |
| GART_11521 | 6 | SNP | non-coding | IVS5 − 33 | | A/T |
| GART_11522 | 6 | SNP | non-coding | IVS5 − 32 | | A/T |
| GART_11541 | 6 | SNP | non-coding | IVS5 − 13 | | A/C |
| GART_12356 | 7 | SNP | non-coding | IVS7 + 4 | | C/T |
| GART_14200 | 8 | SNP | Synonymous | 12501 | | C/T |
| GART_14273 | 8 | SNP | non-coding | IVS8 + 12 | | C/T |
| GART_14282 | 8 | SNP | non-coding | IVS8 + 21 | | A/G |
| GART_14739 | 10 | SNP | non-coding | IVS9 − 37 | | A/C |
| GART_14781 | 10 | SNP | Synonymous | 13011 | | C/T |
| GART_18055 | 11 | SNP | non-coding | IVS10 − 55 | | C/T |
| GART_18064 | 11 | SNP | non-coding | IVS10 − 46 | | A/G |
| GART_18130 | 11 | SNP | Nonsynonymous | L3631 | | A/C |
| GART_18142 | 11 | SNP | Nonsynonymous | V367M | | A/G |
| GART_18197 | 11 | SNP | Nonsynonymous | R385K | | A/G |
| GART_18232 | 11 | SNP | Nonsynonymous | I397V | | A/G |
| GART_18304 | 11 | SNP | Nonsynonymous | V421I | rs60421747 | A/G |
| GART_18401 | 11 | SNP | non-coding | IVS11 + 60 | | A./T |
| GART_20794 | 12 | SNP | non-coding | IVS11 − 34 | rs2834234 | A/G |
| GART_20812 | 12 | SNP | non-coding | IVS11 − 16 | | A/G |
| GART_20825 | 12 | SNP | non-coding | IVS11 − 3 | | C/T |
| GART_20862 | 12 | SNP | Nonsynonymous | A445T | | A/G |
| GART_22073 | 13 | SNP | non-coding | IVS12 − 22 | rs2834232 | C/T |
| GART_22481 | 14 | SNP | non-coding | IVS13 − 67 | | A/G |
| GART_22521 | 14 | SNP | non-coding | IVS13 − 27 | rs2834232 | A/G |
| GART_22573 | 14 | SNP | Nonsynonymous | D510G | rs35927582 | A/G |
| GART_25425 | 15 | SNP | non-coding | IVS14 − 77 | | A/G |
| GART_25433 | 15 | SNP | non-coding | IVS14 − 69 | | C/G |
| GART_25601 | 15 | SNP | Nonsynonymous | H601R | | A/G |
| GART_25694 | 15 | SNP | Nonsynonymous | A632V | rs59920090 | C/T |
| GART_25720 | 15 | SNP | Nonsynonymous | P641A | rs34588874 | C/G |
| GART_25867 | 16 | SNP | non-coding | IVS15 − 102 | | C/T |
| GART_25912 | 16 | SNP | non-coding | IVS15 − 57 | | C/T |
| GART_25951 | 16 | SNP | non-coding | IVS15 − 18 | | C/T |
| GART_25956 | 16 | indel | non-coding | IVS15 − 13 | | −/+ |
| | | | | | | deletion |
| | | | | | | CT |
| GART_26127 | 16 | SNP | non-coding | IVS16 + 6 | | C/G |
| GART_26195 | 16 | SNP | non-coding | IVS16 + 74 | rs7281488 | A/G |
| GART_31619 | 17 | SNP | non-coding | IVS16 − 33 | | A/T |
| GART_31627 | 17 | SNP | non-coding | IVS16 − 25 | | A/G |
| GART_31641 | 17 | SNP | non-coding | IVS16 − 11 | rs8971 | A/G |
| GART_31799 | 17 | SNP | Nonsynonymous | D752G | | C/T |
| GART_31887 | 17 | SNP | non-coding | IVS17 + 29 | | C/T |
| GART_31902 | 17 | SNP | non-coding | IVS17 | | A/G |
| GART_31933 | 17 | SNP | non-coding | VS17 + 75 | | A/C |
| GART_33173 | 18 | SNP | non-coding | IVS17 − 17 | | A/G |
| GART_33264 | 18 | SNP | Nonsynonymous | L797M | | A/C |
| GART_33286 | 18 | SNP | Nonsynonymous | E804A | | A/C |
| GART_36963 | 19 | SNP | non-coding | IVS18 4 | | A/G |
| GART_36964 | 19 | SNP | non-coding | IVS18 − 42 | | A/T |
| GART_36967 | 19 | SNP | non-coding | IVS18 − 39 | rs2070390 | A/T |
| GART_37428 | 20 | SNP | Synonymous | Y868Y | | C/T |
| GART_37433 | 20 | SNP | Nonsynonymous | N870S | | A/G |
| GART_38709 | 21 | SNP | non-coding | IVS21 + 11 | rs2070388 | C/G |
| GART_38762 | 22 | SNP | non-coding | VS21 − 33 | | A/G |
| GART_38914 | 22 | SNP | Synonymous | A987A | | A/C |
| GART_38989 | 22 | SNP | non-coding | 3′ UTR | | C/G |

TABLE G
AHCY Genetic Variants

| | | | | | | | Poly | | | |
|---|---|---|---|---|---|---|---|---|---|---|
| GENE_Position | Exon | Type | Function | Location | dB SNP id | Change | Phen | SIFT | MAF | HWE |
| AHCY_996 | 1 | SNP | Non- | 5′UTR | | C/T | NA | NA | 0.001 | 1 |
| | | | coding | | | | | | | |
| AHCY_1014 | 1 | SNP | Non- | 5′UTR | rs57344541 | C/T | NA | NA | 0.003 | 0.997 |
| | | | coding | | | | | | | |
| AHCY_1017 | 1 | indel | Non- | 5′UTR | | insG | NA | NA | 0.001 | 1 |
| | | | coding | | | | | | | |
| AHCY_8673 | 1 | SNP | Non- | IVS1 − 61 | rs57865142 | C/T | NA | NA | 0.003 | 0.997 |
| | | | coding | | | | | | | |
| AHCY_8707 | 2 | SNP | Non- | IVS1 − 27 | | G/A | NA | NA | 0.001 | 1 |
| | | | coding | | | | | | | |
| AHCY_8817 | 2 | SNP | Nonsynonymous | R38W | rs13043752 | C/T | Probably | Affects | 0.019 | 0.929 |
| | | | | | | | Damaging | protein | | |
| | | | | | | | | function | | |
| AHCY_8931 | 2 | SNP | Non- | IVS2 + 7 | | C/G | NA | NA | 0.002 | 0.999 |
| | | | coding | | | | | | | |
| AHCY_8989 | 2 | SNP | Non- | IVS2 + 65 | | C/T | NA | NA | 0.001 | 1 |
| | | | coding | | | | | | | |
| AHCY_10139 | 2 | SNP | Non- | IVS2 − 24 | | G/A | NA | NA | 0.001 | 1 |
| | | | coding | | | | | | | |
| AHCY_10209 | 3 | SNP | Nonsynonymous | A89V | | C/T | Benign | Affects | 0.001 | 1 |
| | | | | | | | | protein | | |
| | | | | | | | | function | | |
| AHCY_10217 | 3 | SNP | Nonsynonymous | I92V | rs11552695 | A/G | Benign | Affects | 0.002 | 0.999 |
| | | | | | | | | protein | | |
| | | | | | | | | function | | |
| AHCY_10268 | 3 | SNP | Non- | IVS3 + 30 | | A/T | NA | NA | 0.001 | 1 |
| | | | coding | | | | | | | |
| AHCY_11765 | 3 | SNP | Non- | IVS3 − 47 | | G/T | NA | NA | 0.001 | 1 |
| | | | coding | | | | | | | |
| AHCY_11883 | 4 | SNP | Nonsynonymous | G123R | rs41301825 | G/A | Benign | Affects | 0.007 | 0.987 |
| | | | | | | | | protein | | |
| | | | | | | | | function | | |
| AHCY_11915 | 4 | SNP | Synonymous | G133G | | C/T | NA | NA | 0.001 | 1 |
| AHCY_11944 | 4 | SNP | Nonsynonymous | Y143C | | A/G | Benign | Tolerated | 0.001 | 1 |
| AHCY_12004 | 4 | SNP | Non- | IVS4 + 43 | | G/A | NA | NA | 0.001 | 1 |
| | | | coding | | | | | | | |
| AHCY_12713 | 4 | indel | Non- | IVS4 − 76 | | insC | NA | NA | 0.001 | 1 |
| | | | coding | | | | | | | |
| AHCY_12959 | 5 | SNP | Non- | IVS5 + 58 | | T/C | NA | NA | 0.003 | 0.998 |
| | | | coding | | | | | | | |
| AHCY_13645 | 5 | SNP | Non- | IVS6 − 37 | | C/G | NA | NA | 0.034 | 0.034 |
| | | | coding | | | | | | | |
| AHCY_13674 | 7 | indel | Non- | IVS6 − 8 | rs61664915 | delCT | NA | NA | 0.001 | 1 |
| | | | coding | | | | | | | |
| AHCY_13842 | 7 | SNP | Non- | IVS7 − 29 | rs57318446 | A/G | NA | NA | 0.001 | 1 |
| | | | coding | | | | | | | |
| AHCY_13886 | 8 | SNP | Nonsynonymous | M290I | | G/A | Possibly | Affects | 0.001 | 1 |
| | | | | | | | Damaging | protein | | |
| | | | | | | | | function | | |
| AHCY_18679 | 8 | SNP | Non- | IVS8 − 7 | | C/T | NA | NA | 0.037 | 0.037 |
| | | | coding | | | | | | | |
| AHCY_18692 | 9 | SNP | Nonsynonymous | R327W | | C/T | Benign | Affects | 0.001 | 1 |
| | | | | | | | | protein | | |
| | | | | | | | | function | | |
| AHCY_18721 | 9 | SNP | Synonymous | 1336I | | C/T | NA | NA | 0.001 | 1 |
| AHCY_23091 | 9 | SNP | Non- | IVS9 − 64 | rs17091693 | C/G | NA | NA | 0.037 | 0.037 |
| | | | coding | | | | | | | |
| AHCY_23141 | 10 | SNP | Non- | IVS9 − 14 | rs60143059 | T/C | NA | NA | 0.001 | 1 |
| | | | coding | | | | | | | |
| AHCY_23283 | 10 | SNP | Truncation | Y432− | | C/G | | | 0.001 | 1 |
| AHCY_23467 | 10 | SNP | Non- | 3′UTR | | C/T | NA | NA | 0.001 | 1 |
| | | | coding | | | | | | | |
| AHCY_23495 | 10 | SNP | Non- | 3′UTR | | G/A | NA | NA | 0.001 | 1 |
| | | | coding | | | | | | | |
| AHCY_23524 | 10 | SNP | Non- | 3′UTR | | A/C | NA | NA | 0.001 | 1 |
| | | | coding | | | | | | | |
| AHCY_23587 | 10 | SNP | Non- | 3′UTR | | T/G | NA | NA | 0.007 | 0.986 |
| | | | coding | | | | | | | |

TABLE H
AMT Genetic Variants

| | | | | | dB SNP | | | | | |
|---|---|---|---|---|---|---|---|---|---|---|
| GENE_Position | Exon | Type | Function | Location | id | Change | PolyPhen | SIFT | MAF | HWE |
| AMT_1129 | 1 | SNP | Non-coding | 5′UTR | | G/A | NA | NA | 0.002 | 0.999 |
| AMT_1435 | 2 | SNP | Nonsynonymous | R73C | | C/T | Possibly | Tolerated | 0.001 | 1 |
| | | | | | | | Damaging | | | |
| AMT_1449 | 2 | SNP | Synonymous | S77S | | G/A | NA | NA | 0.001 | 1 |
| AMT_3252 | 4 | SNP | Synonymous | L118L | | G/A | NA | NA | 0.002 | 0.999 |
| AMT_3381 | 4 | indel | Non-coding | IVS4 + 12 | | delCT | NA | NA | 0.002 | 0.999 |
| AMT_4255 | 6 | SNP | Nonsynonymous | E211K | | G/A | Benign | Affects | 0.016 | 0.959 |
| | | | | | | | | protein | | |
| | | | | | | | | function | | |
| AMT_4259 | 6 | SNP | Nonsynonymous | V212A | | T/C | Benign | Tolerated | 0.002 | 1 |
| AMT_4282 | 6 | indel | Frameshift | V220Fs | | insC | NA | NA | 0.002 | 1 |
| | | frame | | | | | | | | |
| | | shift | | | | | | | | |
| AMT_4484 | 7 | SNP | Nonsynonymous | P251R | | C/G | Benign | Tolerated | 0.002 | 1 |
| AMT_4599 | 7 | SNP | Synonymous | S289S | | T/C | NA | NA | 0.002 | 1 |
| AMT_5627 | 8 | SNP | Nonsynonymous | M300V | | A/G | Benign | Tolerated | 0.004 | 0.997 |
| AMT_5683 | 8 | SNP | Synonymous | R318R | rs11715915 | G/A | NA | NA | 0.247 | 0.256 |
| AMT_5851 | 9 | SNP | Non-coding | IVS8 − 11 | | T/C | NA | NA | 0.001 | 1 |

TABLE I
ATIC Genetic Variants

| GENE_position | Exon | Type | Function | Location | dB SNP id | Change | PolyPhen | SIFT | MAF | HWE |
|---|---|---|---|---|---|---|---|---|---|---|
| ATIC_1089 | 1 | SNP | non- | 5′UTR | rs28366034 | C/T | NA | NA | 0.174 | 0.931 |
| | | | coding | | | | | | | |
| ATIC_1100 | 1 | SNP | non- | 5′UTR | | C/T | NA | NA | 0.019 | 0.933 |
| | | | coding | | | | | | | |
| ATIC_1114 | 1 | SNP | non- | 5′UTR | | C/T | NA | NA | 0.003 | 0.999 |
| | | | coding | | | | | | | |
| ATIC_1116 | 1 | SNP | non- | 5′UTR | rs4535042 | T/G | NA | NA | 0.276 | 0.971 |
| | | | coding | | | | | | | |
| ATIC_1133 | 1 | SNP | non- | 5′UTR | rs28366035 | C/T/G | NA | NA | G = 0.011 | 0.469/0.957 |
| | | | coding | | | | | | T = 0.195 | |
| ATIC_1152 | 1 | SNP | non- | 5′UTR | rs11550205 | C/T | NA | NA | 0.01 | 0.983 |
| | | | coding | | | | | | | |
| ATIC_1160 | 1 | SNP | non- | 5′UTR | rs11550203 | C/T | NA | NA | 0.019 | 0.933 |
| | | | coding | | | | | | | |
| ATIC_1179 | 1 | SNP | Nonsynonymous | A2V | | C/T | Benign | Tolerated | 0.001 | 1 |
| ATIC_1244 | 1 | indel | non- | IVS1 + 50 | | | NA | NA | 0.001 | 1 |
| | | | coding | | | | | | | |
| ATIC_1270 | 1 | SNP | non- | IVS1 + 76 | | C/T | NA | NA | 0.007 | 0.991 |
| | | | coding | | | | | | | |
| ATIC_1288 | 1 | SNP | non- | IVS1 + 94 | | G/A | NA | NA | 0.009 | 0.983 |
| | | | coding | | | | | | | |
| ATIC_1301 | 1 | SNP | non- | IVS1 + 107 | | G/A | NA | NA | 0.001 | 1 |
| | | | coding | | | | | | | |
| ATIC_1380 | 2 | SNP | non- | IVS1 − 151 | | A/G | NA | NA | 0.015 | 0.959 |
| | | | coding | | | | | | | |
| ATIC_1396 | 2 | SNP | non- | IVS1 − 135 | | G/C | NA | NA | 0.001 | 1 |
| | | | coding | | | | | | | |
| ATIC_1453 | 2 | SNP | non- | IVS1 − 78 | | C/T | NA | NA | 0.007 | 0.992 |
| | | | coding | | | | | | | |
| ATIC_1506 | 2 | SNP | non- | IVS1 − 25 | | T/C | NA | NA | 0.001 | 1 |
| | | | coding | | | | | | | |
| ATIC_1689 | 2 | SNP | non- | IVS2 + 32 | | T/A | NA | NA | 0.012 | 0.972 |
| | | | coding | | | | | | | |
| ATIC_7227 | 3 | SNP | Nonsynonymous | G62R | | G/C | Possibly | Affects | 0.001 | 1 |
| | | | | | | | Damaging | protein | | |
| | | | | | | | | function | | |
| ATIC_7232 | 3 | indel | Nonsynonymous | G63fs | | —/G | Frameshift | Frameshift | 0.001 | 1 |
| ATIC_7388 | 3 | SNP | non- | IVS3 + 121 | | T/A | NA | NA | 0.003 | 0.997 |
| | | | coding | | | | | | | |
| ATIC_8756 | 4 | SNP | Nonsynonymous | N94S | | A/G | Benign | Tolerated | 0.003 | 0.997 |
| ATIC_8793 | 4 | SNP | non- | IVS4 + 28 | rs16853782 | A/G | NA | NA | 0.28 | 0.999 |
| | | | coding | | | | | | | |
| ATIC_8808 | 4 | SNP | non- | IVS4 + 43 | | G/A | NA | NA | 0.002 | 0.999 |
| | | | coding | | | | | | | |
| ATIC_14099 | 5 | SNP | non- | IVS4 − 176 | | C/T | NA | NA | 0.009 | 0.984 |
| | | | coding | | | | | | | |
| ATIC_14136 | 5 | SNP | non- | IVS4 − 139 | rs3772077 | A/G | NA | NA | 0.295 | 0.55 |
| | | | coding | | | | | | | |
| ATIC_14140 | 5 | SNP | non- | IVS4 − 135 | | C/A | NA | NA | 0.002 | 0.999 |
| | | | coding | | | | | | | |
| ATIC_14144 | 5 | SNP | non- | IVS4 − 131 | | C/T | NA | NA | 0.001 | 1 |
| | | | coding | | | | | | | |
| ATIC_14156 | 5 | SNP | non- | IVS4 − 119 | rs3772078 | A/G | NA | NA | 0.288 | 0.61 |
| | | | coding | | | | | | | |
| ATIC_14183 | 5 | SNP | non- | IVS4 − 92 | | C/T | NA | NA | 0.319 | 1 |
| | | | coding | | | | | | | |
| ATIC_14229 | 5 | SNP | non- | IVS4 − 46 | | A/G | NA | NA | 0.005 | 0.995 |
| | | | coding | | | | | | | |
| ATIC_14238 | 5 | SNP | non- | IVS4 − 37 | | C/T | NA | NA | 0.004 | 0.997 |
| | | | coding | | | | | | | |
| ATIC_14245 | 5 | SNP | non- | IVS4 − 30 | | A/C | NA | NA | 0.001 | 1 |
| | | | coding | | | | | | | |
| ATIC_14260 | 5 | SNP | non- | IVS4 − 15 | | G/T | NA | NA | 0.001 | 1 |
| | | | coding | | | | | | | |
| ATIC_14331 | 5 | SNP | Nonsynonymous | T116S | rs2372536 | C/G | Benign | Tolerated | 0.295 | 0.956 |
| ATIC_14489 | 5 | SNP | non- | IVS5 + 126 | | G/A | NA | NA | 0.001 | 1 |
| | | | coding | | | | | | | |
| ATIC_14965 | 6 | SNP | non- | IVS5 − 56 | rs7563206 | C/T | NA | NA | 0.386 | 0.638 |
| | | | coding | | | | | | | |
| ATIC_14970 | 6 | SNP | non- | IVS5 − 51 | | C/T | NA | NA | 0.005 | 0.995 |
| | | | coding | | | | | | | |
| ATIC_15003 | 6 | SNP | non- | IVS5 − 18 | | G/A | NA | NA | 0.005 | 0.995 |
| | | | coding | | | | | | | |
| ATIC_15040 | 6 | SNP | Synonymous | R133R | | A/G | NA | NA | 0.001 | 1 |
| ATIC_15043 | 6 | SNP | Synonymous | A134A | | T/C | NA | NA | 0.001 | 1 |
| ATIC_15149 | 6 | SNP | Nonsynonymous | T170A | | A/G | Benign | Tolerated | 0.001 | 1 |
| ATIC_15240 | 6 | SNP | non- | IVS6 + 68 | | A/G | NA | NA | 0.001 | 1 |
| | | | coding | | | | | | | |
| ATIC_15826 | 7 | SNP | non- | IVS6 − 30 | rs6751557 | C/T | NA | NA | 0.395 | 0.749 |
| | | | coding | | | | | | | |
| ATIC_15844 | 7 | SNP | non- | IVS6 − 12 | | C/T | NA | NA | 0.285 | 0.777 |
| | | | coding | | | | | | | |
| ATIC_16063 | 7 | SNP | non- | IVS7 + 51 | | G/A | NA | NA | 0.019 | 0 |
| | | | coding | | | | | | | |
| ATIC_21363 | 8 | SNP | non- | IVS7 − 53 | | A/G | NA | NA | 0.006 | 0.993 |
| | | | coding | | | | | | | |
| ATIC_21372 | 8 | SNP | non- | IVS7 − 44 | | T/G | NA | NA | 0.006 | 0.993 |
| | | | coding | | | | | | | |
| ATIC_21400 | 8 | SNP | non- | IVS7 − 16 | | A/G | NA | NA | 0.002 | 0.999 |
| | | | coding | | | | | | | |
| ATIC_21521 | 8 | indel | Nonsynonymous | F265fs | | T/— | Frameshift | Frameshift | 0.001 | 1 |
| ATIC_21611 | 8 | SNP | non- | IVS8 + 70 | | T/A | NA | NA | 0.001 | 1 |
| | | | coding | | | | | | | |
| ATIC_22187 | 9 | SNP | non- | IVS8 − 197 | | G/A | NA | NA | 0.015 | 0.006 |
| | | | coding | | | | | | | |
| ATIC_22273 | 9 | SNP | non- | IVS8 − 111 | | A/G | NA | NA | 0.001 | 1 |
| | | | coding | | | | | | | |
| ATIC_22282 | 9 | indel | non- | IVS8 − 103 | | —/A | NA | NA | 0.007 | 0.991 |
| | | | coding | | | | | | | |
| ATIC_22283 | 9 | SNP | non- | IVS8 − 102 | rs12995526 | C/T | NA | NA | 0.425 | 0.099 |
| | | | coding | | | | | | | |
| ATIC_22291 | 9 | SNP | non- | IVS8 − 94 | | G/A | NA | NA | 0.061 | 0.466 |
| | | | coding | | | | | | | |
| ATIC_22342 | 9 | SNP | non- | IVS8 − 43 | | A/G | NA | NA | 0.001 | 1 |
| | | | coding | | | | | | | |
| ATIC_22361 | 9 | SNP | non- | IVS8 − 24 | rs10179873 | A/G | NA | NA | 0.165 | 1 |
| | | | coding | | | | | | | |
| ATIC_22512 | 9 | SNP | non- | IVS9 + 20 | | T/G | NA | NA | 0.001 | 1 |
| | | | coding | | | | | | | |
| ATIC_22519 | 9 | SNP | non- | IVS9 + 27 | | G/T | NA | NA | 0.001 | 1 |
| | | | coding | | | | | | | |
| ATIC_22538 | 9 | SNP | non- | IVS9 + 46 | | A/G | NA | NA | 0.007 | 0.991 |
| | | | coding | | | | | | | |
| ATIC_22564 | 9 | indel | non- | IVS9 + 72 | | GGA/— | NA | NA | 0.001 | 1 |
| | | | coding | | | | | | | |
| ATIC_22589 | 9 | SNP | non- | IVS9 + 97 | | G/T | NA | NA | 0.002 | 0.999 |
| | | | coding | | | | | | | |
| ATIC_22686 | 9 | SNP | non- | IVS9 + 194 | rs10932606 | C/T | NA | NA | 0.2 | 0.339 |
| | | | coding | | | | | | | |
| ATIC_22737 | 9 | SNP | non- | IVS9 + 245 | | A/G | NA | NA | 0.007 | 0 |
| | | | coding | | | | | | | |
| ATIC_24992 | 11 | indel | non- | IVS10 − 79 | | —/G | NA | NA | 0.001 | 1 |
| | | | coding | | | | | | | |
| ATIC_25009 | 11 | SNP | non- | IVS10 − 62 | | A/G | NA | NA | 0.001 | 1 |
| | | | coding | | | | | | | |
| ATIC_25220 | 11 | SNP | non- | IVS11 + 60 | rs13002576 | G/C | NA | NA | 0.416 | 0.283 |
| | | | coding | | | | | | | |
| ATIC_27609 | 12 | SNP | non- | IVS11 − | rs16853823 | A/G | NA | NA | 0.294 | 0.652 |
| | | | coding | 206 | | | | | | |
| ATIC_27739 | 12 | SNP | non- | IVS11 − 76 | rs6721444 | C/A | NA | NA | 0.017 | 0 |
| | | | coding | | | | | | | |
| ATIC_27757 | 12 | SNP | non- | IVS11 − 58 | | A/G | NA | NA | 0.001 | 1 |
| | | | coding | | | | | | | |
| ATIC_27855 | 12 | SNP | Nonsynonymous | T380I | | C/T | Benign | Tolerated | 0.001 | 1 |
| ATIC_27985 | 12 | SNP | non- | IVS12 + 42 | | T/C | NA | NA | 0.004 | 0.995 |
| | | | coding | | | | | | | |
| ATIC_28015 | 12 | SNP | non- | IVS12 + 72 | | A/G | NA | NA | 0.001 | 1 |
| | | | coding | | | | | | | |
| ATIC_33785 | 13 | SNP | non- | IVS12 − 30 | rs13010249 | A/G | NA | NA | 0.304 | 0.478 |
| | | | coding | | | | | | | |
| ATIC_33901 | 13 | SNP | Synonymous | N438N | | C/T | NA | NA | 0.001 | 1 |
| ATIC_33919 | 13 | SNP | non- | IVS13 + 12 | | G/A | NA | NA | 0.001 | 1 |
| | | | coding | | | | | | | |
| ATIC_33920 | 13 | SNP | non- | IVS13 + 13 | | T/C | NA | NA | 0.001 | 1 |
| | | | coding | | | | | | | |
| ATIC_33933 | 13 | SNP | non- | IVS13 + 26 | | C/T | NA | NA | 0.001 | 1 |
| | | | coding | | | | | | | |
| ATIC_35723 | 14 | SNP | non- | IVS13 − 72 | | G/A | NA | NA | 0.003 | 0.997 |
| | | | coding | | | | | | | |
| ATIC_35737 | 14 | SNP | non- | IVS13 − 58 | | C/A | NA | NA | 0.001 | 1 |
| | | | coding | | | | | | | |
| ATIC_35742 | 14 | SNP | non- | IVS13 − 53 | | G/C | NA | NA | 0.001 | 1 |
| | | | coding | | | | | | | |
| ATIC_35840 | 14 | SNP | Nonsynonymous | R456S | | C/A | Probably | Affects | 0.001 | 1 |
| | | | | | | | Damaging | protein | | |
| | | | | | | | | function | | |
| ATIC_35885 | 14 | SNP | Nonsynonymous | P471S | rs56117859 | C/T | Benign | Affects | 0.008 | 0 |
| | | | | | | | | protein | | |
| | | | | | | | | function | | |
| ATIC_35917 | 14 | SNP | Synonymous | G481G | | A/G | NA | NA | 0.014 | 0.953 |
| ATIC_35968 | 14 | SNP | Synonymous | T498T | | C/G | NA | NA | 0.008 | 0.986 |
| ATIC_35973 | 14 | SNP | Nonsynonymous | G500D | | G/A | Possibly | Tolerated | 0.002 | 0.999 |
| | | | | | | | Damaging | | | |
| ATIC_38338 | 15 | indel | non- | IVS15 + 53 | | GT/— | NA | NA | 0.001 | 1 |
| | | | coding | | | | | | | |
| ATIC_38342 | 15 | SNP | non- | IVS15 + 57 | | C/G | NA | NA | 0.003 | 0.997 |
| | | | coding | | | | | | | |
| ATIC_38437 | 16 | SNP | non- | IVS15 − | rs4672768 | G/A | NA | NA | 0.307 | 0.413 |
| | | | coding | 135 | | | | | | |
| ATIC_38582 | 16 | SNP | Nonsynonymous | A557V | | C/T | Benign | Affects | 0.003 | 0.997 |
| | | | | | | | | protein | | |
| | | | | | | | | function | | |
| ATIC_38627 | 16 | SNP | Nonsynonymous | I572T | | T/C | Possibly | Tolerated | 0.003 | 0.997 |
| | | | | | | | Damaging | | | |
| ATIC_38667 | 16 | SNP | Synonymous | T585T | | G/A | NA | NA | 0.001 | 1 |
| ATIC_38725 | 16 | SNP | non- | 3′UTR | | T/C | NA | NA | 0.001 | 1 |
| | | | coding | | | | | | | |

TABLE J
CBS Genetic Variants

| | | | | | | | Poly | | | |
|---|---|---|---|---|---|---|---|---|---|---|
| GENE_position | Exon | Type | Function | Location | dB SNP id | Change | Phen | SIFT | MAF | HWE |
| CBS_4569 | 3 | SNP | non-coding | IVS2 − 72 | | C/T | NA | NA | 0.001 | 1 |
| CBS_4605 | 3 | SNP | non-coding | IVS2 − 36 | | G/A | NA | NA | 0.005 | 0.993 |
| CBS_4700 | 3 | SNP | Nonsynonymous | R18C | | C/T | Benign | Tolerated | 0.002 | 0.999 |
| CBS_8211 | 4 | SNP | non-coding | IVS3 − 16 | | G/T | NA | NA | 0.001 | 1 |
| CBS_8232 | 4 | SNP | Nonsynonymous | K72I | | A/T | Benign | Tolerated | 0.001 | 1 |
| CBS_8238 | 4 | SNP | Nonsynonymous | P74L | | C/T | Possibly | Tolerated | 0.002 | 0.999 |
| | | | | | | | Damaging | | | |
| CBS_8321 | 4 | SNP | Nonsynonymous | K102Q | rs34040148 | A/C | Benign | Tolerated | 0.006 | 0.99 |
| CBS_10419 | 5 | SNP | non-coding | IVS4 − 46 | | G/C | NA | NA | 0.009 | 0.984 |
| CBS_10628 | 5 | SNP | non-coding | IVS5 + 29 | rs234708 | A/G | NA | NA | 0.291 | 0.164 |
| CBS_10669 | 5 | SNP | non-coding | IVS5 + 70 | | C/T | NA | NA | 0.005 | 0.995 |
| CBS_10672 | 5 | SNP | non-coding | IVS5 + 73 | | G/T | NA | NA | 0.001 | 1 |
| CBS_10674 | 5 | SNP | non-coding | IVS5 + 75 | rs7279359 | G/A | NA | NA | 0.037 | 0.99 |
| CBS_10681 | 5 | SNP | non-coding | IVS5 + 82 | rs234707 | C/A | NA | NA | 0.125 | 0.621 |
| CBS_12958 | 9 | SNP | non-coding | IVS9 + 16 | | G/A | NA | NA | 0.001 | 1 |
| CBS_12965 | 9 | SNP | non-coding | IVS9 + 23 | | A/G | NA | NA | 0.077 | 0.114 |
| CBS_13704 | 10 | SNP | non-coding | IVS9 − 60 | rs12329764 | C/T | NA | NA | 0.011 | 0 |
| CBS_13720 | 10 | SNP | non-coding | IVS9 − 44 | rs9978863 | G/A | NA | NA | 0.001 | 1 |
| CBS_13768 | 10 | indel | non-coding | IVS9 − 64 | | insCTGG | NA | NA | 0.067 | 0.017 |
| | | | | | | GGTGG | | | | |
| | | | | | | ATCAT | | | | |
| | | | | | | CCAGG | | | | |
| | | | | | | TGGGG | | | | |
| | | | | | | CTTTTG | | | | |
| | | | | | | CTGGG | | | | |
| | | | | | | CTTGA | | | | |
| | | | | | | GCCCT | | | | |
| | | | | | | GAAGC | | | | |
| | | | | | | CGCGC | | | | |
| | | | | | | CCTCT | | | | |
| | | | | | | GCAGA | | | | |
| | | | | | | TCA | | | | |
| CBS_13820 | 10 | SNP | non-coding | IVS9 − 12 | | C/T | NA | NA | 0.016 | 0.94 |
| CBS_13942 | 10 | SNP | Synonymous | T313T | rs2228298 | G/A | NA | NA | 0.001 | 1 |
| CBS_13965 | 10 | SNP | non-coding | IVS10 + 8 | | G/A | NA | NA | 0.002 | 0.999 |
| CBS_13990 | 10 | SNP | non-coding | IVS10 + 33 | rs59521601 | G/A | NA | NA | 0.033 | 0.098 |
| CBS_14004 | 10 | SNP | non-coding | IVS10 + 47 | rs57282132 | C/T | NA | NA | 0.001 | 1 |
| CBS_14010 | 10 | SNP | non-coding | IVS10 + 53 | | C/T | NA | NA | 0.001 | 1 |
| CBS_14084 | 10 | SNP | non-coding | IVS10 + 127 | rs1789953 | G/A | NA | NA | 0.22 | 0.01 |
| CBS_14114 | 10 | SNP | non-coding | IVS10 + 157 | | G/A | NA | NA | 0.001 | 1 |
| CBS_14432 | 11 | SNP | non-coding | IVS10 − 83 | | G/A | NA | NA | 0.001 | 1 |
| CBS_14467 | 11 | SNP | non-coding | IVS10 − 48 | | C/T | NA | NA | 0.004 | 0.995 |
| CBS_16304 | 12 | SNP | non-coding | IVS11 − 60 | | C/G | NA | NA | 0.001 | 1 |
| CBS_16321 | 12 | SNP | non-coding | IVS11 − 43 | | G/A | NA | NA | 0.002 | 0.999 |
| CBS_16324 | 12 | SNP | non-coding | IVS11 − 40 | | C/T | NA | NA | 0.001 | 1 |
| CBS_16337 | 12 | SNP | non-coding | IVS11 − 27 | | G/A | NA | NA | 0.001 | 1 |
| CBS_16338 | 12 | SNP | non-coding | IVS11 − 26 | | C/T | NA | NA | 0.001 | 1 |
| CBS_16339 | 12 | SNP | non-coding | IVS11 − 25 | | G/A | NA | NA | 0.001 | 1 |
| CBS_16364 | 12 | SNP | Nonsynonymous | G347D | | G/A | Probably | Affects | 0.001 | 1 |
| | | | | | | | Damaging | protein | | |
| | | | | | | | | function | | |
| CBS_16368 | 12 | SNP | Synonymous | G348G | | C/T | NA | NA | 0.001 | 1 |
| CBS_16377 | 12 | SNP | Synonymous | G351G | | C/T | NA | NA | 0.002 | 0.999 |
| CBS_16380 | 12 | SNP | Synonymous | S352S | | C/T | NA | NA | 0.003 | 0.997 |
| CBS_16383 | 12 | SNP | Synonymous | T353T | rs61735859 | G/A | NA | NA | 0.003 | 0 |
| CBS_16388 | 12 | SNP | Nonsynonymous | A355E | | C/A | Probably | Tolerated | 0.001 | 1 |
| | | | | | | | Damaging | | | |
| CBS_16393 | 12 | SNP | Nonsynonymous | A357T | | G/A | Probably | Tolerated | 0.001 | 1 |
| | | | | | | | Damaging | | | |
| CBS_16402 | 12 | SNP | Nonsynonymous | A360T | | G/A | Benign | Tolerated | 0.002 | 0.999 |
| CBS_16404 | 12 | SNP | Synonymous | A360A | rs1801181 | C/T | NA | NA | 0.294 | 0 |
| CBS_16405 | 12 | SNP | Nonsynonymous | A361T | | G/A | Probably | Affects | 0.001 | 1 |
| | | | | | | | Damaging | protein | | |
| | | | | | | | | function | | |
| CBS_16406 | 12 | SNP | Nonsynonymous | A361V | | C/T | Benign | Tolerated | 0.002 | 0.999 |
| CBS_16417 | 12 | SNP | Truncation | Q365− | | C/T | Stop | Stop | 0.002 | 0.999 |
| CBS_16425 | 12 | SNP | Synonymous | G367G | | C/T | NA | NA | 0.001 | 1 |
| CBS_16429 | 12 | SNP | Nonsynonymous | R369C | | C/T | Probably | Affects | 0.001 | 1 |
| | | | | | | | Damaging | protein | | |
| | | | | | | | | function | | |
| CBS_16476 | 12 | SNP | non-coding | IVS12 + 7 | | C/T | NA | NA | 0.005 | 0 |
| CBS_18602 | 15 | SNP | non-coding | IVS14 − 55 | | C/T | NA | NA | 0.004 | 0.996 |
| CBS_18627 | 15 | SNP | non-coding | IVS14 − 30 | rs6586281 | C/T | NA | NA | 0.161 | 0.819 |
| CBS_18643 | 15 | SNP | non-coding | IVS14 − 14 | | C/T | NA | NA | 0.002 | 0.999 |
| CBS_19978 | 16 | SNP | non-coding | IVS15 − 45 | | C/T | NA | NA | 0.001 | 1 |
| CBS_19981 | 16 | SNP | non-coding | IVS15 − 42 | | G/T | NA | NA | 0.001 | 1 |
| CBS_19987 | 16 | SNP | non-coding | IVS15 − 36 | rs1005585 | A/G | NA | NA | 0.058 | 0.129 |
| CBS_20039 | 16 | SNP | Nonsynonymous | T495M | | C/T | Possibly | Tolerated | 0.001 | 1 |
| | | | | | | | Damaging | | | |
| CBS_20049 | 16 | SNP | Synonymous | R498R | | G/A | NA | NA | 0.001 | 1 |
| CBS_20067 | 16 | SNP | Synonymous | E504E | | G/A | NA | NA | 0.001 | 1 |
| CBS_20191 | 16 | SNP | non-coding | IVS16 + 84 | | C/T | NA | NA | 0.001 | 1 |
| CBS_22825 | 17 | SNP | non-coding | IVS16 − | | A/G | NA | NA | 0.001 | 1 |
| | | | | 102 | | | | | | |
| CBS_22867 | 17 | SNP | non-coding | IVS16 − 60 | | G/A | NA | NA | 0.001 | 1 |
| CBS_22879 | 17 | SNP | non-coding | IVS16 − 48 | | C/T | NA | NA | 0.001 | 1 |
| CBS_22899 | 17 | SNP | non-coding | IVS16 − 28 | | C/G | NA | NA | 0.001 | 1 |
| CBS_23017 | 17 | SNP | Nonsynonymous | R548Q | | G/A | Benign | Tolerated | 0.002 | 0.999 |
| CBS_23040 | 17 | SNP | non-coding | 3′UTR | rs9978104 | C/A | NA | NA | 0.083 | 0.011 |
| CBS_23048 | 17 | SNP | non-coding | 3′UTR | | G/A | NA | NA | 0.001 | 1 |
| CBS_23110 | 17 | SNP | non-coding | 3′UTR | | C/T | NA | NA | 0.001 | 1 |
| CBS_23111 | 17 | SNP | non-coding | 3′UTR | | G/A | NA | NA | 0.007 | 0 |

TABLE K
CTH Genetic Variants

| | | | | | | | Poly | | | |
|---|---|---|---|---|---|---|---|---|---|---|
| GENE_position | Exon | Type | Function | Location | dB SNP id | Change | Phen | SIFT | MAF | HWE |
| CTH_1273 | 1 | SNP | Synonymous | L43L | rs61735624 | G/A | NA | NA | 0.001 | 1 |
| CTH_5632 | 2 | SNP | non-coding | IVS1 − 53 | rs41313347 | C/A | NA | NA | 0.009 | 0.985 |
| CTH_5667 | 2 | SNP | non-coding | IVS1 − 18 | | C/T | NA | NA | 0.007 | 0.989 |
| CTH_5716 | 2 | SNP | Nonsynonymous | T67I | rs28941785 | C/T | Probably | Affects | 0.009 | 0 |
| | | | | | | | Damaging | protein | | |
| | | | | | | | | function | | |
| CTH_5723 | 2 | SNP | Synonymous | N69N | | T/C | NA | NA | 0.001 | 1 |
| CTH_5824 | 2 | SNP | non-coding | IVS2 + 58 | | T/C | NA | NA | 0.001 | 1 |
| CTH_7632 | 3 | SNP | non-coding | IVS2 − 34 | | T/G | NA | NA | 0.001 | 1 |
| CTH_7886 | 3 | SNP | non-coding | IVS3 + 125 | rs1145920 | G/A | NA | NA | 0.19 | 0.498 |
| CTH_11229 | 4 | SNP | non-coding | IVS3 − 66 | rs6413471 | A/C | NA | NA | 0.078 | 0.712 |
| CTH_11243 | 4 | SNP | non-coding | IVS3 − 52 | | T/C | NA | NA | 0.001 | 1 |
| CTH_14036 | 5 | SNP | Nonsynonymous | T160K | | C/A | Probably | Affects | 0.002 | 1 |
| | | | | | | | Damaging | protein | | |
| | | | | | | | | function | | |
| CTH_14053 | 5 | SNP | Nonsynonymous | V166M | | G/A | Benign | Tolerated | 0.002 | 1 |
| CTH_14264 | 5 | SNP | non-coding | IVS5 + 119 | | A/G | NA | NA | 0.002 | 1 |
| CTH_14304 | 5 | SNP | non-coding | IVS5 + 159 | | C/T | NA | NA | 0.002 | 1 |
| CTH_14358 | 5 | SNP | non-coding | IVS5 + 213 | | T/G | NA | NA | 0.002 | 1 |
| CTH_19447 | 6 | SNP | non-coding | IVS5 − 76 | | A/G | NA | NA | 0.002 | 0.999 |
| CTH_20017 | 7 | SNP | non-coding | IVS6 − 29 | | T/C | NA | NA | 0.084 | 0 |
| CTH_20031 | 7 | SNP | non-coding | IVS6 − 15 | | G/C | NA | NA | 0.004 | 0.997 |
| CTH_20038 | 7 | SNP | non-coding | IVS6 − 8 | | G/C | NA | NA | 0.001 | 1 |
| CTH_20090 | 7 | SNP | Nonsynonymous | S231R | | A/C | Benign | Tolerated | 0.001 | 1 |
| CTH_21783 | 8 | SNP | non-coding | IVS7 − 29 | | A/G | NA | NA | 0.004 | 0.997 |
| CTH_23502 | 9 | SNP | non-coding | IVS8 − 55 | | C/T or G | NA | NA | 0.002 | 1 |
| CTH_23509 | 9 | indel | non-coding | IVS8 − 49 | | insA | NA | NA | 0.001 | 1 |
| CTH_23704 | 9 | SNP | non-coding | IVS9 + 25 | | T/C | NA | NA | 0.002 | 0.999 |
| CTH_24825 | 10 | SNP | non-coding | IVS9 − 30 | | A/T | NA | NA | 0.001 | 1 |
| CTH_24892 | 10 | SNP | Nonsynonymous | S346T | | C/T | Benign | Affects | 0.001 | 1 |
| | | | | | | | | protein | | |
| | | | | | | | | function | | |
| CTH_28520 | 11 | SNP | Nonsynonymous | D385E | | C/A | Possibly | Affects | 0.003 | 0.997 |
| | | | | | | | Damaging | protein | | |
| | | | | | | | | function | | |
| CTH_28628 | 11 | SNP | non-coding | IVS11 + 72 | | A/G | NA | NA | 0.002 | 0.999 |
| CTH_28737 | 12 | SNP | non-coding | IVS11 − 94 | | G/C | NA | NA | 0.001 | 1 |
| CTH_28789 | 12 | SNP | non-coding | IVS11 − 42 | | T/C | NA | NA | 0.001 | 1 |
| CTH_28846 | 12 | SNP | Nonsynonymous | S403G | rs1021737 | G/T | NA | Tolerated | 0.336 | 0.199 |

TABLE L
DHFR Genetic Variants

| | | | | | | | Poly | | | |
|---|---|---|---|---|---|---|---|---|---|---|
| GENE_position | Exon | Type | Function | Location | dB SNP id | Change | Phen | SIFT | MAF | HWE |
| DHFR_6339 | 3 | SNP | non-coding | IVS2 − 149 | | A/T | NA | NA | 0.001 | 1 |
| DHFR_6461 | 3 | SNP | non-coding | IVS2 − 27 | | A/G | NA | NA | 0.001 | 1 |
| DHFR_6538 | 3 | SNP | Nonsynonymous | E63Q | | G/C | Benign | Tolerated | 0.001 | 1 |
| DHFR_6661 | 3 | SNP | non-coding | IVS3 + 68 | rs10072026 | A/G | NA | NA | 0.116 | 0.272 |
| DHFR_17868 | 4 | SNP | non-coding | IVS3 − 105 | rs1677697 | A/G | NA | NA | 0.07 | 0 |
| DHFR_17874 | 4 | SNP | non-coding | IVS3 − 99 | | G/A | NA | NA | 0.029 | 0.851 |
| DHFR_18075 | 4 | SNP | Synonymous | I115I | | A/T | NA | NA | 0.001 | 1 |
| DHFR_18148 | 4 | indel | non-coding | IVS4 + 45 | | insTTTC | NA | NA | 0.165 | 0.168 |
| DHFR_18199 | 4 | SNP | non-coding | IVS4 + 96 | | T/G | NA | NA | 0.004 | 0.998 |
| DHFR_18229 | 4 | SNP | non-coding | IVS4 + 126 | rs1643661 | G/A | NA | NA | ?? | 0 |
| DHFR_22042 | 5 | SNP | Nonsynonymous | M140L | | A/C | Benign | Tolerated | 0.001 | 1 |
| DHFR_26721 | 6 | SNP | non-coding | IVS5 − 100 | rs3797876 | C/T | NA | NA | 0.004 | 0.996 |
| DHFR_26822 | 6 | SNP | Nonsynonymous | Y163H | | T/C | Benign | Tolerated | 0.001 | 1 |
| DHFR_27014 | 6 | SNP | non-coding | 3′UTR | rs7387 | A/T | NA | NA | 0.174 | 0.205 |

TABLE M
FPGS Genetic Variants

| | | | | | | | Poly | | | |
|---|---|---|---|---|---|---|---|---|---|---|
| GENE_position | Exon | Type | Function | Location | dB SNP id | Change | Phen | SIFT | MAF | HWE |
| FPGS_2386 | 2 | SNP | non-coding | IVS1 − 25 | rs7856096 | A/G | NA | NA | 0.03 | 0.604 |
| FPGS_2420 | 2 | SNP | Nonsynonymous | R50C | | C/T | Benign | Tolerated | 0.001 | 1 |
| FPGS_2515 | 2 | SNP | Synonymous | L81L | rs34330923 | G/A | NA | NA | 0.005 | 0.993 |
| FPGS_2525 | 2 | SNP | Nonsynonymous | R85W | rs41306702 | C/T | Probably | Affects | 0.003 | 0.997 |
| | | | | | | | Damaging | protein | | |
| | | | | | | | | function | | |
| FPGS_2770 | 4 | SNP | Synonymous | T110T | | C/T | NA | NA | 0.002 | 0.999 |
| FPGS_5042 | 5 | SNP | non-coding | IVS4 − 57 | | G/A | NA | NA | 0.001 | 1 |
| FPGS_5218 | 5 | SNP | non-coding | IVS5 + 5 | | C/T | NA | NA | 0.001 | 1 |
| FPGS_5507 | 7 | SNP | non-coding | IVS6 − 40 | | C/T | NA | NA | 0.001 | 1 |
| FPGS_5614 | 7 | SNP | non-coding | IVS7 + 6 | | C/T | NA | NA | 0.001 | 1 |
| FPGS_5659 | 7 | SNP | non-coding | IVS7 + 51 | | C/T | NA | NA | 0.01 | 0.978 |
| FPGS_5667 | 8 | SNP | non-coding | IVS7 − 45 | | C/T | NA | NA | 0.001 | 1 |
| FPGS_5680 | 8 | SNP | non-coding | IVS7 − 32 | | G/A | NA | NA | 0.001 | 1 |
| FPGS_6456 | 9 | SNP | non-coding | IVS9 + 19 | | G/A | NA | NA | 0.008 | 0.983 |
| FPGS_6471 | 9 | SNP | non-coding | IVS9 + 34 | | G/A | NA | NA | 0.001 | 1 |
| FPGS_6485 | 9 | SNP | non-coding | IVS9 + 48 | rs41307463 | C/T | NA | NA | 0.012 | 0.967 |
| FPGS_6635 | 10 | SNP | non-coding | IVS9 − 49 | | C/T | NA | NA | 0.001 | 1 |
| FPGS_6639 | 10 | SNP | non-coding | IVS9 − 45 | | G/A | NA | NA | 0.008 | 0.983 |
| FPGS_6719 | 10 | SNP | Synonymous | L286L | | C/T | NA | NA | 0.001 | 1 |
| FPGS_6726 | 10 | SNP | Nonsynonymous | G289W | | G/A | | | 0.001 | 1 |
| FPGS_6951 | 11 | SNP | Synonymous | R332R | | G/A | NA | NA | 0.001 | 1 |
| FPGS_6979 | 11 | SNP | Nonsynonymous | A342S | | G/T | Benign | Tolerated | 0.001 | 1 |
| FPGS_6980 | 11 | SNP | Nonsynonymous | A342V | | C/T | Benign | Tolerated | 0.001 | 1 |
| FPGS_9195 | 14 | SNP | non-coding | IVS14 + 58 | | G/C | NA | NA | 0.003 | 0 |
| FPGS_9196 | 14 | SNP | non-coding | IVS14 + 59 | | G/T | NA | NA | 0.003 | 0 |
| FPGS_11475 | 15 | SNP | Synonymous | A503A | | G/A | NA | NA | 0.005 | 0.999 |

TABLE N
GART Genetic Variants

| | | | | | | | Poly | | | |
|---|---|---|---|---|---|---|---|---|---|---|
| GENE_position | Exon | Type | Function | Location | dB SNP id | Change | Phen | SIFT | MAF | HWE |
| GART_3782 | 2 | SNP | non- | 5′UTR | | G/T | NA | NA | 0.001 | 1 |
| | | | coding | | | | | | | |
| GART_3842 | 2 | SNP | Nonsynonymous | T16M | | C/T | Probably | Gene too | 0.001 | 1 |
| | | | | | | | Damaging | big for | | |
| | | | | | | | | SIFT | | |
| GART_7745 | 3 | SNP | non- | IVS2 − 46 | | G/T | NA | NA | 0.001 | 1 |
| | | | coding | | | | | | | |
| GART_7984 | 3 | SNP | non- | IVS3 + 98 | | C/T | NA | NA | 0.001 | 1 |
| | | | coding | | | | | | | |
| GART_10720 | 5 | SNP | Nonsynonymous | A161G | rs35035222 | C/G | Possibly | Gene too | 0.001 | 1 |
| | | | | | | | Damaging | big for | | |
| | | | | | | | | SIFT | | |
| GART_10775 | 5 | SNP | non- | IVS5 + 9 | | A/G | NA | NA | 0.001 | 1 |
| | | | coding | | | | | | | |
| GART_11521 | 6 | SNP | non- | IVS5 − 33 | | A/T | NA | NA | 0.009 | |
| | | | coding | | | | | | | |
| GART_11522 | 6 | SNP | non- | IVS5 − 32 | | A/T | NA | NA | 0.001 | 1 |
| | | | coding | | | | | | | |
| GART_11541 | 6 | SNP | non- | IVS5 − 13 | | A/C | NA | NA | 0.002 | |
| | | | coding | | | | | | | |
| GART_12356 | 7 | SNP | non- | IVS7 + 4 | | C/T | NA | NA | 0.001 | 1 |
| | | | coding | | | | | | | |
| GART_14200 | 8 | SNP | Synonymous | I250I | | C/T | NA | NA | 0.001 | 1 |
| GART_14273 | 8 | SNP | non- | IVS8 + 12 | | C/T | NA | NA | 0.001 | 1 |
| | | | coding | | | | | | | |
| GART_14282 | 8 | SNP | non- | IVS8 + 21 | | A/G | NA | NA | 0.001 | 1 |
| | | | coding | | | | | | | |
| GART_14739 | 10 | SNP | non- | IVS9 − 37 | | A/C | NA | NA | 0.001 | 1 |
| | | | coding | | | | | | | |
| GART_14781 | 10 | SNP | Synonymous | I301I | | C/T | NA | NA | 0.001 | 1 |
| GART_18055 | 11 | SNP | non- | IVS10 − | | C/T | NA | NA | 0.001 | 1 |
| | | | coding | 55 | | | | | | |
| GART_18064 | 11 | SNP | non- | IVS10 − | | A/G | NA | NA | 0.001 | 1 |
| | | | coding | 46 | | | | | | |
| GART_18130 | 11 | SNP | Nonsynonymous | L363I | | A/C | Benign | Gene too | 0.001 | 1 |
| | | | | | | | | big for | | |
| | | | | | | | | SIFT | | |
| GART_18142 | 11 | SNP | Nonsynonymous | V367M | | A/G | Possibly | Gene too | 0.001 | 1 |
| | | | | | | | Damaging | big for | | |
| | | | | | | | | SIFT | | |
| GART_18197 | 11 | SNP | Nonsynonymous | R385K | | A/G | Probably | Gene too | 0.001 | 1 |
| | | | | | | | Damaging | big for | | |
| | | | | | | | | SIFT | | |
| GART_18232 | 11 | SNP | Nonsynonymous | I397V | | A/G | Benign | Gene too | 0.001 | 1 |
| | | | | | | | | big for | | |
| | | | | | | | | SIFT | | |
| GART_18304 | 11 | SNP | Nonsynonymous | V421I | rs8788 | A/G | Possibly | Gene too | 0.137 | 0.046 |
| | | | | | | | Damaging | big for | | |
| | | | | | | | | SIFT | | |
| GART_18401 | 11 | SNP | non- | IVS11 + 60 | | A/T | NA | NA | 0.002 | 0.967 |
| | | | coding | | | | | | | |
| GART_20794 | 12 | SNP | non- | IVS11 − | rs2834234 | A/G | NA | NA | 0.136 | 0.079 |
| | | | coding | 34 | | | | | | |
| GART_20812 | 12 | SNP | non- | IVS11 − | | A/G | NA | NA | 0.048 | 1 |
| | | | coding | 16 | | | | | | |
| GART_20825 | 12 | SNP | non- | IVS11 − 3 | | C/T | NA | NA | 0.002 | 0.999 |
| | | | coding | | | | | | | |
| GART_20862 | 12 | SNP | Nonsynonymous | A445T | | A/G | Benign | Gene too | 0.001 | 1 |
| | | | | | | | | big for | | |
| | | | | | | | | SIFT | | |
| GART_22073 | 13 | SNP | non- | IVS12 − | rs2834233 | C/T | NA | NA | 0.173 | 0.15 |
| | | | coding | 22 | | | | | | |
| GART_22481 | 14 | SNP | non- | IVS13 − | | A/G | NA | NA | 0.003 | 0.998 |
| | | | coding | 67 | | | | | | |
| GART_22521 | 14 | SNP | non- | IVS13 − | rs2834232 | A/G | NA | NA | 0.2 | 0.264 |
| | | | coding | 27 | | | | | | |
| GART_22573 | 14 | SNP | Nonsynonymous | D510G | rs35927582 | A/G | Benign | Gene too | 0.001 | 1 |
| | | | | | | | | big for | | |
| | | | | | | | | SIFT | | |
| GART_25425 | 15 | SNP | non- | IVS14 − | | A/G | NA | NA | 0.001 | 1 |
| | | | coding | 77 | | | | | | |
| GART_25433 | 15 | SNP | non- | IVS14 − | | C/G | NA | NA | 0.015 | 0.946 |
| | | | coding | 69 | | | | | | |
| GART_25601 | 15 | SNP | Nonsynonymous | H601R | | A/G | Benign | Gene too | 0.001 | 1 |
| | | | | | | | | big for | | |
| | | | | | | | | SIFT | | |
| GART_25694 | 15 | SNP | Nonsynonymous | A632V | rs59920090 | C/T | Benign | Gene too | 0.011 | 0.972 |
| | | | | | | | | big for | | |
| | | | | | | | | SIFT | | |
| GART_25720 | 15 | SNP | Nonsynonymous | P641A | rs34588874 | C/G | Benign | Gene too | 0.002 | 0.999 |
| | | | | | | | | big for | | |
| | | | | | | | | SIFT | | |
| GART_25867 | 16 | SNP | non- | IVS15 − | | C/T | NA | NA | 0.001 | 1 |
| | | | coding | 102 | | | | | | |
| GART_25912 | 16 | SNP | non- | IVS15 − | | C/T | NA | NA | 0.101 | 0.989 |
| | | | coding | 57 | | | | | | |
| GART_25951 | 16 | SNP | non- | IVS15 − | | C/T | NA | NA | 0.001 | 1 |
| | | | coding | 18 | | | | | | |
| GART_25956 | 16 | indel | non- | IVS15 − | | delCT | NA | NA | 0.001 | 1 |
| | | | coding | 13 | | | | | | |
| GART_26127 | 16 | SNP | non- | IVS16 + 6 | | A/G | NA | NA | 0.001 | 1 |
| | | | coding | | | | | | | |
| GART_26195 | 16 | SNP | non- | IVS16 + 74 | | C/G | NA | NA | 0.001 | 1 |
| | | | coding | | | | | | | |
| GART_31619 | 17 | SNP | non- | IVS16 − | rs7281488 | A/G | NA | NA | 0.009 | 0.983 |
| | | | coding | 33 | | | | | | |
| GART_31627 | 17 | SNP | non- | IVS16 − | | A/T | NA | NA | 0.001 | 1 |
| | | | coding | 25 | | | | | | |
| GART_31641 | 17 | SNP | non- | IVS16 − | | A/G | NA | NA | 0.001 | 1 |
| | | | coding | 11 | | | | | | |
| GART_31799 | 17 | SNP | Nonsynonymous | D752G | rs8971 | A/G | Benign | Gene too | 0.202 | 0.431 |
| | | | | | | | | big for | | |
| | | | | | | | | SIFT | | |
| GART_31887 | 17 | SNP | non- | IVS17 + 29 | | C/T | NA | NA | 0.001 | 1 |
| | | | coding | | | | | | | |
| GART_31902 | 17 | SNP | non- | IVS17 + 44 | | A/G | NA | NA | 0.001 | 1 |
| | | | coding | | | | | | | |
| GART_31933 | 17 | SNP | non- | IVS17 + 75 | | A/C | NA | NA | 0.001 | 1 |
| | | | coding | | | | | | | |
| GART_33173 | 18 | SNP | non- | IVS17 − | | A/G | NA | NA | 0.001 | 1 |
| | | | coding | 17 | | | | | | |
| GART_33264 | 18 | SNP | Nonsynonymous | L797M | | A/C | Benign | Gene too | 0.001 | 1 |
| | | | | | | | | big for | | |
| | | | | | | | | SIFT | | |
| GART_33286 | 18 | SNP | Nonsynonymous | E804A | | A/C | Benign | Gene too | 0.005 | 0.995 |
| | | | | | | | | big for | | |
| | | | | | | | | SIFT | | |
| GART_36963 | 19 | SNP | non- | IVS18 − | | A/G | NA | NA | 0.001 | 1 |
| | | | coding | 43 | | | | | | |
| GART_36964 | 19 | SNP | non- | IVS18 − | | A/T | NA | NA | 0.009 | 0.983 |
| | | | coding | 42 | | | | | | |
| GART_36967 | 19 | SNP | non- | IVS18 − | rs2070390 | A/T | NA | NA | 0.204 | 0.622 |
| | | | coding | 39 | | | | | | |
| GART_37428 | 20 | SNP | Synonymous | Y868Y | | C/T | NA | NA | 0.003 | 0.99 |
| GART_37433 | 20 | SNP | Nonsynonymous | N870S | | A/G | Benign | Gene too | 0.001 | 1 |
| | | | | | | | | big for | | |
| | | | | | | | | SIFT | | |
| GART_38709 | 21 | SNP | non- | IVS21 + 11 | rs2070388 | C/G | NA | NA | 0.213 | 0.985 |
| | | | coding | | | | | | | |
| GART_38762 | 22 | SNP | non- | IVS21 − | | A/G | NA | NA | 0.001 | 1 |
| | | | coding | 33 | | | | | | |
| GART_38914 | 22 | SNP | Synonymous | A987A | | A/C | NA | NA | 0.001 | 1 |
| GART_38989 | 22 | SNP | non- | 3′ UTR | | C/G | NA | NA | 0.001 | 1 |
| | | | coding | | | | | | | |

TABLE O
MAT1A Genetic Variants

| | | | | | | | Poly | | | |
|---|---|---|---|---|---|---|---|---|---|---|
| GENE_position | Exon | Type | Function | Location | dB SNP id | Change | Phen | SIFT | MAF | HWE |
| MAT1A_5045 | 2 | SNP | non- | IVS1 − 45 | | A/T | NA | NA | 0.001 | 1 |
| | | | coding | | | | | | | |
| MAT1A_5081 | 2 | SNP | non- | IVS1 − 9 | rs10887721 | C/G | NA | NA | 0.133 | 0.083 |
| | | | coding | | | | | | | |
| MAT1A_5181 | 2 | SNP | non- | IVS2 + 14 | | A/G | NA | NA | 0.008 | 0.987 |
| | | | coding | | | | | | | |
| MAT1A_5233 | 2 | SNP | non- | IVS2 + 66 | | A/G | NA | NA | 0.002 | 0.999 |
| | | | coding | | | | | | | |
| MAT1A_6739 | 3 | SNP | Nonsynonymous | I90V | | A/G | Benign | Not | 0.001 | 1 |
| | | | | | | | | available | | |
| MAT1A_6795 | 3 | SNP | non- | IVS3 + 32 | | G/T | NA | NA | 0.001 | 1 |
| | | | coding | | | | | | | |
| MAT1A_9833 | 4 | SNP | non- | IVS3 − 54 | | C/T | NA | NA | 0.049 | 0.993 |
| | | | coding | | | | | | | |
| MAT1A_10006 | 4 | SNP | non- | IVS4 + 7 | | C/T | NA | NA | 0.003 | 0.998 |
| | | | coding | | | | | | | |
| MAT1A_10089 | 4 | SNP | non- | IVS4 + 90 | rs2282367 | C/T | NA | NA | 0.201 | 0.122 |
| | | | coding | | | | | | | |
| MAT1A_10312 | 5 | SNP | non- | IVS4 − 51 | | C/T | NA | NA | 0.041 | 0.337 |
| | | | coding | | | | | | | |
| MAT1A_10339 | 5 | SNP | non- | IVS4 − 24 | | A/G | NA | NA | 0.002 | 0.999 |
| | | | coding | | | | | | | |
| MAT1A_10374 | 5 | SNP | Synonymous | F139F | | C/T | NA | NA | 0.001 | 1 |
| MAT1A_10383 | 5 | SNP | Synonymous | A142A | rs1143694 | C/T | NA | NA | 0.204 | 0.001 |
| MAT1A_10484 | 5 | SNP | Nonsynonymous | L176R | | G/T | Probably | Not | 0.001 | 1 |
| | | | | | | | Damaging | available | | |
| MAT1A_10555 | 5 | SNP | non- | IVS5 + 49 | | A/C | NA | NA | 0.001 | 1 |
| | | | coding | | | | | | | |
| MAT1A_14038 | 6 | SNP | non- | IVS5 − 47 | | A/G | NA | NA | 0.04 | 0.34 |
| | | | coding | | | | | | | |
| MAT1A_14114 | 6 | SNP | Synonymous | G193G | | C/T | NA | NA | 0.001 | 1 |
| MAT1A_14177 | 6 | SNP | Synonymous | T214T | | A/G | NA | NA | 0.001 | 1 |
| MAT1A_15424 | 7 | SNP | non- | IVS6 − 56 | | A/C | NA | NA | 0.001 | 1 |
| | | | coding | | | | | | | |
| MAT1A_15500 | 7 | SNP | Synonymous | G263G | | C/T | NA | NA | 0.002 | 0.999 |
| MAT1A_15581 | 7 | SNP | Synonymous | V290V | rs10788546 | A/G | NA | NA | 0.221 | 0.579 |
| MAT1A_15593 | 7 | SNP | Synonymous | A294A | rs10887711 | C/T | NA | NA | 0.221 | 0.579 |
| MAT1A_15596 | 7 | SNP | Synonymous | A295A | rs17851642 | A/T | NA | NA | 0.001 | 1 |
| MAT1A_15646 | 7 | SNP | Nonsynonymous | R312Q | | A/G | Benign | Not | 0.001 | 1 |
| | | | | | | | | available | | |
| MAT1A_15706 | 7 | SNP | non- | IVS7 + 44 | | C/T | NA | NA | 0.186 | 0.068 |
| | | | coding | | | | | | | |
| MAT1A_15715 | 7 | SNP | non- | IVS7 + 53 | | AG | NA | NA | 0.001 | 1 |
| | | | coding | | | | | | | |
| MAT1A_15730 | 7 | indel | non- | IVS7 + 68 | | delA | NA | NA | 0.001 | 1 |
| | | | coding | | | | | | | |
| MAT1A_15758 | 7 | SNP | non- | IVS7 + 96 | | C/T | NA | NA | 0.016 | 0.94 |
| | | | coding | | | | | | | |
| MAT1A_15760 | 7 | SNP | non- | IVS7 + 98 | rs10788545 | C/T | NA | NA | 0.202 | 0.27 |
| | | | coding | | | | | | | |
| MAT1A_16133 | 8 | SNP | Synonymous | F353F | | C/T | NA | NA | 0.001 | 1 |
| MAT1A_16173 | 8 | SNP | non- | IVS8 + 14 | rs2994388 | C/T | NA | NA | 0.462 | 0.993 |
| | | | coding | | | | | | | |
| MAT1A_16174 | 8 | SNP | non- | IVS8 + 15 | | A/G | NA | NA | 0.002 | 0.999 |
| | | | coding | | | | | | | |
| MAT1A_16218 | 8 | SNP | non- | IVS8 + 59 | | A/T | NA | NA | 0.001 | 1 |
| | | | coding | | | | | | | |
| MAT1A_16752 | 9 | SNP | non- | IVS8 − 44 | rs4933327 | C/T | NA | NA | 0.229 | 0.608 |
| | | | coding | | | | | | | |
| MAT1A_16841 | 9 | SNP | Synonymous | Y377Y | rs2993763 | C/T | NA | NA | 0.46 | 0.996 |
| MAT1A_16965 | 9 | SNP | non- | 3′ UTR | rs7087728 | C/T | NA | NA | 0.245 | 0.628 |
| | | | coding | | | | | | | |
| MAT1A_16971 | 9 | SNP | non- | 3′ UTR | | G/T | NA | NA | 0.002 | 0.99 |
| | | | coding | | | | | | | |

TABLE P
MAT2A Genetic Variants

| | | | | | | | Poly | | | |
|---|---|---|---|---|---|---|---|---|---|---|
| GENE_position | Exon | Type | Function | Location | dB SNP id | Change | Phen | SIFT | MAF | HWE |
| MAT2A_2871 | 2 | SNP | non-coding | IVS1 − 48 | | A/C | NA | NA | 0.001 | 1 |
| MAT2A_2873 | 2 | indel | non-coding | IVS1 − 50 | | insATAC | NA | NA | 0.009 | 0.982 |
| MAT2A_2939 | 2 | SNP | Synonymous | Q36Q | | A/G | NA | NA | 0.001 | 1 |
| MAT2A_3047 | 3 | SNP | non-coding | IVS2 − 48 | rs58507836 | A/G | NA | NA | 0.005 | 0.993 |
| MAT2A_3287 | 3 | SNP | non-coding | IVS3 + 70 | | A/G | NA | NA | 0.012 | 0.966 |
| MAT2A_3394 | 4 | SNP | non-coding | IVS3 − 79 | | C/T | NA | NA | 0.006 | 0.99 |
| MAT2A_3466 | 4 | SNP | non-coding | IVS3 − 7 | | C/G | NA | NA | 0.001 | 1 |
| MAT2A_3498 | 4 | SNP | Synonymous | V106V | rs72940560 | G/T | NA | NA | 0.002 | 0.999 |
| MAT2A_3617 | 4 | SNP | non-coding | IVS4 + 32 | rs62620249 | C/T | NA | NA | 0.008 | 0.983 |
| MAT2A_3650 | 5 | SNP | non-coding | IVS4 − 19 | | A/G | NA | NA | 0.003 | 0.998 |
| MAT2A_3704 | 5 | SNP | Synonymous | E147E | | A/G | NA | NA | 0.001 | 1 |
| MAT2A_3963 | 6 | SNP | non-coding | IVS5 − 32 | rs1078005 | A/G | NA | NA | 0.005 | 0.993 |
| MAT2A_4174 | 6 | SNP | Synonymous | H243H | | C/T | NA | NA | 0.001 | 1 |
| MAT2A_4428 | 7 | SNP | Synonymous | R264R | rs1078004 | C/G | NA | NA | 0.4 | 0.65 |
| MAT2A_4449 | 7 | SNP | Synonymous | Y271Y | | C/T | NA | NA | 0.001 | 1 |
| MAT2A_4476 | 7 | SNP | Synonymous | G280G | | C/T | NA | NA | 0.001 | 1 |
| MAT2A_4608 | 7 | SNP | non-coding | IVS7 + 21 | | C/G | NA | NA | 0.001 | 1 |
| MAT2A_4660 | 8 | SNP | non-coding | IVS7 − 81 | | C/G | NA | NA | 0.001 | 1 |
| MAT2A_4692 | 8 | SNP | non-coding | IVS7 − 49 | | A/G | NA | NA | 0.228 | 0.151 |
| MAT2A_4931 | 8 | indel | non-coding | IVS8 + 53 | | insGT | NA | NA | 0.003 | 0.997 |
| MAT2A_5313 | 9 | SNP | non-coding | IVS8 − 199 | | C/T | NA | NA | 0.001 | 1 |
| MAT2A_5460 | 9 | indel | non-coding | IVS8 − 54 | | insT | NA | NA | 0.001 | 1 |
| MAT2A_5480 | 9 | SNP | non-coding | IVS8 − 33 | | C/T | NA | NA | 0.001 | 1 |

TABLE Q
MTHFD1 Genetic Variants

| | | | | | dB SNP | | | | | |
|---|---|---|---|---|---|---|---|---|---|---|
| GENE_position | Exon | Type | Function | Location | id | Change | PolyPhen | SIFT | MAF | HWE |
| MTHFD1_1039 | 1 | SNP | Non- | 5′UTR | | G/A | NA | NA | 0.001 | 1 |
| | | | coding | | | | | | | |
| MTHFD1_1176 | 1 | indel | Non- | IVS1 + 81 | | insT | NA | NA | 0.001 | 1 |
| | | | coding | | | | | | | |
| MTHFD1_1181 | 1 | indel | Non- | IVS1 + 86 | | delG | NA | NA | 0.001 | 1 |
| | | | coding | | | | | | | |
| MTHFD1_13431 | 2 | SNP | Nonsynonymous | A18V | | C/T | Benign | Tolerated | 0.004 | 0.996 |
| MTHFD1_13645 | 2 | SNP | Non- | IVS2 + 141 | | C/G | NA | NA | 0.001 | 1 |
| | | | coding | | | | | | | |
| MTHFD1_13709 | 2 | SNP | Non- | IVS2 + 205 | | T/C | NA | NA | 0.005 | 0.993 |
| | | | coding | | | | | | | |
| MTHFD1_23841 | 3 | indel | Non- | IVS3 + 64 | | insC | NA | NA | 0.001 | 1 |
| | | | coding | | | | | | | |
| MTHFD1_23859 | 3 | SNP | Non- | IVS3 + 82 | | C/T | NA | NA | 0.001 | 1 |
| | | | coding | | | | | | | |
| MTHFD1_28290 | 6 | SNP | Nonsynonymous | K134R | rs1950902 | G/A | Benign | Tolerated | 0.149 | 0.665 |
| MTHFD1_28357 | 6 | SNP | Synonymous | I156I | | C/T | NA | NA | 0.001 | 1 |
| MTHFD1_28378 | 6 | SNP | Non- | IVS6 + 11 | | C/T | NA | NA | 0.001 | 1 |
| | | | coding | | | | | | | |
| MTHFD1_30523 | 7 | SNP | Synonymous | P162P | | G/A | NA | NA | 0.001 | 1 |
| MTHFD1_30529 | 7 | SNP | Synonymous | A164A | | C/T | NA | NA | 0.001 | 1 |
| MTHFD1_30656 | 7 | SNP | Non- | IVS7 + 4 | | G/C | NA | NA | 0.001 | 1 |
| | | | coding | | | | | | | |
| MTHFD1_30729 | 7 | SNP | Non- | IVS7 + 77 | | C/T | NA | NA | 0.001 | 1 |
| | | | coding | | | | | | | |
| MTHFD1_32375 | 8 | SNP | Non- | IVS7 − 67 | | T/C | NA | NA | 0.002 | 0.999 |
| | | | coding | | | | | | | |
| MTHFD1_32564 | 8 | SNP | Non- | IVS8 + 11 | | T/C | NA | NA | 0.002 | 0.999 |
| | | | coding | | | | | | | |
| MTHFD1_37632 | 9 | indel | Non- | IVS9 + 73 | | delAG | NA | NA | 0.003 | 0.998 |
| | | | coding | | | AAAT | | | | |
| | | | | | | GT | | | | |
| MTHFD1_37663 | 9 | SNP | Non- | IVS9 + 104 | rs61290360 | A/G | NA | NA | 0.033 | 0.776 |
| | | | coding | | | | | | | |
| MTHFD1_37713 | 9 | SNP | Non- | IVS9 + 154 | | G/A | NA | NA | 0.001 | 1 |
| | | | coding | | | | | | | |
| MTHFD1_38549 | 11 | SNP | Non- | IVS10 − | | C/A | NA | NA | 0.021 | 0.995 |
| | | | coding | 98 | | | | | | |
| MTHFD1_38676 | 11 | SNP | Nonsynonymous | P328L | | C/T | Benign | Tolerated | 0.005 | 1 |
| MTHFD1_42693 | 13 | SNP | Non- | IVS12 − | rs61107070 | C/G | NA | NA | 0.042 | 0 |
| | | | coding | 119 | | | | | | |
| MTHFD1_42866 | 13 | SNP | Non- | IVS13 + 8 | | A/G | NA | NA | 0.071 | 0 |
| | | | coding | | | | | | | |
| MTHFD1_42907 | 13 | SNP | Non- | IVS13 + 49 | | G/T | NA | NA | 0.001 | 1 |
| | | | coding | | | | | | | |
| MTHFD1_42912 | 13 | indel | Non- | IVS13 + 54 | rs60870392 | delG | NA | NA | 0.039 | 0 |
| | | | coding | | | | | | | |
| MTHFD1_42913 | 13 | indel | Non- | IVS13 + 54 | | insG | NA | NA | 0.003 | 0 |
| | | | coding | | | | | | | |
| MTHFD1_42929 | 13 | SNP | Non- | IVS13 + 70 | rs59096477 | C/A | NA | NA | 0.041 | 0 |
| | | | coding | | | | | | | |
| MTHFD1_42979 | 13 | SNP | Non- | IVS13 + 120 | | G/C | NA | NA | 0.001 | 1 |
| | | | coding | | | | | | | |
| MTHFD1_43104 | 13 | indel | Non- | IVS13 + 208 | | insACA | NA | NA | 0.001 | 1 |
| | | | coding | | | GGCA | | | | |
| | | | | | | TGCA | | | | |
| | | | | | | CCAC | | | | |
| | | | | | | CACG | | | | |
| | | | | | | CTCA | | | | |
| | | | | | | GCTA | | | | |
| | | | | | | ATTTT | | | | |
| | | | | | | GTATT | | | | |
| MTHFD1_43174 | 13 | indel | Non- | IVS13 + 278 | | delA | NA | NA | 0.001 | 1 |
| | | | coding | | | | | | | |
| MTHFD1_43237 | 13 | SNP | Non- | IVS13 + 341 | | T/C | NA | NA | 0.001 | 1 |
| | | | coding | | | | | | | |
| MTHFD1_44133 | 14 | SNP | Non- | IVS13 − | | A/G | NA | NA | 0.001 | 1 |
| | | | coding | 65 | | | | | | |
| MTHFD1_44407 | 15 | SNP | Non- | IVS14 − | rs60806768 | A/G | NA | NA | 0.035 | 0.15 |
| | | | coding | 46 | | | | | | |
| MTHFD1_44411 | 15 | SNP | Non- | IVS14 − | rs59770063 | G/A | NA | NA | 0.035 | 0.182 |
| | | | coding | 42 | | | | | | |
| MTHFD1_44419 | 15 | SNP | Non- | IVS14 − | | C/G | NA | NA | 0.001 | 1 |
| | | | coding | 34 | | | | | | |
| MTHFD1_44540 | 15 | indel | Non- | IVS15 + 13 | | delT | NA | NA | 0.002 | 0.999 |
| | | | coding | | | | | | | |
| MTHFD1_44594 | 15 | SNP | Non- | IVS15 + 67 | | A/C | NA | NA | 0.004 | 0.996 |
| | | | coding | | | | | | | |
| MTHFD1_48301 | 16 | SNP | Synonymous | L521L | | T/C | NA | NA | 0.001 | 1 |
| MTHFD1_48389 | 16 | SNP | Non- | IVS16 + 52 | | A/T | NA | NA | 0.001 | 1 |
| | | | coding | | | | | | | |
| MTHFD1_48420 | 16 | SNP | Non- | IVS16 + 83 | | G/A | NA | NA | 0.001 | 1 |
| | | | coding | | | | | | | |
| MTHFD1_48441 | 16 | SNP | Non- | IVS16 + 104 | rs3818240 | T/C | NA | NA | 0.168 | 0.692 |
| | | | coding | | | | | | | |
| MTHFD1_51924 | 17 | SNP | Non- | IVS17 + 86 | rs45618332 | C/T | NA | NA | 0.181 | 0.857 |
| | | | coding | | | | | | | |
| MTHFD1_51954 | 17 | SNP | Non- | IVS17 + 116 | | C/G | NA | NA | 0.001 | 1 |
| | | | coding | | | | | | | |
| MTHFD1_52960 | 18 | SNP | Non- | IVS18 + 28 | | C/T | NA | NA | 0.001 | 1 |
| | | | coding | | | | | | | |
| MTHFD1_54149 | 19 | SNP | Non- | IVS19 + 30 | | T/C | NA | NA | 0.002 | 0.999 |
| | | | coding | | | | | | | |
| MTHFD1_54173 | 19 | SNP | Non- | IVS19 + 54 | | G/G | NA | NA | 0.001 | 1 |
| | | | coding | | | | | | | |
| MTHFD1_54205 | 19 | SNP | Non- | IVS19 + 86 | | G/A | NA | NA | 0.001 | 1 |
| | | | coding | | | | | | | |
| MTHFD1_54206 | 19 | SNP | Non- | IVS19 + 87 | | C/T | NA | NA | 0.002 | 0.999 |
| | | | coding | | | | | | | |
| MTHFD1_54240 | 19 | SNP | Non- | IVS19 + 121 | | A/G | NA | NA | 0.001 | 1 |
| | | | coding | | | | | | | |
| MTHFD1_54243 | 19 | SNP | Non- | IVS19 + 124 | | T/C | NA | NA | 0.002 | 0.999 |
| | | | coding | | | | | | | |
| MTHFD1_54247 | 19 | SNP | Non- | IVS19 + 128 | rs35519051 | C/T | NA | NA | 0.022 | 0.897 |
| | | | coding | | | | | | | |
| MTHFD1_54248 | 19 | SNP | Non- | IVS19 + 129 | rs35519051 | A/G | NA | NA | 0.022 | 0.887 |
| | | | coding | | | | | | | |
| MTHFD1_54270 | 19 | SNP | Non- | IVS19 + 151 | | A/G | NA | NA | 0.001 | 1 |
| | | | coding | | | | | | | |
| MTHFD1_54276 | 19 | SNP | Non- | IVS19 + 157 | | T/G | NA | NA | 0.008 | 0.986 |
| | | | coding | | | | | | | |
| MTHFD1_54278 | 19 | SNP | Non- | IVS19 + 159 | | C/G | NA | NA | 0.008 | 0.986 |
| | | | coding | | | | | | | |
| MTHFD1_54283 | 19 | SNP | Non- | IVS19 + 164 | | T/C | NA | NA | 0.008 | 0.986 |
| | | | coding | | | | | | | |
| MTHFD1_54288 | 19 | SNP | Non- | IVS19 + 169 | rs7147830 | T/C | NA | NA | 0.002 | 0.999 |
| | | | coding | | | | | | | |
| MTHFD1_54314 | 19 | indel | Non- | IVS19 + 194 | | insA | NA | NA | 0.001 | 1 |
| | | | coding | | | | | | | |
| MTHFD1_54334 | 19 | SNP | Non- | IVS19 + 214 | | A/G | NA | NA | 0.001 | 1 |
| | | | coding | | | | | | | |
| MTHFD1_54346 | 19 | SNP | Non- | IVS19 + 226 | | G/A | NA | NA | 0.004 | 0.996 |
| | | | coding | | | | | | | |
| MTHFD1_54426 | 20 | SNP | Non- | IVS19 − | | C/T | NA | NA | 0.005 | 0.993 |
| | | | coding | 295 | | | | | | |
| MTHFD1_54449 | 20 | SNP | Non- | IVS19 − | | G/A | NA | NA | 0.003 | 0.998 |
| | | | coding | 272 | | | | | | |
| MTHFD1_54457 | 20 | SNP | Non- | IVS19 − | | G/A | NA | NA | 0.019 | 0.921 |
| | | | coding | 264 | | | | | | |
| MTHFD1_54461 | 20 | SNP | Non- | IVS19 − | | C/G | NA | NA | 0.001 | 1 |
| | | | coding | 260 | | | | | | |
| MTHFD1_54701 | 20 | SNP | Non- | IVS19 − | | C/T | NA | NA | 0.001 | 1 |
| | | | coding | 20 | | | | | | |
| MTHFD1_54794 | 20 | SNP | Nonsynonymous | R653Q | rs2236225 | A/G | Benign | Tolerated | 0.5 | 0.712 |
| MTHFD1_54891 | 21 | SNP | Non- | IVS20 − | | A/G | NA | NA | 0.001 | 1 |
| | | | coding | 39 | | | | | | |
| MTHFD1_55100 | 21 | SNP | Non- | IVS21 + 31 | rs2236224 | G/A | NA | NA | 0.48 | 0.813 |
| | | | coding | | | | | | | |
| MTHFD1_61041 | 23 | SNP | Non- | IVS23 + 57 | | G/A | NA | NA | 0.001 | 1 |
| | | | coding | | | | | | | |
| MTHFD1_62114 | 23 | SNP | Nonsynonymous | T761M | rs10813 | C/T | Probably | Affected | 0.01 | 0.978 |
| | | | | | | | Damaging | | | |
| MTHFD1_62130 | 23 | SNP | Nonsynonymous | E766D | | G/C | Possibly | Affected | 0.001 | 1 |
| | | | | | | | Damaging | | | |
| MTHFD1_62137 | 23 | SNP | Nonsynonymous | L769F | rs17857382 | C/T | Possibly | Tolerated | 0.01 | 0.973 |
| | | | | | | | Damaging | | | |
| MTHFD1_62146 | 23 | SNP | Nonsynonymous | R772C | | C/T | Possibly | Affected | 0.001 | 1 |
| | | | | | | | Damaging | | | |
| MTHFD1_62147 | 23 | SNP | Nonsynonymous | R772H | | G/A | Benign | Tolerated | 0.003 | 0.998 |
| MTHFD1_62327 | 24 | SNP | Non- | IVS24 + 38 | | G/A | NA | NA | 0.001 | 1 |
| | | | coding | | | | | | | |
| MTHFD1_62355 | 24 | SNP | Non- | IVS24 + 66 | | G/T | NA | NA | 0.001 | 1 |
| | | | coding | | | | | | | |
| MTHFD1_62375 | 24 | SNP | Non- | IVS24 + 86 | rs10138064 | A/T | NA | NA | 0.001 | 1 |
| | | | coding | | | | | | | |
| MTHFD1_62396 | 24 | SNP | Non- | IVS24 + 107 | | G/A | NA | NA | 0.001 | 1 |
| | | | coding | | | | | | | |
| MTHFD1_62397 | 24 | SNP | Non- | IVS24 + 108 | | C/A | NA | NA | 0.001 | 1 |
| | | | coding | | | | | | | |
| MTHFD1_66378 | 25 | indel | Non- | IVS24 − | | delAT | NA | NA | 0.001 | 1 |
| | | | coding | 43 | | | | | | |
| MTHFD1_66614 | 25 | SNP | Non- | IVS25 + 86 | rs1256146 | G/A | NA | NA | 0.159 | 0.993 |
| | | | coding | | | | | | | |
| MTHFD1_67368 | 26 | SNP | Non- | IVS25 − | | C/T | NA | NA | 0.004 | 0.996 |
| | | | coding | 22 | | | | | | |
| MTHFD1_67575 | 26 | SNP | Non- | IVS26 + 33 | | A/G | NA | NA | 0.004 | 0.996 |
| | | | coding | | | | | | | |
| MTHFD1_70629 | 27 | SNP | Non- | IVS26 − | | C/T | NA | NA | 0.001 | 1 |
| | | | coding | 252 | | | | | | |
| MTHFD1_70756 | 27 | indel | Non- | IVS26 − | rs57610847 | delC | NA | NA | 0.001 | 1 |
| | | | coding | 125 | | | | | | |
| MTHFD1_70807 | 27 | SNP | Non- | IVS26 − | | C/T | NA | NA | 0.001 | 1 |
| | | | coding | 74 | | | | | | |
| MTHFD1_71034 | 27 | SNP | Non- | IVS27 + 60 | | T/C | NA | NA | 0.003 | 0.998 |
| | | | coding | | | | | | | |

TABLE R
MTHFD2 Genetic Variants

| GENE_position | Exon | Type | Function | Location | dB SNP id | Change | PolyPhen | SIFT | MAF | HWE |
|---|---|---|---|---|---|---|---|---|---|---|
| MTHFD2_1035 | 1 | SNP | non-coding | 5′UTR | | G/C | NA | NA | 0.001 | 1 |
| MTHFD2_1044 | 1 | SNP | non-coding | 5′UTR | | C/T | NA | NA | 0.001 | 1 |
| MTHFD2_1052 | 1 | SNP | non-coding | 5′UTR | | C/T | NA | NA | 0.001 | 1 |
| MTHFD2_1060 | 1 | SNP | non-coding | 5′UTR | | C/G | NA | NA | 0.001 | 1 |
| MTHFD2_1243 | 1 | SNP | non-coding | IVS1 + 63 | rs3821321 | G/A | NA | NA | 0.286 | 0.019 |
| MTHFD2_1258 | 1 | SNP | non-coding | IVS1 + 78 | rs13001449 | G/T | NA | NA | 0.04 | 0.305 |
| MTHFD2_8108 | 2 | SNP | non-coding | IVS1 − 35 | | G/C | NA | NA | 0.063 | 0.994 |
| MTHFD2_10123 | 3 | SNP | non-coding | IVS2 − 19 | | T/C | NA | NA | 0.001 | 1 |
| MTHFD2_10417 | 3 | SNP | non-coding | IVS3 + 153 | | G/C | NA | NA | 0.002 | 0.999 |
| MTHFD2_10469 | 3 | SNP | non-coding | IVS3 + 205 | rs10209904 | C/G | NA | NA | 0.009 | 0.983 |
| MTHFD2_10926 | 4 | SNP | non-coding | IVS3 − 81 | | T/G | NA | NA | 0.003 | 0.998 |
| MTHFD2_10929 | 4 | SNP | non-coding | IVS3 − 78 | | C/T | NA | NA | 0.001 | 1 |
| MTHFD2_10930 | 4 | SNP | non-coding | IVS3 − 77 | rs9282785 | G/A | NA | NA | 0.001 | 1 |
| MTHFD2_10937 | 4 | SNP | non-coding | IVS3 − 70 | rs2293342 | A/G | NA | NA | 0.006 | 0.99 |
| MTHFD2_11083 | 4 | SNP | Synonymous | V162V | | A/G | NA | NA | 0.001 | 1 |
| MTHFD2_12359 | 5 | SNP | non-coding | IVS4 − 21 | | T/C | NA | NA | 0.001 | 1 |
| MTHFD2_13617 | 6 | SNP | non-coding | IVS5 − 20 | | A/G | NA | NA | 0.001 | 1 |
| MTHFD2_13627 | 6 | SNP | non-coding | IVS5 − 10 | | T/C | NA | NA | 0.004 | 0.996 |
| MTHFD2_14024 | 7 | SNP | non-coding | IVS6 − 155 | | A/G | NA | NA | 0.001 | 1 |
| MTHFD2_14044 | 7 | SNP | non-coding | IVS6 − 135 | | A/G | NA | NA | 0.001 | 1 |
| MTHFD2_14085 | 7 | SNP | non-coding | IVS6 − 94 | rs17009746 | G/T | NA | NA | 0.004 | 0.997 |
| MTHFD2_14253 | 7 | SNP | Nonsynonymous | H280D | | C/G | Benign | Tolerated | 0.001 | 1 |
| MTHFD2_14491 | 7 | SNP | non-coding | IVS7 + 187 | rs844169 | G/T | NA | NA | 0.339 | 0.322 |
| MTHFD2_16475 | 8 | SNP | non-coding | IVS7 − 42 | | T/C | NA | NA | 0.001 | 1 |
| MTHFD2_16635 | 8 | SNP | Synonymous | E336E | | G/A | NA | NA | 0.001 | 1 |
| MTHFD2_16889 | 8 | SNP | non-coding | 3′UTR | | G/A | NA | NA | 0.003 | 0.997 |
| MTHFD2_16903 | 8 | SNP | non-coding | 3′UTR | | C/T | NA | NA | 0.005 | 0.995 |

TABLE S
MTHFR Genetic Variants

| GENE_position | Exon | Type | Function | Location | dB SNP id | Change | PolyPhen | SIFT | MAF | HWE |
|---|---|---|---|---|---|---|---|---|---|---|
| MTHFR_3921 | 2 | SNP | non-coding | 5′-UTR | rs34889587 | C/T | NA | NA | 0.001 | 1 |
| MTHFR_4059 | 2 | SNP | Synonymous | P39P | rs2066470 | C/T | NA | NA | 0.071 | 0.287 |
| MTHFR_4078 | 2 | SNP | Nonsynonymous | R46W | | C/T | Probably | Affected | 0.001 | 1 |
| | | | | | | | Damaging | | | |
| MTHFR_4145 | 2 | SNP | Nonsynonymous | R68Q | rs2066472 | A/G | Possibly | Affected | 0.001 | 0 |
| | | | | | | | Damaging | | | |
| MTHFR_4181 | 2 | SNP | non-coding | IVS2 + 3 | rs1413355 | A/G | NA | NA | 0.001 | 1 |
| MTHFR_4234 | 2 | SNP | non-coding | IVS + 56 | | A/G | NA | NA | 0.001 | 1 |
| MTHFR_5699 | 3 | SNP | Synonymous | D92D | rs45546035 | C/T | NA | NA | 0.001 | 1 |
| MTHFR_5733 | 3 | SNP | Nonsynonymous | D104Y | | G/T | Possibly | Tolerated | 0.001 | 1 |
| | | | | | | | Damaging | | | |
| MTHFR_5840 | 3 | SNP | Synonymous | T139T | rs2066466 | A/G | NA | NA | 0.016 | 0.949 |
| MTHFR_5872 | 3 | SNP | Nonsynonymous | L150P | | C/T | Possibly | Affected | 0.001 | 0 |
| | | | | | | | Damaging | | | |
| MTHFR_6642 | 4 | SNP | non-coding | IVS3 − 95 | | C/T | NA | NA | 0.001 | 0 |
| MTHFR_6651 | 4 | SNP | non-coding | IVS3 − 86 | rs13306567 | C/G | NA | NA | 0.041 | 0.197 |
| MTHFR_6657 | 4 | SNP | non-coding | IVS3 − 80 | | C/T | NA | NA | 0.001 | 0 |
| MTHFR_6658 | 4 | SNP | non-coding | IVS3 − 79 | rs2066471 | A/G | NA | NA | 0.131 | 0.807 |
| MTHFR_6661 | 4 | SNP | non-coding | IVS3 − 76 | rs2066469 | A/G | NA | NA | 0.005 | 0.995 |
| MTHFR_6681 | 4 | indel | non-coding | IVS3 − 56 | | delAG | NA | NA | 0.001 | 1 |
| MTHFR_6774 | 4 | SNP | Synonymous | G171G | | A/C | NA | NA | 0.001 | 1 |
| MTHFR_10738 | 5 | SNP | Nonsynonymous | A222V | rs1801133 | C/T | Benign | Affected | 0.41 | 0.154 |
| MTHFR_10906 | 5 | SNP | non-coding | IVS5 + 53 | | C/T | NA | NA | 0.001 | 1 |
| MTHFR_11656 | 6 | SNP | non-coding | IVS5 − 55 | | C/T | NA | NA | 0.001 | 0 |
| MTHFR_11668 | 6 | SNP | non-coding | IVS5 − 43 | | C/T | NA | NA | 0.001 | 1 |
| MTHFR_11836 | 6 | SNP | Synonymous | A302A | rs13306555 | C/T | NA | NA | 0.001 | 0 |
| MTHFR_11902 | 6 | SNP | Synonymous | N324N | | C/T | NA | NA | 0.001 | 1 |
| MTHFR_12044 | 6 | SNP | non-coding | IVS6 + 83 | rs2066467 | A/G | NA | NA | 0.001 | 1 |
| MTHFR_12190 | 7 | SNP | non-coding | IVS6 − 6 | rs2066464 | A/G | NA | NA | 0.001 | 1 |
| MTHFR_12220 | 7 | SNP | Synonymous | S352S | rs2066462 | C/T | NA | NA | 0.074 | 0.672 |
| MTHFR_12232 | 7 | SNP | Synonymous | K356K | | A/G | NA | NA | 0.001 | 1 |
| MTHFR_12361 | 7 | SNP | non-coding | IVS7 + 31 | rs1994798 | C/T | NA | NA | 0.335 | 0.975 |
| MTHFR_12445 | 8 | SNP | non-coding | IVS7 − 76 | rs12121543 | G/T | NA | NA | 0.153 | 0.634 |
| MTHFR_12618 | 8 | SNP | Nonsynonymous | G422R | rs4557173 | A/G | Probably | Affected | 0.001 | 1 |
| | | | | | | | Damaging | | | |
| MTHFR_12622 | 8 | indel | Frameshift | E423fs | | insG | Frameshift | Frameshift | 0.003 | 0.998 |
| | | frameshift | | | | | | | | |
| MTHFR_12641 | 8 | SNP | Nonsynonymous | E429A | rs1801131 | A/C | Benign | Affected | 0.183 | 0.606 |
| MTHFR_12660 | 8 | SNP | Synonymous | F435F | rs4846051 | C/T | NA | NA | 0.049 | 0.038 |
| MTHFR_13040 | 9 | SNP | Nonsynonymous | R473W | | C/T | Benign | Tolerated | 0.001 | 1 |
| MTHFR_13099 | 9 | SNP | Synonymous | P492P | rs35653697 | A/G | NA | NA | 0.004 | 0.997 |
| MTHFR_13192 | 9 | SNP | non-coding | IVS9 + 39 | rs45515693 | C/T | NA | NA | 0.003 | 0.999 |
| MTHFR_13201 | 9 | SNP | non-coding | IVS9 + 48 | | G/T | NA | NA | 0.002 | 1 |
| MTHFR_14601 | 10 | SNP | non-coding | IVS9 − 80 | rs17375901 | A/G | NA | NA | 0.021 | 0.893 |
| MTHFR_14612 | 10 | SNP | non-coding | IVS9 − 69 | | A/G | NA | NA | 0.001 | 1 |
| MTHFR_14705 | 10 | SNP | Nonsynonymous | R519C | rs45496998 | C/T | Benign | Affected | 0.002 | 0.999 |
| MTHFR_14814 | 10 | SNP | non-coding | IVS10 + 32 | rs45497396 | C/T | NA | NA | 0.002 | 0.999 |
| MTHFR_14817 | 10 | SNP | non-coding | IVS10 + 35 | rs1476413 | A/G | NA | NA | 0.201 | 0.824 |
| MTHFR_16114 | 12 | SNP | non-coding | IVS11 − 48 | rs3818762 | C/G | NA | NA | 0.197 | 0.671 |
| MTHFR_16136 | 12 | SNP | non-coding | IVS11 − 26 | rs45622739 | A/G | NA | NA | 0.003 | 0.998 |
| MTHFR_16170 | 12 | SNP | Synonymous | A587A | | C/T | NA | NA | 0.001 | 1 |
| MTHFR_16190 | 12 | SNP | Nonsynonymous | R594Q | rs2274976 | A/G | Possibly | Tolerated | 0.041 | 0.967 |
| | | | | | | | Damaging | | | |
| MTHFR_16367 | 12 | SNP | Nonsynonymous | T653M | rs35737219 | C/T | Benign | Affected | 0.011 | 0.973 |
| MTHFR_16368 | 12 | SNP | Synonymous | T653T | rs45572531 | A/G | NA | NA | 0.001 | 1 |
| MTHFR_16401 | 12 | SNP | non-coding | 3′UTR | | C/T | NA | NA | 0.001 | 1 |
| MTHFR_16451 | 12 | SNP | non-coding | 3′UTR | | C/T | NA | NA | 0.001 | 1 |

TABLE T
MTHFS Genetic Variants

| GENE_position | Exon | Type | Function | Location | dB SNP id | Change | PolyPhen | SIFT | MAF | HWE |
|---|---|---|---|---|---|---|---|---|---|---|
| MTHFS_8636 | 2 | SNP | Non-coding | IVS1 − 39 | rs16971502 | C/T | NA | NA | 0.066 | 0.308 |
| MTHFS_8808 | 2 | SNP | Nonsynonymous | R84Q | | A/G | Benign | Tolerated | 0.001 | 1 |
| MTHFS_8912 | 2 | SNP | Nonsynonymous | V119L | | C//G | Benign | Tolerated | 0.001 | 1 |
| MTHFS_8957 | 2 | SNP | Non-coding | IVS2 + 21 | | A/G | NA | NA | 0.004 | 0.996 |
| MTHFS_8998 | 2 | SNP | Non-coding | IVS2 + 62 | | A/G | NA | NA | 0.001 | 1 |
| MTHFS_52560 | 3 | SNP | Non-coding | IVS2 − 27 | | C/T | NA | NA | 0.003 | 0.998 |
| MTHFS_52811 | 3 | SNP | Nonsynonymous | T202A | rs8923 | A/G | Benign | Tolerated | 0.061 | 0.187 |
| MTHFS_52878 | 3 | SNP | Non-coding | 3′UTR | | A/G | NA | NA | 0.001 | 1 |
| MTHFS_52902 | 3 | SNP | Non-coding | 3′UTR | | G/T | NA | NA | 0.001 | 1 |

TABLE U
MTR Genetic Variants

| GENE_position | Exon | Type | Function | Location | dB SNP id | Change | PolyPhen | SIFT | MAF | HWE |
|---|---|---|---|---|---|---|---|---|---|---|
| MTR_1357 | 1 | SNP | non-coding | 5′UTR | rs3738547 | C/T | NA | NA | 0.021 | 0.895 |
| MTR_1418 | 1 | SNP | non-coding | 5′UTR | | G/C | NA | NA | 0.001 | 1 |
| MTR_1502 | 1 | SNP | non-coding | IVS1 + 45 | rs10399834 | C/T | NA | NA | 0.006 | 0.99 |
| MTR_9090 | 2 | SNP | non-coding | IVS1 − 58 | | C/T | NA | NA | 0.002 | 0.999 |
| MTR_9268 | 2 | SNP | Nonsynonymous | R52Q | rs12749581 | G/A | Benign | Affected | 0.002 | 0.999 |
| MTR_11857 | 3 | SNP | non-coding | IVS2 − 7 | | G/A | NA | NA | 0.02 | 0.91 |
| MTR_14394 | 4 | SNP | non-coding | IVS3 − 30 | | A/G | NA | NA | 0.001 | 1 |
| MTR_14403 | 4 | SNP | non-coding | IVS3 − 21 | | C/T | NA | NA | 0.016 | 0.056 |
| MTR_14418 | 4 | SNP | non-coding | IVS3 − 6 | rs7526063 | C/T | NA | NA | 0.035 | 0.004 |
| MTR_16239 | 5 | SNP | Synonymous | V142V | | G/C | NA | NA | 0.001 | 1 |
| MTR_18459 | 6 | SNP | Synonymous | T168T | | A/G | NA | NA | 0.001 | 1 |
| MTR_18541 | 6 | SNP | Nonsynonymous | I196V | | A/G | Benign | Tolerated | 0.001 | 1 |
| MTR_21267 | 7 | SNP | non-coding | IVS6 − 57 | | A/G | NA | NA | 0.001 | 1 |
| MTR_21331 | 7 | indel | Frameshift | L206fs | | delT | Frameshift | Frameshift | 0.001 | 1 |
| | | frameshift | | | | | | | | |
| MTR_21414 | 7 | indel | non-coding | IVS7 + 31 | | delCT | NA | NA | 0.001 | 1 |
| MTR_21416 | 7 | indel | non-coding | IVS7 + 33 | | delGTCT | NA | NA | 0.294 | 0.966 |
| MTR_21420 | 7 | indel | non-coding | IVS7 + 37 | | delTTT | NA | NA | 0.001 | 1 |
| MTR_21458 | 7 | SNP | non-coding | IVS7 + 75 | | G/A | NA | NA | 0.001 | 1 |
| MTR_22149 | 8 | SNP | non-coding | IVS7-24 | | A/C | NA | NA | 0.002 | 1 |
| MTR_22214 | 8 | SNP | Synonymous | S237S | | C/T | NA | NA | 0.001 | 1 |
| MTR_22258 | 8 | SNP | Nonsynonymous | E252G | | A/G | Benign | Tolerated | 0.001 | 1 |
| MTR_22315 | 8 | SNP | non-coding | IVS8 + 48 | | T/G | NA | NA | 0.001 | 1 |
| MTR_29761 | 9 | SNP | non-coding | IVS8 − 82 | | A/G | NA | NA | 0.002 | 0.999 |
| MTR_29817 | 9 | SNP | non-coding | IVS8 − 26 | | T/C | NA | NA | 0.001 | 1 |
| MTR_29936 | 9 | SNP | Synonymous | P286P | | C/T | NA | NA | 0.004 | 0.996 |
| MTR_30968 | 10 | SNP | non-coding | IVS9 − 94 | | T/C | NA | NA | 0.002 | 0.999 |
| MTR_31023 | 10 | indel | non-coding | IVS9 − 40 | | insT | NA | NA | 0.444 | 0.542 |
| MTR_32566 | 11 | SNP | Nonsynonymous | D314N | rs2229274 | G/A | Benign | Tolerated | 0.017 | 0.932 |
| MTR_32581 | 11 | SNP | Nonsynonymous | I319V | | A/G | Benign | Tolerated | 0.001 | 1 |
| MTR_32619 | 11 | SNP | Synonymous | I331I | | C/T | NA | NA | 0.001 | 1 |
| MTR_32647 | 11 | SNP | non-coding | IVS11 + 26 | | T/C | NA | NA | 0.001 | 1 |
| MTR_34866 | 12 | indel | non-coding | IVS11 − 48 | | delATA | NA | NA | 0.002 | 0.999 |
| | | | | | | TT | | | | |
| MTR_34894 | 12 | SNP | non-coding | IVS11 − 20 | | T/C | NA | NA | 0.001 | 1 |
| MTR_34951 | 12 | SNP | Nonsynonymous | V345I | | G/A | Benign | Tolerated | 0.001 | 1 |
| MTR_34976 | 12 | SNP | Nonsynonymous | G353A | | G/C | Benign | Tolerated | 0.001 | 1 |
| MTR_37683 | 13 | SNP | non-coding | IVS12 − 8 | | C/T | NA | NA | 0.002 | 0.999 |
| MTR_43970 | 15 | SNP | non-coding | IVS14 − 169 | | A/G | NA | NA | 0.013 | 0.961 |
| MTR_43998 | 15 | SNP | non-coding | IVS14 − 141 | rs60444984 | T/G | NA | NA | 0.001 | 1 |
| MTR_44246 | 15 | SNP | Synonymous | D479D | | C/T | NA | NA | 0.002 | 0.999 |
| MTR_44294 | 15 | SNP | Nonsynonymous | M495I | | G/A | Benign | Tolerated | 0.001 | 1 |
| MTR_44351 | 15 | SNP | non-coding | IVS15 + 27 | rs3820568 | A/G | NA | NA | 0.469 | 0.206 |
| MTR_44409 | 15 | SNP | non-coding | IVS15 + 85 | | A/G | NA | NA | 0.001 | 1 |
| MTR_44426 | 15 | SNP | non-coding | IVS15 + 102 | | C/T | NA | NA | 0.013 | 0.961 |
| MTR_44427 | 15 | SNP | non-coding | IVS15 + 103 | | A/C | NA | NA | 0.022 | 0.884 |
| MTR_44428 | 15 | SNP | non-coding | IVS15 + 104 | | T/C | NA | NA | 0.011 | 0.973 |
| MTR_44457 | 15 | SNP | non-coding | IVS15 + 135 | rs55748381 | A/C | NA | NA | 0.469 | 0.145 |
| MTR_44459 | 15 | SNP | non-coding | IVS15 + 135 | | A/G | NA | NA | 0.001 | 1 |
| MTR_55976 | 16 | SNP | non-coding | IVS15 − 93 | rs6658027 | A/T/C | NA | NA | | NA |
| MTR_55981 | 16 | indel | non-coding | IVS15 − 88 | rs11288788 | delT | NA | NA | 0.469 | 0.443 |
| MTR_56064 | 16 | SNP | non-coding | IVS15 − 5 | | C/T | NA | NA | 0.002 | 1 |
| MTR_56343 | 16 | indel | non-coding | IVS16 + 95 | rs58373128 | insTGA | NA | NA | 0.461 | 0.988 |
| MTR_58139 | 17 | SNP | non-coding | IVS16 − 107 | | A/G | NA | NA | 0.002 | 0.999 |
| MTR_58157 | 17 | SNP | non-coding | IVS16 − 89 | | T/C | NA | NA | 0.001 | 1 |
| MTR_58168 | 17 | SNP | non-coding | IVS16 − 78 | | C/A | NA | NA | 0.002 | 0.999 |
| MTR_58453 | 17 | SNP | non-coding | IVS17 + 91 | rs3901559 | G/T | NA | NA | 0.002 | 0.999 |
| MTR_58464 | 17 | SNP | non-coding | IVS17 + 102 | | C/G | NA | NA | 0.001 | 1 |
| MTR_58650 | 18 | SNP | non-coding | IVS17 − 23 | | A/G | NA | NA | 0.003 | 0.998 |
| MTR_58819 | 18 | SNP | non-coding | IVS18 + 6 | | C/G | NA | NA | 0.001 | 1 |
| MTR_58884 | 18 | SNP | non-coding | IVS18 + 71 | | G/A | NA | NA | 0.002 | 0.999 |
| MTR_65459 | 19 | SNP | non-coding | IVS18 − 99 | | A/G | NA | NA | 0.001 | 1 |
| MTR_65498 | 19 | SNP | non-coding | IVS18 − 60 | | C/T | NA | NA | 0.001 | 0 |
| MTR_66829 | 20 | SNP | non-coding | IVS19 − 21 | rs12078297 | C/T | NA | NA | 0.027 | 0.414 |
| MTR_66915 | 20 | SNP | Synonymous | R703R | | A/C | NA | NA | 0.038 | 0.729 |
| MTR_68099 | 21 | SNP | non-coding | IVS21 + 31 | | T/G | NA | NA | 0.005 | 0.999 |
| MTR_69121 | 22 | SNP | non-coding | IVS21 − 58 | rs12731423 | A/G | NA | NA | 0.002 | 0.999 |
| MTR_80422 | 24 | SNP | non-coding | IVS23 − 29 | | C/G | NA | NA | 0.002 | 0.999 |
| MTR_80489 | 24 | SNP | Nonsynonymous | D838N | | G/A | Benign | Affected | 0.001 | 1 |
| MTR_80586 | 24 | SNP | non-coding | IVS24 + 15 | rs1770449 | T/C | NA | NA | 0.326 | 0 |
| MTR_86396 | 25 | SNP | non-coding | IVS24 − 84 | | A/G | NA | NA | 0.001 | 1 |
| MTR_90872 | 26 | SNP | Synonymous | L901L | | A/G | NA | NA | 0.004 | 0.996 |
| MTR_90925 | 26 | SNP | Nonsynonymous | D919G | rs1805087 | A/G | Benign | Affected | 0.158 | 0.993 |
| MTR_90987 | 26 | SNP | non-coding | IVS26 + 43 | rs2275566 | A/G | NA | NA | 0.293 | 0.998 |
| MTR_92056 | 27 | SNP | Nonsynonymous | G939R | | G/C | Benign | Affected | 0.001 | 1 |
| MTR_94847 | 28 | SNP | non-coding | IVS27 − 59 | | A/G | NA | NA | 0.001 | 1 |
| MTR_94873 | 28 | SNP | non-coding | IVS27 − 33 | | G/A | NA | NA | 0.001 | 1 |
| MTR_94883 | 28 | SNP | non-coding | IVS27 − 23 | | A/T | NA | NA | 0.001 | 1 |
| MTR_95112 | 28 | SNP | non-coding | IVS28 + 51 | | T/C | NA | NA | 0.001 | 1 |
| MTR_96929 | 29 | SNP | Nonsynonymous | R1027W | | C/T | Probably | Affected | 0.002 | 0.999 |
| | | | | | | | Damaging | | | |
| MTR_96991 | 29 | SNP | Synonymous | Y1047Y | | C/T | NA | NA | 0.001 | 1 |
| MTR_96993 | 29 | SNP | Nonsynonymous | A1048V | | C/T | Benign | Affected | 0.001 | 1 |
| MTR_96994 | 29 | SNP | Synonymous | A1048A | rs2229276 | A/G | NA | NA | 0.458 | 0.384 |
| MTR_100033 | 30 | SNP | non-coding | IVS29 − 49 | rs2297965 | A/G | NA | NA | 0.378 | 0.867 |
| MTR_100214 | 30 | SNP | Nonsynonymous | A1113T | | G/A | Benign | Affected | 0.001 | 1 |
| MTR_100303 | 30 | SNP | non-coding | IVS30 + 21 | | C/T | NA | NA | 0.001 | 1 |
| MTR_100307 | 30 | SNP | non-coding | IVS30 + 25 | rs2297964 | G/A | NA | NA | 0.052 | 0.493 |
| MTR_100323 | 30 | SNP | non-coding | IVS30 + 41 | | C/T | NA | NA | 0.001 | 1 |
| MTR_100356 | 30 | SNP | non-coding | IVS30 + 74 | | A/G | NA | NA | 0.001 | 1 |
| MTR_101047 | 31 | SNP | non-coding | IVS30 − 36 | | G/A | NA | NA | 0.001 | 1 |
| MTR_101151 | 31 | SNP | Synonymous | L1158L | | G/A | NA | NA | 0.003 | 0.999 |
| MTR_101154 | 31 | SNP | Synonymous | D1159D | | C/T | NA | NA | 0.001 | 1 |
| MTR_101168 | 31 | SNP | Nonsynonymous | R1164H | rs61736326 | G/A | Benign | Affected | 0.006 | 0 |
| MTR_101169 | 31 | SNP | Synonymous | R1164R | rs12070777 | C/A | NA | NA | 0.458 | 0.308 |
| MTR_101173 | 31 | SNP | Synonymous | L1166L | rs12030699 | C/T | NA | NA | 0.017 | 0.022 |
| MTR_101195 | 31 | SNP | Nonsynonymous | P1173L | | C/T | Probably | Affected | 0.001 | 1 |
| | | | | | | | Damaging | | | |
| MTR_101253 | 31 | SNP | Synonymous | L1192L | rs1131449 | T/C | NA | NA | 0.403 | 0.91 |
| MTR_102720 | 32 | SNP | non-coding | IVS31 − 11 | | G/A | NA | NA | 0.002 | 0.999 |
| MTR_102721 | 32 | SNP | non-coding | IVS31 − 10 | rs41530146 | C/A | NA | NA | 0.005 | 0.995 |
| MTR_102797 | 32 | SNP | Nonsynonymous | N1222S | rs61739582 | A/G | Benign | Affected | 0.01 | 0.98 |
| MTR_102810 | 32 | SNP | Synonymous | K1226K | | A/G | NA | NA | 0.001 | 1 |
| MTR_102858 | 32 | SNP | non-coding | IVS32 + 15 | rs3820571 | T/G | NA | NA | 0.296 | 0.628 |
| MTR_103275 | 33 | SNP | non-coding | IVS32 − 8 | rs12022937 | T/C | NA | NA | 0.077 | 0.024 |
| MTR_103345 | 33 | SNP | Synonymous | P1258P | | C/T | NA | NA | 0.001 | 1 |
| MTR_103422 | 33 | SNP | Synonymous | 3′UTR | rs12058328 | A/G | NA | NA | 0.002 | 0.999 |
| MTR_103481 | 33 | SNP | non-coding | 3′UTR | rs2853522 | C/A | NA | NA | 0.375 | 0.884 |
| MTR_103522 | 33 | SNP | non-coding | 3′UTR | rs11799670 | A/G | NA | NA | 0.056 | 0.924 |
| MTR_103563 | 33 | SNP | non-coding | 3′UTR | | T/C | NA | NA | 0.003 | 0.998 |

TABLE V
SHMT1 Genetic Variants

| GENE_position | Exon | Type | Function | Location | dB SNP id | Change | PolyPhen | SIFT | MAF | HWE |
|---|---|---|---|---|---|---|---|---|---|---|
| SHMT1_8522 | 2 | indel | non-coding | IVS1 − 21 | | delAT | NA | NA | 0.177 | 0.297 |
| SHMT1_8563 | 2 | SNP | Nonsynonymous | M1R | | T/G | Probably | Affected | 0.005 | 0.996 |
| | | | | | | | Damaging | | | |
| SHMT1_8766 | 2 | SNP | non-coding | IVS2 + 109 | | G/A | NA | NA | 0.005 | 0.997 |
| SHMT1_10878 | 3 | SNP | non-coding | IVS3 + 7 | rs2273026 | G/A | NA | NA | 0.167 | 0.015 |
| SHMT1_10881 | 3 | SNP | non-coding | IVS3 + 10 | rs8070162 | T/C | NA | NA | 0.005 | 0.993 |
| SHMT1_16048 | 4 | SNP | non-coding | IVS3 − 55 | rs28630807 | A/C | NA | NA | 0.02 | 0.114 |
| SHMT1_16062 | 4 | SNP | non-coding | IVS3 − 41 | | T/C | NA | NA | 0.001 | 1 |
| SHMT1_16155 | 4 | SNP | Truncation | R99− | | C/T | Truncation | Truncation | 0.001 | 1 |
| SHMT1_16275 | 4 | SNP | non-coding | IVS4 + 57 | | C/T | NA | NA | 0.001 | 1 |
| SHMT1_16276 | 4 | SNP | non-coding | IVS4 + 58 | | G/A | NA | NA | 0.002 | 1 |
| SHMT1_16984 | 5 | SNP | Synonymous | G152G | | G/C | NA | NA | 0.001 | 1 |
| SHMT1_23777 | 6 | SNP | Synonymous | N189N | | C/T | NA | NA | 0.001 | 1 |
| SHMT1_23864 | 6 | SNP | non-coding | IVS6 + 53 | | A/G | NA | NA | 0.001 | 1 |
| SHMT1_23870 | 6 | SNP | non-coding | IVS6 + 59 | | T/C | NA | NA | 0.001 | 1 |
| SHMT1_24219 | 7 | SNP | non-coding | IVS6 − 69 | rs9897954 | C/T | NA | NA | 0.021 | 0.135 |
| SHMT1_24333 | 7 | SNP | Nonsynonymous | K216R | | A/G | Benign | Tolerated | 0.005 | 0.992 |
| SHMT1_24367 | 7 | SNP | Synonymous | A227A | | G/A | NA | NA | 0.001 | 1 |
| SHMT1_24439 | 7 | SNP | Synonymous | V251V | | G/C | NA | NA | 0.005 | 0.995 |
| SHMT1_28845 | 8 | SNP | non-coding | IVS7 − 23 | rs2273028 | C/T | NA | NA | 0.277 | 0.258 |
| SHMT1_28949 | 8 | SNP | Nonsynonymous | G299D | | G/A | Probably | Affected | 0.001 | 1 |
| | | | | | | | Damaging | | | |
| SHMT1_31341 | 9 | SNP | Nonsynonymous | E340Q | rs7215148 | G/C | Benign | Tolerated | 0.001 | 1 |
| SHMT1_31383 | 9 | SNP | non-coding | IVS9 + 6 | | G/A | NA | NA | 0.002 | 1 |
| SHMT1_33829 | 10 | SNP | non-coding | IVS9 − 43 | rs8080285 | A/C | NA | NA | 0.044 | 0.876 |
| SHMT1_33908 | 10 | SNP | Nonsynonymous | R364H | | G/A | Probably | Affected | 0.002 | 1 |
| | | | | | | | Damaging | | | |
| SHMT1_34047 | 10 | SNP | non-coding | IVS10 + 59 | rs12937300 | A/G | NA | NA | 0.207 | 0.54 |
| SHMT1_35165 | 11 | SNP | Synonymous | S394S | | C/T | NA | NA | 0.001 | 1 |
| SHMT1_35286 | 11 | SNP | non-coding | IVS11 + 21 | rs6502648 | G/T | NA | NA | 0.034 | 0.749 |
| SHMT1_35339 | 11 | SNP | non-coding | IVS11 + 74 | rs17806333 | A/G | NA | NA | 0.006 | 0.99 |
| SHMT1_35712 | 12 | SNP | Truncation | Y457− | | C/G | Truncation | Truncation | 0.003 | 0.998 |
| SHMT1_35721 | 12 | SNP | Synonymous | A460A | | C/T | NA | NA | 0.008 | 0.99 |
| SHMT1_35761 | 12 | SNP | Nonsynonymous | L474F | rs1979277 | C/T | Benign | Affected | 0.233 | 0.299 |
| SHMT1_35840 | 12 | SNP | non-coding | 3′UTR | rs3783 | C/G | NA | NA | 0.216 | 0.555 |
| SHMT1_35845 | 12 | SNP | non-coding | 3′UTR | | C/T | NA | NA | 0.015 | 0.965 |
| SHMT1_35859 | 12 | SNP | non-coding | 3′UTR | rs1979276 | C/T | NA | NA | 0.28 | 0.095 |

TABLE W
SHMT2 Genetic Variants

| GENE_position | Exon | Type | Function | Location | dB SNP id | Change | PolyPhen | SIFT | MAF | HWE |
|---|---|---|---|---|---|---|---|---|---|---|
| SHMT2_968 | 1 | SNP | non-coding | 5′UTR | rs28365863 | G/A | NA | NA | 0.006 | 0.99 |
| SHMT2_2150 | 2 | SNP | Nonsynonymous | S50L | | C/T | Benign | Tolerated | 0.007 | 0.987 |
| SHMT2_2151 | 2 | SNP | Synonymous | S50S | | G/A | NA | NA | 0.001 | 1 |
| SHMT2_2691 | 3 | SNP | non-coding | IVS2 − 22 | | A/G | NA | NA | 0.002 | 0.999 |
| SHMT2_2816 | 3 | SNP | non-coding | IVS3 + 24 | | G/A | NA | NA | 0.002 | 0.999 |
| SHMT2_3134 | 4 | SNP | Synonymous | P167P | | C/T | NA | NA | 0.001 | 1 |
| SHMT2_3157 | 4 | SNP | non-coding | IVS4 + 12 | | T/A | NA | NA | 0.001 | 1 |
| SHMT2_3225 | 4 | SNP | non-coding | IVS4 + 80 | | G/A | NA | NA | 0.001 | 1 |
| SHMT2_3399 | 5 | SNP | non-coding | IVS4 − 44 | | G/A | NA | NA | 0.013 | 0 |
| SHMT2_3467 | 5 | SNP | Synonymous | D179D | rs11557166 | C/T | NA | NA | 0.007 | 0 |
| SHMT2_3602 | 5 | SNP | non-coding | IVS5 + 78 | | G/T | NA | NA | 0.001 | 1 |
| SHMT2_3604 | 5 | SNP | non-coding | IVS5 + 80 | | C/T | NA | NA | 0.001 | 1 |
| SHMT2_3696 | 6 | SNP | Synonymous | G202G | | C/A | NA | NA | 0.001 | 1 |
| SHMT2_3740 | 6 | SNP | Nonsynonymous | R217Q | | G/A | Benign | Affected | 0.001 | 1 |
| SHMT2_3764 | 6 | SNP | Nonsynonymous | T225I | | C/T | Benign | Tolerated | 0.001 | 1 |
| SHMT2_3821 | 6 | indel | non-coding | IVS6 + 14 | | delG | NA | NA | 0.001 | 1 |
| SHMT2_3882 | 7 | SNP | non-coding | IVS6 − 54 | | G/T | NA | NA | 0.001 | 1 |
| SHMT2_3893 | 7 | SNP | non-coding | IVS6 − 43 | | C/T | NA | NA | 0.008 | 0 |
| SHMT2_4016 | 7 | SNP | Synonymous | S266S | rs2229715 | G/A | NA | NA | 0.001 | 1 |
| SHMT2_4023 | 7 | SNP | Nonsynonymous | K269E | | A/G | Benign | Tolerated | 0.001 | 1 |
| SHMT2_4031 | 7 | SNP | Synonymous | A271A | rs2229716 | G/A | NA | NA | 0.018 | 0.922 |
| SHMT2_4038 | 7 | SNP | Nonsynonymous | V274I | | G/A | Benign | Affected | 0.001 | 1 |
| SHMT2_4373 | 8 | indel | non-coding | IVS7 − 39 | | delCTT | NA | NA | 0.001 | 1 |
| SHMT2_4523 | 8 | SNP | Synonymous | L323L | rs2229717 | G/T | NA | NA | 0.058 | 0.485 |
| SHMT2_4974 | 10 | SNP | non-coding | IVS9 − 7 | | A/G | NA | NA | 0.013 | 0.962 |
| SHMT2_5147 | 10 | SNP | non-coding | IVS10 + 11 | | G/A | NA | NA | 0.002 | 0.999 |
| SHMT2_5166 | 10 | SNP | non-coding | IVS10 + 30 | rs34095989 | G/A | NA | NA | 0.289 | 0.034 |
| SHMT2_5227 | 11 | SNP | non-coding | IVS10 − 8 | | C/T | NA | NA | 0.001 | 1 |
| SHMT2_5265 | 11 | SNP | Nonsynonymous | R437H | | G/A | Benign | Tolerated | 0.001 | 1 |
| SHMT2_5520 | 12 | SNP | Nonsynonymous | R481H | | G/A | Benign | Affected | 0.005 | 0.993 |
| SHMT2_5541 | 12 | SNP | Nonsynonymous | R488Q | | G/A | Benign | Tolerated | 0.001 | 1 |
| SHMT2_5663 | 12 | SNP | non-coding | 3′UTR | | G/A | NA | NA | 0.001 | 1 |

TABLE X
TYMS Genetic Variants

| GENE_position | Exon | Type | Function | Location | dB SNP id | Change | PolyPhen | SIFT | MAF | HWE |
|---|---|---|---|---|---|---|---|---|---|---|
| TYMS_2982 | 2 | indel | non-coding | IVS1 − 56 | | delTTG | NA | NA | 0.008 | 0.983 |
| | | | | | | GATG | | | | |
| TYMS_5475 | 3 | SNP | non-coding | IVS2 − 68 | | C/T | NA | NA | 0.007 | 0 |
| TYMS_5476 | 3 | SNP | non-coding | IVS2 − 67 | | G/A | NA | NA | 0.002 | 0.999 |
| TYMS_5500 | 3 | SNP | non-coding | IVS2 − 43 | rs1001761 | G/A | NA | NA | 0.438 | 0.352 |
| TYMS_5532 | 3 | SNP | non-coding | IVS2 − 11 | rs11873890 | A/G | NA | NA | 0.008 | 0 |
| TYMS_5644 | 3 | SNP | Synonymous | E127E | rs3786362 | A/G | NA | NA | 0.042 | 0.424 |
| TYMS_5767 | 3 | SNP | non-coding | IVS3 + 50 | rs2612095 | T/C | NA | NA | 0.438 | 0.429 |
| TYMS_12530 | 4 | SNP | Synonymous | P172P | | T/C | NA | NA | 0.001 | 0.999 |
| TYMS_12581 | 4 | SNP | non-coding | IVS4 + 11 | | C/A | NA | NA | 0.001 | 0.999 |
| TYMS_12584 | 4 | SNP | non-coding | IVS4 + 14 | rs35710611 | C/T | NA | NA | 0.017 | 0 |
| TYMS_14015 | 5 | SNP | non-coding | IVS4 − 74 | | C/T | NA | NA | 0.005 | 0 |
| TYMS_14018 | 5 | SNP | non-coding | IVS4 − 71 | | G/A | NA | NA | 0.001 | 0.999 |
| TYMS_14201 | 5 | SNP | Synonymous | V223V | | G/A | NA | NA | 0.001 | 0.999 |
| TYMS_14277 | 5 | SNP | non-coding | IVS5 + 13 | | G/A | NA | NA | 0.001 | 0.999 |
| TYMS_14285 | 5 | SNP | non-coding | IVS5 + 21 | rs3826626 | T/C | NA | NA | 0.045 | 0.005 |
| TYMS_14387 | 5 | indel | non-coding | IVS5 + 123 | | delTTA | NA | NA | 0.001 | 0 |
| | | | | | | AG | | | | |
| TYMS_14392 | 5 | SNP | non-coding | IVS5 + 128 | rs2612098 | A/C | NA | NA | 0.438 | 0.561 |
| TYMS_14770 | 6 | SNP | non-coding | IVS5 − 7 | | T/G | NA | NA | 0.001 | 1 |
| TYMS_14917 | 6 | SNP | non-coding | IVS6 + 69 | rs2853536 | C/T | NA | NA | 0.341 | 0.288 |
| TYMS_16189 | 7 | SNP | non-coding | IVS6 − 68 | rs1059394 | C/T | NA | NA | 0.354 | 0.175 |
| TYMS_16198 | 7 | SNP | non-coding | IVS6 − 59 | | G/A | NA | NA | 0.001 | 1 |
| TYMS_16233 | 7 | SNP | non-coding | IVS6 − 24 | rs1059393 | A/G | NA | NA | 0.079 | 0.297 |
| TYMS_16413 | 7 | SNP | non-coding | 3′UTR | rs699517 | C/T | NA | NA | 0.353 | 0.202 |
| TYMS_16483 | 7 | SNP | non-coding | 3′UTR | rs2790 | A/G | NA | NA | 0.24 | 0.985 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the disclosure as described herein may be employed in practicing the present invention. It is intended that the following claims define the scope of the present invention and that methods and structures within the scope of these claims and their equivalents be covered thereby.

### EXAMPLES

### Example 1

### Prevalence of Folate-Remedial MTHFR Enzyme Variants in Humans

The prevalence of folate-remediable MTHFR enzyme variants from a large population to determine the incidence and impact of low frequency variation and explore the phenomenon of vitamin remediation. From over 500 individuals, 14 different non-synonymous substitutions were identified, 5 of which impaired enzyme function. While all deleterious alleles were at least somewhat folate responsive, 4 of the 5 mutant proteins could be fully restored to normal levels by elevating intracellular folate levels.

Methods

DNA Sample Population. DNA samples were from the Coriell Institute Cell Repository (Camden, N.J., USA).

MTHFR Exon Sequencing. 11 MTHFR coding exons were sequenced in the above samples by PCR sequencing using primer pairs commercially available from the Variant SeqR product line (Applied Biosystems, Foster City, Calif.) and according to the protocols supplied. The exon regions sequenced corresponded to NCBI MTHFR reference sequences for mRNA (NM_{—}005957) and the corresponding protein (NP 005958) of 656 amino acids. Sequencing amplicon and probe information is available at http://www.ncbi.nlm.nihcov/genome/probe for the following target amplicons:

Exon 1 (RSA000045684); Exon 2 (RSA000045680); Exon 3 (RSA000577249); Exon 4 (RSA000045678); Exon 5 (RSA000045676); Exon 6 (RSAOO1 308795); Exon 7 (RSAOO1 253193); Exon 8 (RSA000045669); Exon 9 (RSA000580767); Exon 10 (RSA 000580766); Exon 11 (RSA000580765, RSA000027240). Only the portion of exon 11 that spanned the coding region was sequenced. To ensure high confidence in base-calling, only high-quality reads were used for analysis (average QV scores >40 for the region that spanned the target exon; all exons were covered by double-strand reads). Based on these filtering criteria, success rates ranged from 89.9% to 95% for each exon (see Table I). All sequence information was analyzed using the SeqScape software suite (Applied Biosystems). As a quality control measure, a subset of base calls were directly verified by TaqMan (Applied Biosystems) allelic-discrimination assays and compared with publicly available genotype data as described below.

Plasmids. Plasmid phMTHFR, which carries the 5′-end HA (hemagglutinin A) epitope-tagged human MTHFR open reading frame (reference protein sequence NP_{—}005948) under the control of the inducible yeast GAL1 promoter and the URA3 selectable marker, was a generous gift of Warren Kruger (Shan et al., 1999, supra). This plasmid served as the backbone to reconstruct all MTHFR variants by site-directed mutagenesis using the QuikChange kit (Stratagene): Integrating plasmids containing galactose-inducible MTHFR variants were created by PCR cloning the fragment containing URA3, the GAL1 promoter and MTHFR coding region from the phMTHFR-based plasmid into pHOpoly-HO (Voth et al., 2001, Nucleic Acids Res. 29:e59), which enables targeted integration of this cassette at the HO locus.

Strains. All haploid yeast strains were MATa his3 leu2 ura3 lys2 in the S288c background (Brachman et al, 1998, Yeast 14:115-32). MATa/MAT□ diploid strains were created by mating isogenic MATa and MAT□ strains. fol3Δ::KanMX and fol3Δ::KanMX met13Δ::KanMX strains were obtained by standard mating/sporulation techniques using strains from the S. cerevisiae gene-knockout collection (Invitrogen). Diploids (homozygous or heterozygous for MTHFR variants) were created by mating fol3::KanMX met13Δ::KanMX haploids that each contain an integrated version of the GAL1:MTHFR variant cassette.

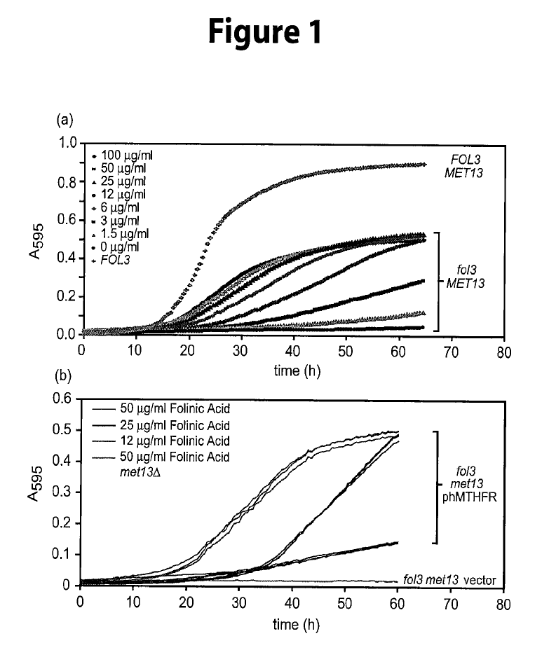

Growth Conditions. Synthetic growth media lacking folate was minimal media (Sherman, 2002, Genetics & Molecular BioL, eds. Guthrie and Fink (Academic, New York), pp. 3-41) with Yeast Nitrogen Base without Vitamins (Qbiogene), and all vitamins except folate added back individually. All fol3Δ::KanMX cells were supplemented with 50 ug/ml folinic acid (Sigma). For kinetic growth measurements, fol3Δ::KanMX met13Δ::KanMX cells were transformed with GAL1 promoter-driven MTHFR variants and grown to log phase in synthetic galactose medium (2% galactose, 0.1% glucose) supplemented with folinic acid (50 ug/ml) and methionine (20 ug/ml). Cells were washed 3 times and aliquoted into 96-well plates containing fresh galactose media with varying amounts of folinic acid, but lacking methionine. The volume per well was 200 ul with a starting cell density of OD=0.01. Absorbance was tracked every 15-30 minutes for at least 60 hours in a Tecan GENios plate reader at 30° C. with no shaking. MET13 cells used in FIG. 1a were treated the same way except that all growth was in the absence of methionine.

MTHFR enzyme activity assay. The assay, which measures the reverse reaction of that catalyzed by MTHFR under physiological conditions, was as described (Shan et al, 1999, supra) with the following modifications: Yeast extracts were created by bead lysis of 40 CD595 cell equivalents (fol3Δ met13Δ cells supplemented with folinic acid and methionine as above) in 350 ul of Lysis Buffer (100 mM Sucrose, 50 mM KHPO₄ (pH 6.3), protease inhibitor cocktail). Extracts were clarified by a brief microcentrifugation, and 10-200 ug of extract used to determine the linear range of activity. Radiolabeled substrate (5-[¹⁴C]MeTHF) was from GE Healthcare Life Sciences. For heat treatment, the reaction mixes without 5-[¹⁴C]MeTHF were heated to 55° C. for the indicated times at which point 5-[¹⁴C]MeTHF was added back and the reaction proceeded.

MTHFR Immunoblot analysis. 10 CD cell equivalents (fol3Δ met13Δ cells supplemented with folinic acid and methionine as above) were extracted in 200 ul 0.1 M NaOH for 15 mm. 50 ul SDS sample buffer (0.5M Tris 6.8, 0.4% SDS) was added to supernatants, which were then boiled, clarified and subject to SDS-PAGE. HA-tagged MTHFR variants were detected on a LI-COR Infrared Imager. Mouse monoclonal anti-HA antibody was from Sigma. Yeast 3-Phosphoglycerate kinase (Pgklp), a loading control, was detected by mouse antibodies generously donated by Jeremy Thorner (University of California, Berkeley, Calif.).

Results

MTI-f FR variants in humans. The entire coding region of human MTHFR was sequenced by amplifying the coding portion in each of 11 exons from 564 individuals of diverse ethnicities. The lengths of the coding regions, the number of alleles interrogated and all nonsynonymous substitutions are listed in Table 4. In all, 2,081,106 bp of coding DNA, and sampled every exon to a depth of over 1,000 alleles were analyzed. These data revealed 14 nonsynonymous changes, 11 of which show a minor allele frequency (MAF)<1%, with 7 alleles seen only once. Some low-frequency alleles were seen previously (see Table 4). The number of low-frequency nonsynonymous substitutions was in good agreement with other studies that sampled deeply into random populations (Martin et al., 2006, Pharmacogenet Genomics 16:265-77; Livingston, 2004, Genome Res 14:1821-31; Glatt et al., 2001, Nat. Genet. 27:435-38). In addition, 3 well-studied common substitutions were observed that displayed the expected global population frequencies (A222V—29.3%, E429A—23.6%, R594Q—4.4%).

As a quality-control check on the accuracy of the base-calling, 8 variants (including 4 singletons) were reanalyzed by TaqMan allelic-discrimination assays in 100 samples that were independently PCR-amplified and saw 100% concordance of the data. Furthermore, population genotyping data from the Environmental Genome Project (http:/fwww.niehs.nih.gov/envqenom/) and Perlegen (Mountain View, Calif.), which both used Coriell samples that overlap some in this study (dbSNP build 127) were in concordance in 814 of 817 (99.6%) genotype calls. For two of the three discordant loci, our sequence data were unambiguous and appeared correct.

Complete coding region sequences were obtained for 480 individuals. Eighteen (4%) were carriers of a low-frequency nonsynonymous variant. Significantly, the combination of the 3 common polymorphisms (A222V, E429A and R594Q) with the range of the low frequency changes led to a great deal of individual heterogeneity. Twenty-eight different nonsynonymous genotypes were observed in this group whose haplotype, in most cases, could not be deduced from the data.

MTHFR folate interaction in vivo. Because the clinical significance of genetic variants lies in their functional consequence, all nonsynonymous changes were tested for their effect on MTHFR function, and importantly, whether or not impaired alleles displayed folate-responsiveness. Folate auxotrophy (fol3) was introduced into a met13 strain, allowing titration of intracellular folate concentrations by varying folinic acid in the growth media. Folinic acid (5-formyl-tetrahydrofolate) can be metabolized in yeast to methenyl-tetrahydrofolate, which in turn can be converted to other folate coenzymes (Cherest et al. (2000) J. Biol. Chem. 275:14056-63). In this way, human MTHFR functionality (growth in the absence of methionine) was measured as a function of increasingly limiting cellular folate status.

Under these conditions, folinic acid supplementation above 50 ug/ml did not confer any significant growth advantage (FIG. 1a). However, at concentrations below 50 ug/ml, growth clearly correlated with available folinic acid in the medium. Thus intracellular folate levels were rate-limiting in this range. When compared to growth of FOL3 cells, folinic acid supplementation did not completely compensate for lack of endogenous folate biosynthesis. However, this gap was mostly reflected in the density at which cells entered stationary phase rather than growth rate, perhaps reflecting limitations in folinic acid uptake, or in the utilization of folinic acid as the sole folate source.

The ability of human MTHFR to complement fol3 met13 cells was a function of folinic acid supplementation in the media (FIG. 1b). As for folate supplementation, expression of human MTHFR from the GAL1 promoter did not completely compensate for loss of Met13p (compare FIG. 1b with FOL3 MET13 cells at equivalent folate doses in FIG. 1a). Thus, below 50 ug/ml folinic acid, both folate and MTHFR were rate-limiting for growth, allowing even subtle changes in MTHFR activity to be reflected in the growth readout. Note that folinic acid supplementation above 50 ug/ml did not confer a significant growth advantage to cells expressing either the endogenous yeast MTHFR (MET13, FIG. 1a) or the major human allele (FIG. 1b), but was beneficial for impaired alleles of MTHFR (see below).

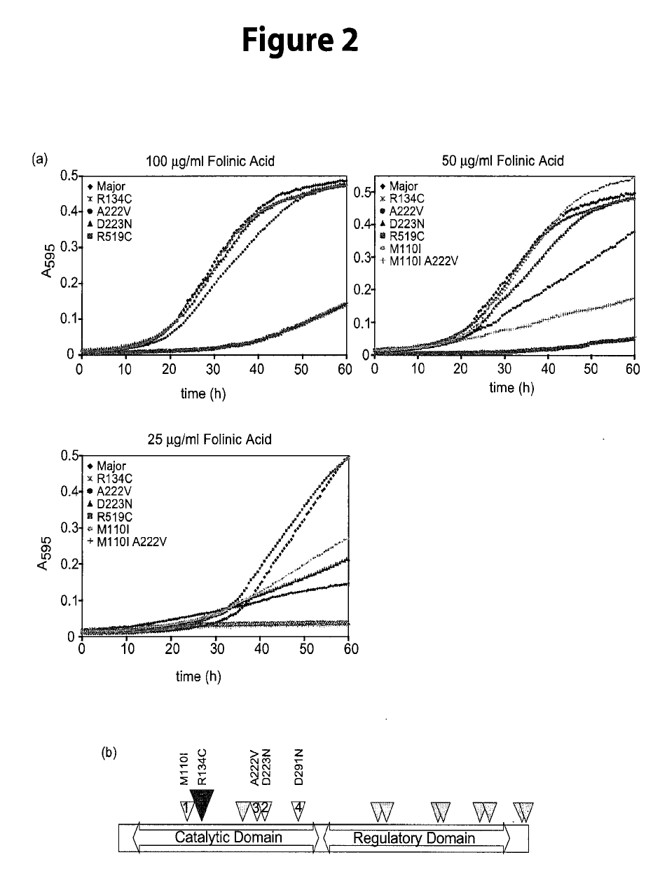

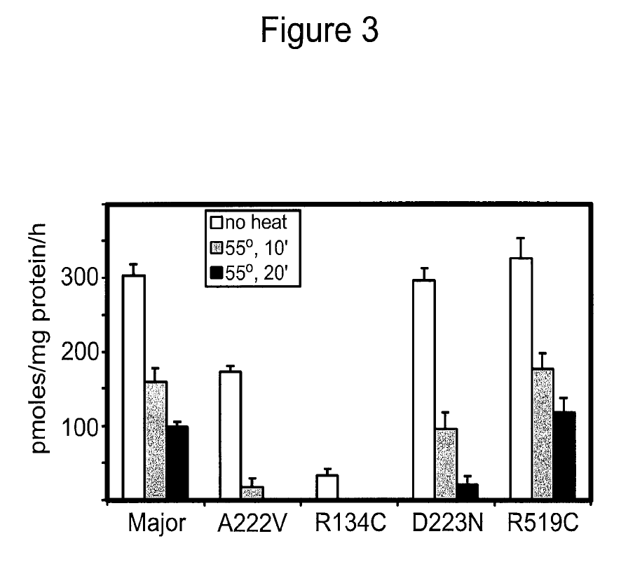

Functional impact of MTHFR variants. Five nonsynonymous alleles tested over a range of folate concentrations illustrated the range of functional effects observed (FIG. 2a). There was nearly complete restoration of function of the A222V variant at 100 ug/ml folinic acid and significantly less activity (relative to the major allele) at a four-fold lower level of supplementation (25 ug/ml). Thus, under these conditions the known folate remediability of the A222V defect was recapitulated. The exact intracellular concentrations of reduced folates in yeast under these conditions was unknown. Nevertheless, the behavior of the A222V allele effectively calibrated the intracellular concentrations in yeast and human cells. The A222V enzyme has approximately 50% the intrinsic activity of common allele (Martin, 2006, Pharmacogenet Genomics 16:265-77; Rozen, 1997, Thromb. Haemost, 78:523-26) and 50% reduction in growth rate was observed at 50 p g/ml folate supplementation. Furthermore, the same 50% drop in A222V enzyme activity in cell-free assays from cells grown at 50 ug/ml folinic acid was observed (FIG. 3, below). Thus, the behavior of A222V in yeast recapitulated its behavior in human cells.

Four low-frequency alleles were tested in the same way (FIG. 2a). R519C appeared benign since growth was unaffected at all folate concentrations. R134C was severely impaired at all folate concentrations, though activity was somewhat folate-responsive. The D223N and MilOl alleles displayed folate-remedial activity similar to A222V (though less severely impaired) in that growth was similar to the major allele at, or above, 50 ug/ml folinic acid, but functioned poorly below 50 ug/ml folinic acid.

The MTHFR enzyme has an N-terminal catalytic domain and a C-terminal regulatory domain, which binds the allostenc inhibitor S-adenosylmethionine (AdoMet; Sumner et al., 1986, J. Biol. Chem, 261: 7697-7700). Of the 6 alleles that fell within the catalytic domain (M110I, R134C, H213R, A222V, D223N and D291N), only H213R was benign (FIG. 2b). M110I, A222V, D223N and D291N displayed folate-remedial behavior in that these enzyme variants were similar to the major allele at higher concentrations of folate supplementation (50-200 ug/ml folinic acid), but were considerably weakened as folate became more rate-limiting. The R134C variant never approached the capacity of the major allele to support growth at any level of folate supplementation and hence was classified as a responsive, but not a remedial allele. All substitutions within the regulatory domain (from G422R through T653M) behaved similarly to the major allele (FIG. 2b).

Synergistic interactions between amino acid substitutions. The distribution of variants implied the existence of compound alleles containing two (or more) substitutions. Therefore several compound alleles (based upon their occurrence in individual samples) were created to test whether allele combinations lead to synergistic or suppressive effects. For A222V combinations with common variants (A222V E429A and A222V RS940), minor allele homozygotes were observed for at least one of the alleles arid therefore are sure that such variants exist. However, for the low frequency variants, both the A222V variant and the novel variants always occurred as heterozygotes, Since the haplotype is unknown, these individuals could harbor either the two single substitution alleles or a compound allele. Therefore all possible double-substitution alleles were created and tested their function (eg. M110I A222V, FIG. 2a). At the two folinic acid concentrations tested, the M110I A222V variant functioned more poorly than the sum of the individual alleles, indicating synergistic defects in compound alleles. At 50 ug/ml folinic acid, the M110I variant was nearly indistinguishable from the major allele, yet it significantly enhanced the A222V defect. For all combinations tested, alleles that affected function individually (M110I and D291N) synergized when combined with A222V, whereas benign changes did not enhance the A222V defect.

Biochemical assays recapitulated in in vivo function. To evaluate the reliability of the growth assay, cell-free MTHFR enzyme assays were performed for all variants in crude yeast lysates (see Materials and Methods). In addition to measuring specific activity, variants were tested for thermolability (a measure of enzyme stability) by heat treatment at 55° C. for various times. There was a good correlation between intrinsic activity and growth rate (FIG. 3; compare the activities of non heat-treated samples for the major MTHFR allele, A222V and R134C with the growth curves in FIG. 2). Again, the A222V variant displayed approximately 50% of the enzymatic activity of the major allele. As in the growth assay, the R519C variant exhibited similar activity to the major allele and was representative of all changes in the regulatory domain including the common E429A variant (data not shown). Although there have been reports that E429A affects enzyme function, our data agreed with others that this change was benign.

The A222V mutant enzyme is less stable and more thermolabile than the major form (Guenther et al. 1999, Nat. Struct. Biol. 6:359-65; Yamada et al. 2001, Proc. Natl. Acad Sci. 98:14853-58) and folate remediation of this variant is thought to occur by promoting stabilization of the protein. Under the conditions used here (55° C., 20 m), A222V lost nearly all activity while the major allele retained about 30% of its original activity, in agreement with previous studies. The novel D223N allele also displayed increased thermolability that may similarly explain folate-remediability in this case, although the enzyme defect was not as great.

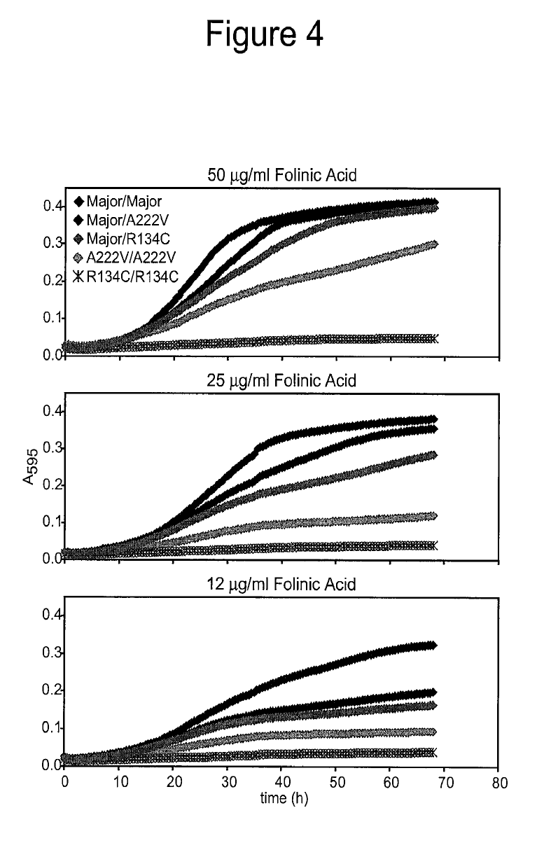

Heterozygote phenotypes. Since low frequency alleles usually occur as heterozygotes, their significance tends to be dismissed. To understand better the functional significance of heterozygosity of MTHFR alleles, diploid yeast with two copies of human MTHFR were created by mating haploid strains that each have either the same allele expressed from an integrated expression cassette (homozygotes) or different alleles to create heterozygotes (see Methods). As above, these strains were tested for growth as a function of folate supplementation (FIG. 4). Heterozygotes displayed a growth phenotype in this assay that was exacerbated under conditions of limiting folate, indicating that the reduced function alleles were codominant with wild type.

Cellular MTHFR activity as measured in the growth assay appeared to reflect additive effects of alleles. Furthermore, additional experiments with hemizygotes (diploids with a single integrated expressed allele; data not shown) demonstrated that the formation of heterodimers between major and minor alleles in heterozyotes offered little or no rescue of mutant alleles. For example, diploid MTHFR major allele/null cells (hemizygotes) behaved similarly to major allele/R134C heterozygotes under all conditions, and similarly to major allele/A222V heterozygotes in low folate media (where A222V is inactivated). Thus, the phenotypic contribution of deleterious alleles in heterozygote cells was easily observed, raising the possibility of more widespread phenotypic consequences from heterozygosity in the human genome.

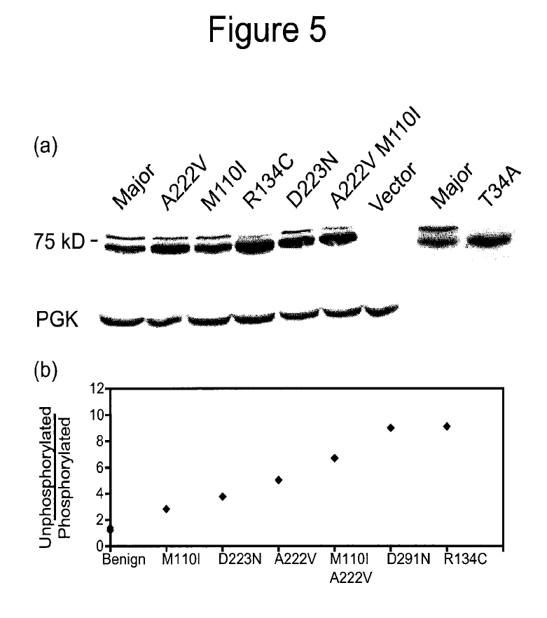

Modification of MTHFR variants in yeast by phosphorylation. The abundance of MTHFR variant proteins was determined by immunoblotting using antibodies directed against the N-terminal hemagglutinin A (HA) epitope tag (FIG. 5a). In all samples, the protein ran as a doublet of approximately 72 kD and 78 kD. This pattern closely resembled that observed for human MTHFR expressed in insect cells, where the upper band represents MTHFR multiply-phosphorylated near the N-terminus. Phosphorylation of MTHFR in insect cells is dependent on a threonine residue at position 34 and substitution of this threonine to alanine (T34A) results in an enzyme that is unable to be phosphorylated. This mutation had the same effect on human MTHFR expressed in S. cerevisiae and indicated that, as in insect cells, the upper band was phosphorylated MTHFR (FIG. 5a).

The role of phosphorylation of MTHFR is suggested to be involved in negative regulation. In support of this hypothesis, the phosphorylation pattern observed here directly correlated with cellular MTHFR activity. Specifically, the ratio of the abundance of the unphosphorylated:phosphorylated forms increased with decreasing activity (FIG. 5b). Interestingly, the overall abundance of all variants (phosphorylated plus unphosphorylated forms) did not appear to be strikingly different. This might not be expected if deleterious substitutions affected intrinsic enzyme stability, unless other factors are involved in determining protein levels.

All functionally impaired alleles clustered in the N-terminal, catalytic half of MTHFR which contains the folate and FAD binding sites. On the other hand, 8 nonsynonymous substitutions in the C-terminal regulatory domain of MTHFR were identified and all 8 appeared benign in both the complementation and cell-free enzyme assays. Furthermore, no synergy was seen between regulatory domain substitutions and A222V in compound alleles (FIG. 2). Either these alterations were neutral, as has been reported for E429A, or the assay was insensitive to their defect. This finding however was consistent with the observation that most mutations in MTHFR that result in severe clinical phenotypes occur in the catalytic domain (http://www.hgmd.cf.ac.uk!ac/index.nhP). The regulatory domain has been proposed to play a role in stabilization of the catalytic domain. If so, this role may be somewhat tolerant to amino acid substitutions and may explain how a chimeric MTHFR composed of the S. cerevisiae N-terminal domain fused to the Arabidopsis C-terminal domain (equivalent to approximately 50 nonsynonymous substitutions of the yeast enzyme in the regulatory domain) does not harm enzyme activity. It should be noted that it has been previously reported that the common RS940 variant in the C-terminal domain affected enzyme activity when expressed in COS-1 cells. This change appeared benign, however, in cell-based and cell-free assays of the enzyme expressed in yeast. Although the reason for this discrepancy is unclear, it may be reflective of the host expression system since these authors observed only a single species of MTHFR (unknown phosphorylation status) in their immunoblot analyses.

The phenotypes of heterozygotes. The behavior of diploid yeast heterozygous for functionally impaired MTHFR alleles demonstrated that heterozygote phenotypes were clearly observable, especially under conditions of limiting folate (FIG. 4). The appearance of phenotypes in heterozygotes was significant since most genetic variation occurs as heterozygosity and low frequency alleles exist primarily as heterozygotes in the population. This result is consistent with the observations that cellular MTHFR activity in lymphocyte extracts is directly correlated with genotype: individuals heterozygous for A222V (NV) have approximately 65% of the total activity seen for major allele (NA) homozygotes, where A222V homozygotes (VN) retain 30% of the activity of A/A homozygotes, In a recent study examining the full spectrum of alleles in the adipokine ANGPTL4, which affects serum triglyceride levels, heterozygosity for the nonsynonymous E4OK allele was significantly associated with lower plasma triglyceride levels. Thus, cases in which heterozygosity is phenotypically detectable increases the significance of the contribution of low frequency variants since there can be orders of magnitude more carriers than homozygotes. Note that heterozygote phenotypes was observed under conditions in which MTHFR activity was rate-limiting for cell growth. Whether or not enzymatic steps are rate-limiting in a particular pathway in humans depends on both genetic and environmental factors.

Mutations and MTHFR phosphorylation and abundance. Folate remediation of nonsynonymous changes in the catalytic domain may occur by protein stabilization (as for A222V) or by overcoming other aspects of molecular function such as cofactor Km. At least one deleterious allele, D223N, showed increased thermolability (FIG. 3) analogous to A222V, which argued for a stability defect. The hypothesis that folate-remedial alleles of MTHFR are those in which a folate species stabilizes unstable forms of the enzyme would suggest that the level of MTHFR protein be proportional to intrinsic activity of the variants, as has been suggested. However, our observations indicated that while phosphorylation status correlated with enzyme activity (FIG. 5), the overall abundance (phosphorylated plus unphosphorylated forms) did not appear to change strikingly (within a two-fold range). It is unlikely that phosphorylated MTHFR is the active form of the enzyme since previous studies have demonstrated an inhibitory effect of phosphorylation on intrinsic activity. Consistent with this, the behavior of the non-phosphorylatable T34A variant in both the growth and enzyme assays was similar to that of the major allele (data not shown). Furthermore, while low intracellular folate levels decrease MTHFR stability (as measured by abundance), this effect is not enhanced in variants that impair function. Because these results are at variance with the expected protein destabilization of deleterious changes, it was deduced there must be a compensatory regulatory response that is currently under investigation. In this way the activity of variants could be strikingly different (FIG. 2), whereas the overall protein abundance may not be (FIG. 5). While our results are consistent with feedback regulation by phosphorylation, the role of phosphorylation in turnover is unknown. In this vein, it will be interesting to determine the effect of the T34A change in combination with other impaired alleles.

The Folate/Homocysteine Metabolic Pathway

The folate/homocysteine metabolic pathway is relevant to the etiology of neural tube defects (NTDs) and other adverse pregnancy outcomes for which folate supplementation has been demonstrated to be preventative and for which elevated plasma homocysteino levels contribute to increased risk. The folate and homocysteine metabolism pathway is linked via the Methionine Synthase reaction, and marginal folate deficiencies in cell cultures, animal model systems and in humans impair homocysteine remethylation (see, for example, Stover P J. 2004. Physiology of folate and vitamin B₁₂ in health and disease. Nutr Rev 62:S3-12). Homocysteine is a hypothesized risk factor for NTDs (see, for example, Mills et. al., 1995. Homocysteine metabolism in pregnancies complicated by neural tube defects. Lancet 345:149-1151). Folate deficiency also impairs methylation mediated by S-adenosyl-methionine (SAM; see, for example, Stover, supra), which is an allosteric inhibitor of both MTHFR and CBS (see, for example, Kraus et al., 1999. Cystathionine-3-synthase mutations in homocystinuria. Hum Mut 13:362-375; Daubner et al., 1982. In Flavins and Flavoproteins, eds. Massey, V. & Williams, C. H (Elsevier, New York), pp. 165-172). Furthermore, elevations in the Sadenosyl-homocysteine:S-adenosyl-methiofline (SAH/SAM) ratios have been proposed in the mechanism of NTD development (see, for example, Stover, supra; Scott, 2001. Evidence of folic acid and folate in the prevention of neural tube defects. BibI Nutr Dieta 55:192-195. van der Put et al., 2001. Folate, Homocysteine and Neural Tube Defects: An Overview. Exptl Biol Med 226: 243-270.1, 5, 6).

Non-Folate Utilizing Enzymes Involved in Homocysteine Metabolism

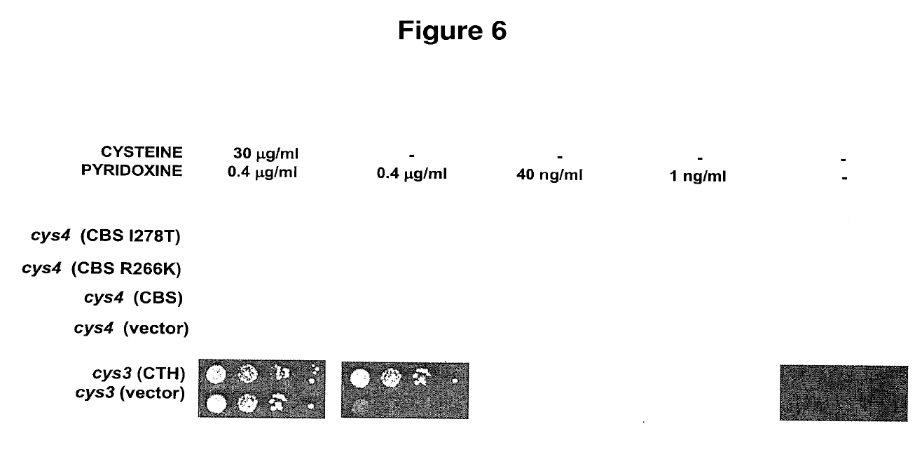

Cystathionine-f3-Synthase (CBS) defects result in elevated homocysteine levels and Cystathionine-3-Lyase (CTH) SNPs have been similarly associated with elevated homocysteine (see, for example, Kraus et al., supra; Wang et al., 2004. Single nucleotide polymorphism in CTH associated with variation in plasma homocysteine concentration. Clin Genet 65:483-486). Although not folate-utilizing enzymes, both CBS and CTH depend on a vitamin B₆-cofactor, and impaired alleles pose a risk of dysfunctional folate/homocysteine metabolism. Impaired alleles of CBS and CTH are targets for B₆ therapy, analogous to folate therapy for MTHFR impaired alleles as described herein. Function and vitamin-responsiveness of CBS and CTH are recapitulated in the yeast complementation assay. (FIG. 6).

Vitamin B-Remediation of CBS Mutant Enzymes is Recapitulated in S. cerevisiae

Yeast strains were engineered to assay CTH and CBS as a function of intracellular vitamin B₆ (pyridoxine) concentration (FIG. 6). The S. cerevisiae orthologs for CTH and CBS are cys3 and cys4, respectively, whose defect results in cysteine auxotrophy. Enzymes were tested as a function of pyridoxine concentration in a manner similar to that described herein for MTHFR except that the strain background is defective for pyridoxine biosynthesis (sextuple-delete sno1Δ sno2Δ sno3Δ snz1Δ snz2Δ snz3Δ; Stolz et al., 2003. Tpnlp, the plasma membrane vitamin B₆ transporter of Saccharomyces cerevisiae. J Biol Chem 278:18990-18996) as well as either a cys3 or cys4 defect.

FIG. 6 shows qualitative yeast growth assays on solid media and demonstrates that both enzymes rescue the cognate yeast defect as a function of pyridoxine supplementation and that the vitamin-responsiveness of two homocystinuria alleles of CBS (1278T, R266K) is recapitulated in this complementation assay: these alleles become more sensitive than the wild-type enzyme to limiting B₆ levels and show correspondingly greater growth defects. The rescue of cysteine auxotrophy in the cys4 mutant by human CBS has been demonstrated previously (Kruger et al. 1995. A yeast assay for functional detection of mutations in the human cystathionine—synthase gene. Hum Mol Genet 4:1155-1161; Kruger et al., 1994. A yeast system for expression of human cystathionine betasynthase: structural and functional conservation of the human and yeast genes. Proc NatI Acad Sci 91:6614-6618).

### Example 2

### Identification of Additional MTHFR Variants on a Sample Population

Genomic DNA was isolated from dried bloodspots (Guthrie Cards) of each of 250 newborns affected with a neural tube defect or each of 250 newborns not affected with a neural tube defect, The MTHFR exons in the isolated genomic DNA samples were sequenced as indicated in Example 1. Mutations that affect enzyme structure were identified from sequence data as mismatches against the consensus human genome sequence (NM_{—}005957). All substitutions are listed in Table A.

The functional impact of the MTHFR variants are tested using the in vivo yeast assay disclosed herein over a range of folate concentrations to observe functional effects as described in Example 1.

### Example 3

### Identification of ATIC, MTHFS, MAT1A, MAT2A and GART Variants

DNA Sample Population. Genomic DNA was isolated from dried bloodspots (Guthrie Cards) of each of 250 newborns affected with a neural tube defect or each of 250 newborns not affected with a neural tube defect. A total of 234 exons in 18 candidate genes from the folate/homocysteine metabolic pathway were sequenced. Sequencing and amplicon Mutations that affect enzyme structure were identified from sequence data as mismatches against the consensus human genome sequences listed in Table 2 for ATIC, MTHFS, MAT1A, MAT2A, and GART. All substitutions for ATIC, MTHFS, MAT1A, MAT2A, and GART are respectively listed in Tables B, C, D, E, and F.

The functional impact of the ATIC, MTHFS, MAT1A, MAT2A, and GART variants are tested over a range of folate concentrations using the disclosed in vivo yeast assay to observe functional effects as described in Example 1 and using the appropriate yeast strain backgrounds as described in Table 1.

All citations are expressly incorporated herein in their entirety by reference.

TABLE 4
Spectrum of nonsynonymous MTHFR alleles observed from sampling
over 500 unselected individuals of diverse ethnicity.

| | Length | Alleles | | |
|---|---|---|---|---|
| Exon | (bp) | Sequenced | Variant (codon) | Occurrences* |
| 1 | 236** | 1070 | None | |
| 2 | 239 | 1016 | M110I (atg−>atc) | 1 |
| | | | R134C (cgo--)tgc) | 1 |
| 3 | 111 | 1068 | None | |
| 4 | 194 | 1050 | A222V (gcc−>gtc) | 308 |
| | | | H213R (cac4cgc) | |
| | | | D223N (gat−>aat) | 1 |
| | | | | 1 |
| 5 | 251 | 1056 | D291N (gat-Mat) | 1 |
| 6 | 135 | 1042 | None | |
| 7 | 181 | 1062 | E429A (gaa->gca) | 251 |
| | | | G422R (ggg-agg) | 3 |
| 8 | 183 | 1058 | None | |
| 9 | 102 | 1072 | R519C (cgc−>tgc) | 2 |
| | | | R519L (cgc-)ctc) | 2 |
| 10 | 120 | 1072 | M581I (atg-Mta) | 1 |
| 11 | 219** | 1076 | R594Q (cgg4cag) | 47 |
| | | | T653M (acg−>atg) | 4 |
| | | | Q648P (cag-ccg) | 1 |

**for exons 1 and 11, only the length of the coding portion of the exon is given

TABLE 5
Recommended Vitamin Intake

| VITAMIN | CURRENT RDI * | | NEW DRI ** | | UL *** | |
|---|---|---|---|---|---|---|
| Vitamin A | 5000 | IU | 900 mcg (3000 IU) | | 3000 mcg (10,000 IU) | |
| Vitamin C | 60 | mg | 90 | mg | 2000 | mg |
| Vitamin D | 400 IU (10 mcg) | | 15 mcg (600 IU) | | 50 mcg (2000 IU) | |
| Vitamin E | 30 IU (20 mg) | | 15 | mg # | 1000 | mg |
| Vitamin K | 80 | mcg | 120 | mcg | ND | |
| Thiamin | 1.5 | mg | 1.2 | mg | ND | |
| Riboflavin | 1.7 | mg | 1.3 | mg | ND | |
| Niacin | 20 | mg | 16 | mg | 35 | mg |
| Vitamin B-6 | 2 | mg | 1.7 | mg | 100 | mg |

| Folate | 400 mcg (0.4 mg) | | 400 mcg from food, | | 1000 mcg synthetic |
|---|---|---|---|---|---|
| | | | 200 mcg synthetic ## | | |
| Vitamin B-12 | 6 | mcg | 2.4 | mcg ### | ND |
| Biotin | 300 | mcg | 30 | mcg | ND |
| Pantothenic acid | 10 | mg | 5 | mg | ND |

| | | | | | |
|---|---|---|---|---|---|
| Choline | Not established | 550 | mg | 3500 | mg |

* The Reference Daily Intake (RDI) is the value established by the Food and Drug Administration (FDA) for use in nutrition labeling. It was based initially on the highest 1968 Recommended Dietary Allowance (RDA) for each nutrient, to assure that needs were met for all age groups.
** The Dietary Reference Intakes (DRI) are the most recent set of dietary recommendations established by the Food and Nutrition Board of the Institute of Medicine, 1997-2001. They replace previous RDAs, and may be the basis for eventually updating the RDIs. The value shown here is the highest DRI for each nutrient.
*** The Upper Limit (UL) is the upper level of intake considered to be safe for use by adults, incorporating a safety factor. In some cases, lower ULs have been established for children.
# Historical vitamin E conversion factors were amended in the DRI report, so that 15 mg is defined as the equivalent of 22 IU of natural vitamin E or 33 IU of synthetic vitamin E.
## It is recommended that women of childbearing age obtain 400 mcg of synthetic folic acid from fortified breakfast cereals or dietary supplements, in addition to dietary folate.
### It is recommended that people over 50 meet the B-12 recommendation through fortified foods or supplements, to improve bioavailability.
ND Upper Limit not determined. No adverse effects observed from high intakes of the nutrient.
* obtained from the Council for Responsible Nutrition website

TABLE 6
Recommended Minearal Intake

| NUTRIENT | RDI* | | 1968 RDA** | | 1974 RDA** | | 1980 RDA** | | 1989 RDA** | | DRIs*** | |
|---|---|---|---|---|---|---|---|---|---|---|---|---|
| Calcium | 1000 | mg | 1300 | mg | 1200 | mg | 1200 | mg | 1200 | mg | 1300 | mg |
| Phosphorus | 1000 | mg | 1300 | mg | 1200 | mg | 1200 | mg | 1200 | mg | 1250 | mg |
| | | | | | | | | | | | (700 | adult) |
| Iron | 18 | mg | 18 | mg | 18 | mg | 18 | mg | 15 | mg | 18 | mg |
| Iodine | 150 | mcg | 150 | mcg | 150 | mcg | 150 | mcg | 150 | mcg | 150 | mcg |
| Magnesium | 400 | mg | 400 | mg | 400 | mg | 400 | mg | 400 | mg | 420 | mg |
| Zinc | 15 | mg | 10-15 | mg | 15 | mg | 15 | mg | 15 | mg | 11 | mg |

| Selenium | 70 | mcg | — | — | | | | 70 | mcg | 55 | mcg |
|---|---|---|---|---|---|---|---|---|---|---|---|
| Copper | 2 | mg | — | — | | 2-3 | mg | 1.5-3 | mg | 0.9 | mg |
| Manganese | 2 | mg | — | 2.5-7 | mg | 2.5-5 | mg | 2-5 | mg | 2.3 | mg |
| Chromium | 120 | mcg | — | — | | 50-200 | mcg | 50-200 | mcg | 35 | mcg |
| Molybdenum | 75 | mcg | — | 45-500 | mg | 150-500 | mcg | 75-250 | mcg | 45 | mcg |

*The Reference Daily Intake (RDI) is the value established by the Food and Drug Administration (FDA) for use in nutrition labeling. It was based initially on the highest 1968 Recommended Dietary Allowance (RDA) for each nutrient, to assure that needs were met for all age groups.
**The RDAs were established and periodically revised by the Food and Nutrition Board. Value shown is the highest RDA for each nutrient, in the year indicated for each revision.
***The Dietary Reference Intakes (DRI) are the most recent set of dietary recommendations established by the Food and Nutrition Board of the Institute of Medicine, 1997-2001. They replace previous RDAs, and may be the basis for eventually updating the RDIs. The value shown here is the highest DRI for each nutrient.
*obtained from the Council for Responsible Nutrition website

What is claimed is:

1. A formulation comprising a cofactor, wherein said cofactor is present in an amount determined by the genetic makeup of an individual.

2. The formulation of claim 1, comprising a plurality of cofactors, wherein at least a subset of said cofactors within said plurality is present in an amount determined by the genetic makeup of an individual.

3. The formulation of claim 1, wherein said cofactor is selected from the group consisting of: Vitamin A (retinol), Vitamin C (ascorbic acid), Vitamin D (calciferol), Vitamin E, Vitamin K (phylloquinone), Vitamin B1 (Thiamin), Vitamin B2 (riboflavin), Vitamin B3 (niacin), Vitamin B6 (pyridoxine), Vitamin B9 (folate/folic acid), Vitamin B12 (tocopherol), Vitamin B7 (biotin), Vitamin B5 (panthothenic acid), and choline.

4. The formulation of claim 2, wherein said plurality of cofactors comprises at least 2 cofactors selected from the group consisting of Vitamin A (retinol), Vitamin C (ascorbic acid), Vitamin D (calciferol), Vitamin E, Vitamin K (phylloquinone), Vitamin B1 (Thiamin), Vitamin B2 (riboflavin), Vitamin B3 (niacin), Vitamin B6 (pyridoxine), Vitamin B9 (folate/folic acid), Vitamin B12 (tocopherol), Vitamin B7 (biotin), Vitamin B5 (panthothenic acid), and choline.

5. The formulation of claim 1, wherein said formulation is prepared as a sustained release form.

6. The formulation of claim 1, wherein said formulation is orally ingestible.

7. The formulation of claim 1, wherein said formulation is prepared for intravenous, subcutaneous, or intramuscular administration.

8. The formulation of claim 1, wherein said formulation is prepared as a unit dosage.

9. The formulation of claim 1, wherein said formulation is prepared as a tablet or a capsule.

10. The formulation of claim 1, wherein said formulation is in liquid form.

11. The formulation of claim 1, wherein said genetic makeup comprises a genetic variant in one or more genes encoding one or more enzymes in a metabolic pathway, wherein said genetic variant is correlated to a cofactor remediable condition.

12. The formulation of claim 11, wherein said cofactor remediable condition is having an offspring with a neural tube defect.

13. The formulation of claim 11, wherein said cofactor remediable condition is selected from having an offspring with spina bifida, cleft palate, or anencephaly, or having a preterm birth.

14. The formulation of claim 1 accompanied by instructions for use by said individual.

15. A method of preparing the formulation of claim 1, comprising:
(a) selecting said cofactor; and
(b) mixing said cofactor with an excipient in an ingestible or injectable form.

16. The method of claim 15, wherein said step of selecting comprises selecting a plurality of cofactors, wherein at least a subset of said cofactors within said plurality is present in an amount determined by the genetic makeup of said individual.

17. The method of claim 15, wherein said cofactor is selected based on at least one personal characteristic of said individual, wherein said personal characteristic is selected from the group consisting of: weight, height, body-mass index, ethnicity, ancestry, gender, age, family history, medical history, exercise habit, and dietary habit.

18. A method of determining an amount of cofactor for an individual comprising:
(a) detecting the presence or absence of at least one genetic variant from a biological sample of said individual, wherein said at least one genetic variant correlates to a recommended amount of a cofactor that differs by at least 1% of the mass of said cofactor as compared to an amount recommended to an individual lacking said at least one genetic variant; and
(b) recommending said different amount of cofactor for said individual when said at least one genetic variant is detected in said biological sample.

19. The method of claim 18, wherein said genetic variant correlates to a recommended amount of a cofactor that differs by at least 1% greater than an amount recommended to an individual lacking said at least one genetic variant

20. The method of claim 18, wherein said genetic variant correlates to a recommended amount of a cofactor that differs by at least 1% less than an amount recommended to an individual lacking said at least one genetic variant

21. The method of claim 18, said genetic variant correlates to a recommended amount of a cofactor that differs by at least 500%.

22. The method of claim 18, wherein said individual is a female with a risk or predisposition for a cofactor remediable condition.

23. The method of claim 22, wherein said cofactor remediable condition is having an offspring with a neural tube defect.

24. The method of claim 22, wherein said cofactor remediable condition is selected from the group consisting of: having an offspring with spina bifida, cleft palate, or anencephaly; or having a preterm birth.

25. The method of claim 22, wherein said female is pregnant and said cofactor remediable condition is having an offspring with spina bifida.

26. A method of determining a risk or predisposition to a cofactor remediable condition in an individual comprising:
(a) detecting the presence or absence of a plurality of genetic variants from a biological sample of said individual, wherein said plurality of genetic variants is selected from Tables A-X; and,
(b) determining said predisposition to said cofactor remediable condition when said plurality of genetic variants is detected in said biological sample.

27. The method of claim 26, wherein said plurality of genetic variants comprises at least 2 genetic variants.

28. The method of claim 26, wherein said plurality of genetic variants comprises at least 3 genetic variants.

29. The method of claim 26, further comprising reporting said risk of a cofactor-dependent enzyme deficiency to said individual or a health care manager of said individual.

30. The method of claim 26, wherein said cofactor remediable condition is having an offspring with a neural tube defect.

31. The method of claim 26, wherein said cofactor remediable condition is selected from the group consisting of: having an offspring with spina bifida, cleft palate, or anencephaly; and having a preterm birth.

32. An isolated nucleic acid or a complement thereof, wherein said nucleic acid comprises a single nucleotide polymorphism (SNP) shown in Table A-X.

33. An array comprising immobilized thereon a plurality of isolated nucleic acids of claim of claim 32.

34. A computer assisted method of providing a personalized nutritional advice plan for an individual comprising:
(i) providing a first dataset on a data processing device, said first dataset comprising information correlating the presence of genetic variant of said individual, wherein the genetic variant indicates that the individual is at risk of a cofactor-dependent enzyme deficiency; and
(ii) providing a second dataset on a data processing device, said second dataset comprising information matching said co-factor-dependent enzyme deficiency with at least one lifestyle recommendation; and
(iii) generating a personalized nutritional advice plan based on the genetic variant of (i), wherein the plan comprises at least one lifestyle recommendation matched in step (ii).

35. The method of claim 34, wherein said personalized lifestyle advice plan includes recommended minimum and/or maximum amounts of vitamin subtypes.

36. The method of claim 34, wherein said personalized lifestyle advice plan includes recommended one or more cofactor in an amount based on the genetic variant of said individual.

37. The method of claim 34, wherein the method comprises the step of delivering the plan to the individual via Internet with the use of a unique identifier code.

38. The method of claim 34, wherein the method comprises the step of delivering the plan wirelessly to the individual or his/her agent.

39. The method of claim 34, wherein the method comprises the step of delivering the plan to the individual via an I-Phone®.

40. The method of claim 34, wherein the genetic variant of (ii) comprises a plurality of genetic variants correlated with one or more cofactor-dependent enzyme deficiencies.

41. The method of claim 40, wherein the one or more cofactor-dependent enzyme deficiencies is folate/folic acid deficiency.

42. The method of claim 34 further comprising a third dataset on a data processing device, said third dataset comprising information on one or more personal characteristics of said individual.

43. The method of claim 42, wherein said personal characteristic is selected from the group consisting of: weight, height, body-mass index, ethnicity, ancestry, gender, age, family history, medical history, exercise habit, and dietary habit.

44. The method of claim 34, wherein providing the first dataset of (i) and/or providing the second dataset of (ii) is carried out by inputting information of respective dataset by said individual or his/her agent.

45. The method of claim 34, wherein the plan comprises hyperlinks to one or more Web pages.

46. The method of claim 34, wherein the first data set comprises a plurality of genetic variants selected from Tables A-X.

47. A computer system comprising
(i) a data processing device configured to process a first dataset and/or a second data set, said first dataset comprising information correlating the presence of genetic variant of an individual, wherein the genetic variant indicates that the individual is at risk of a cofactor-dependent enzyme deficiency, and said second dataset comprising information matching said co-factor-dependent enzyme deficiency with at least one lifestyle recommendation; and
(ii) an output device configured to generate a personalized nutritional advice plan based on the genetic variant of said individual, wherein the plan comprises at least one lifestyle recommendation matched in (i).

48. The computer system of claim 47, further comprising an input device configured for inputting information on first data set and/or second data set.

49. The computer system of claim 48, wherein the input device is configured to input information on one or more personal characteristics of said individual.

50. A business method of providing a personalized nutritional advice plan for an individual, comprising:
(a) collecting information concerning the presence or absence of at least one genetic variant from a biological sample of said individual, wherein said at least one genetic variant correlates to a recommended amount of a cofactor that differs by at least 1% of said cofactor as compared to an amount recommended to an individual lacking said at least one genetic variant; and
(b) recommending said different amount of cofactor for said individual when said at least one genetic variant is detected in said biological sample.

51. The method of claim 50, wherein said genetic variant correlates to a recommended amount of a cofactor that differs by at least 1% greater than an amount recommended to an individual lacking said at least one genetic variant.

52. The method of claim 50, wherein said genetic variant correlates to a recommended amount of a cofactor that differs by at least 1% less than an amount recommended to an individual lacking said at least one genetic variant.

53. The method of claim 50, said genetic variant correlates to a recommended amount of a cofactor that differs by at least 500%.

54. The method of claim 50, wherein said individual is a female with a risk or predisposition for a cofactor remediable condition.

55. The method of claim 54, wherein said cofactor remediable condition is having an offspring with a neural tube defect.

56. The method of claim 54, wherein said cofactor remediable condition is selected from having an offspring with spina bifida, cleft palate, or anencephaly, or having a preterm birth.

57. The method of claim 54, wherein said individual is a pregnant female and said cofactor remediable condition is having an offspring with spina bifida.
